(19) United States
(12) Patent Application Publication
(10) Pub. No.: US 2025/0099478 A1
(43) Pub. Date: Mar. 27, 2025

(54) PLASMA KALLIKREIN INHIBITORS

(71) Applicant: Merck Sharp & Dohme LLC, Rahway, NJ (US)

(72) Inventors: Anthony K. Ogawa, San Mateo, CA (US); Christopher J. Sinz, Walnut Creek, CA (US); Alan C. Cheng, South San Francisco, CA (US); Ying-Duo Gao, Middletown, NJ (US); Song Yang, Springfield, NJ (US); Jianming Bao, Princeton, NJ (US); Rohan Rajiv Merchant, Burlingame, CA (US); Natalija Cernaka, Redwood City, CA (US); Phillip Patrick Sharp, San Francisco, CA (US); Jovan Alexander Lopez, New Haven, CT (US); Dong Xiao, Warren, NJ (US); Haiqun Tang, Belle Mead, NJ (US); Maoqun Tian, Foster City, CA (US); Mihir B. Mandal, Westfield, NJ (US); Jiafang He, Dayton, NJ (US)

(73) Assignee: Merck Sharp & Dohme LLC, Rahway, NJ (US)

(21) Appl. No.: 18/728,990

(22) PCT Filed: Jan. 23, 2023
(86) PCT No.: PCT/US2023/011304

§ 371 Date: Jul. 15, 2024

Related U.S. Application Data
(60) Provisional application No. 63/302,715, filed on Jan. 25, 2022.

(51) Int. Cl. A61K 31/537; A61K 45/06; C07D 498/10

(52) U.S. Cl. CPC A61K 31/537; A61K 45/06; C07D 498/10

(57) ABSTRACT

The present invention provides a compound of Formula (I) and pharmaceutical compositions comprising one or more said compounds, and methods for using said compounds for treating or preventing one or more disease states that could benefit from inhibition of plasma kallikrein, including hereditary angioedema, uveitis, posterior uveitis, wet age-related macular degeneration, diabetic macular edema, diabetic retinopathy and retinal vein occlusion. The compounds are selective inhibitors of plasma kallikrein.

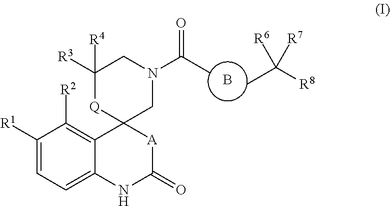

### BACKGROUND OF THE INVENTION

Plasma kallikrein is a zymogen of a trypsin-like serine protease and is present in plasma. The gene structure is similar to that of factor XI. Overall, the amino acid sequence of plasma kallikrein has 58% homology to factor XI. Proteolytic activation by factor XIIa at an internal I389-R390 bond yields a heavy chain (371 amino acids) and a light chain (248 amino acids). The active site of plasma kallikrein is contained in the light chain. The light chain of plasma kallikrein reacts with protease inhibitors, including alpha 2 macroglobulin and Cl-inhibitor. Interestingly, heparin significantly accelerates the inhibition of plasma kallikrein by antithrombin III in the presence of high molecular weight kininogen (HMWK). In blood, the majority of plasma kallikrein circulates in complex with HMWK. Plasma kallikrein cleaves HMWK to liberate bradykinin. Bradykinin release results in increase of vascular permeability and vasodilation (for review, Coleman, R., “Contact Activation Pathway”, Hemostasis and Thrombosis, pp. 103-122, Lippincott Williams & Wilkins (2001); Schmaier A. H., “Contact Activation”, Thrombosis and Hemorrhage, pp. 105-128 (1998)).

Patients presenting genetic deficiency on Cl-inhibitor suffer from hereditary angioedema (HAE), a lifelong disease that results in intermittent swelling throughout the body, including the hands, feet, face, throat, genitals and gastrointestinal tract. Analysis of blisters arising from acute episodes have been shown to contain high levels of plasma kallikrein, and treatment with a protein-based reversible plasma kallikrein inhibitor, Ecallantide (Kalbitor), has been approved by the FDA for the treatment of acute attacks of HAE (Schneider, L, et al., J. Allergy Clin. Immunol., 120: p. 416 (2007)). Recently, an oral plasma kallikrein inhibitor, Berotralstat, gained FDA approval for the prevention of HAE attacks (Zuraw, B., et al., J. Allergy Clin. Immunol. (2020).

Additionally, the plasma kallikrein-kinin system is abnormally abundant in patients diagnosed with advanced diabetic macular edema (DME). Recent publications have shown that plasma kallikrein contributes to observed retinal vascular leakage and dysfunction in diabetic rodent models (A. Clermont, et al., Diabetes, 60:1590 (2011)), and that treatment with a small molecule plasma kallikrein inhibitor ameliorated the observed retinal vascular permeability and other abnormalities related to retinal blood flow.

It would be desirable in the art to develop plasma kallikrein inhibitors having utility to treat a wide range of disorders, including hereditary angioedema, diabetic macular edema and diabetic retinopathy.

### SUMMARY OF THE INVENTION

The present invention relates to compounds of Formula I:

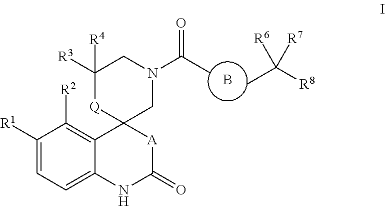

and pharmaceutically acceptable salts thereof. The compounds of Formula I are inhibitors of plasma kallikrein, and as such may be useful in the treatment, inhibition or amelioration of one or more disease states that could benefit from inhibition of plasma kallikrein, including hereditary angioedema, uveitis, posterior uveitis, wet age-related macular degeneration, diabetic macular edema, diabetic retinopathy and retinal vein occlusion. The compounds of this invention could further be used in combination with other therapeutically effective agents, including but not limited to, other drugs useful for the treatment of hereditary angioedema, uveitis, posterior uveitis, wet age-related macular degeneration, diabetic macular edema, diabetic retinopathy and retinal vein occlusion. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

The present invention relates to compounds of Formula I:

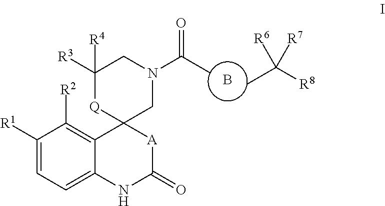

wherein A is O or —CH₂—;
[symbol] is

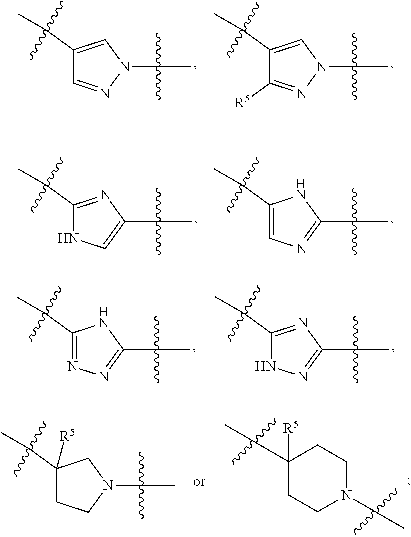

Q is —CH₂— or absent;
R¹ is selected from the group consisting of hydrogen, halo, hydroxy and C₁₋₆ alkyl;
R² is selected from the group consisting of hydrogen, halo, hydroxy and C₁₋₆ alkyl;
R³ is selected from the group consisting of hydrogen, halo, hydroxy, C₁₋₆ alkyl and C₃₋₆ cycloalkyl;
R⁴ is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3- to 6-membered cycloalkyl group, or a 5- to 6-membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

In an embodiment of the invention, Q is —CH₂—. In another embodiment of the invention, Q is absent.

In an embodiment of the invention, the present invention relates to compounds of the Formula Ia:

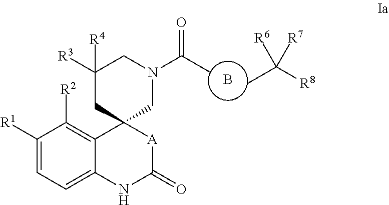

wherein A is O or —CH₂—;
[symbol] is

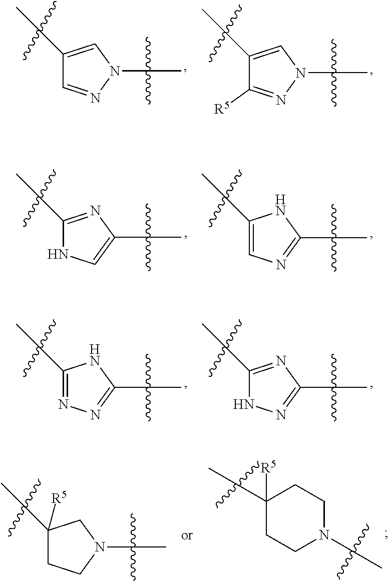

R¹ is selected from the group consisting of hydrogen, halo, hydroxy and C₁₋₆ alkyl;
R² is selected from the group consisting of hydrogen, halo, hydroxy and C₁₋₆ alkyl;
R³ is selected from the group consisting of hydrogen, halo, hydroxy, C₁₋₆ alkyl and C₃₋₆ cycloalkyl;
R⁴ is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3- to 6-membered cycloalkyl group, or a 5- to 6-membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

In an embodiment of the invention, A is O. In another embodiment of the invention, A is —CH₂—.

In an embodiment of the invention, the present invention relates to compounds of the Formula 1b:

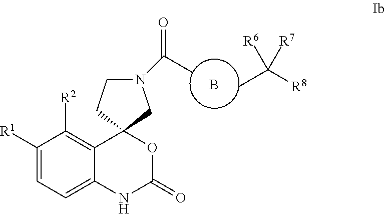

wherein [symbol] is

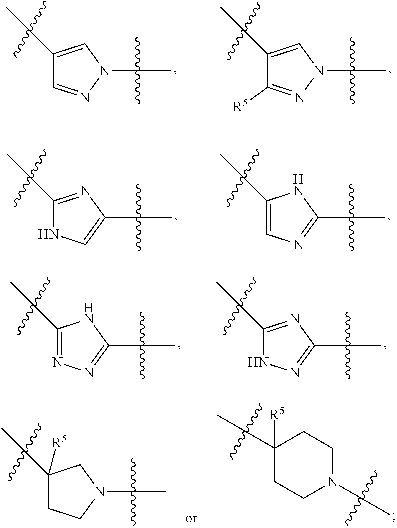

R¹ is selected from the group consisting of hydrogen, halo, hydroxy and C₁₋₆ alkyl;
R² is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3- to 6-membered cycloalkyl group, or a 5- to 6-membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

In an embodiment of the invention, [symbol] is

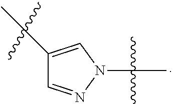 [SMILES: *c1cnn(*)c1]

In another embodiment of the invention, [symbol] is

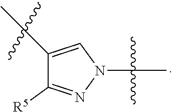

In another embodiment of the invention, [symbol] is

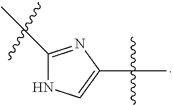 [SMILES: *c1c[nH]c(*)n1]

In another embodiment of the invention, [symbol] is

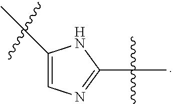 [SMILES: *c1cnc(*)[nH]1]

In another embodiment of the invention, [symbol] is

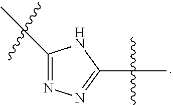 [SMILES: *c1nnc(*)[nH]1]

In another embodiment of the invention, [symbol] is

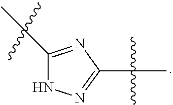 [SMILES: *c1n[nH]c(*)n1]

In another embodiment of the invention, [symbol] is

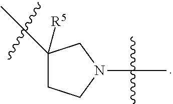

In another embodiment of the invention, [symbol] is

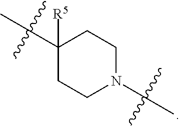

In an embodiment of the invention, R¹ is halo. In a class of the embodiment, R¹ is chloro.

In an embodiment of the invention, R² is halo. In a class of the embodiment invention, R² is fluoro.

In an embodiment of the invention, R³ is hydrogen or methyl. In a class of the embodiment, R³ is hydrogen. In a class of the embodiment, R³ is methyl.

In an embodiment of the invention, R⁴ is hydrogen or methyl. In a class of the embodiment, R⁴ is hydrogen. In a class of the embodiment, R⁴ is methyl.

In an embodiment of the invention, R⁵ is hydrogen or halo. In a class of the embodiment, R⁵ is halo. In a subclass of the embodiment, R⁵ is fluoro.

In an embodiment of the invention, R⁶ is hydrogen, hydroxyl, C₁₋₆-alkyl, and (C₁₋₆-alkyl)cyclopropyl, wherein said alkyl is optionally substituted with halo or OR^{x}. In a class of the embodiment, R⁶ is hydrogen, hydroxyl, C₁₋₆-alkyl, and —CH₂ (cyclopropyl), wherein said alkyl is optionally substituted with halo or OR^{x}. In a subclass of the embodiment, R⁶ is C₁₋₆-alkyl. In a further subclass of the embodiment, R⁶ is methyl. In another further subclass of the embodiment, R⁶ is ethyl. In another subclass of the embodiment, R⁶ is C₁₋₆-alkyl, which is substituted with OR^{x} or halo. In a further subclass of the embodiment, R⁶ is C₁₋₆-alkyl, which is substituted with fluoro, hydroxy or methoxy.

In an embodiment of the invention, R⁷ is hydrogen, hydroxyl or C₁₋₆-alkyl, which is optionally substituted with hydroxy or halo. In a class of the embodiment, R⁷ is hydrogen. In another class of the embodiment, R⁷ is hydroxyl. In another class of the embodiment, R⁷ is C₁₋₆-alkyl, which is optionally substituted with hydroxy or halo.

In an embodiment of the invention, R⁶ and R⁷ are taken together with the carbon atom to which they are attached to form a six-membered heterocycle or a C₃₋₆cycloalkyl group.

In an embodiment of the invention, R⁸ is phenyl, which is optionally substituted with oxo, halo, cyano, —OCH₂(C═O)OR^{x}, —SO₂R^{x}, and R^{y}. In a subclass of the embodiment, R⁸ is phenyl, which is substituted with fluoro or chloro. In another embodiment of the invention, R⁸ is a monocyclic or bicyclic heteroaryl ring, which is optionally substituted with halo, C₁₋₆-alkyl, R⁷ and NR⁹R¹⁰.

Reference to the preferred classes and subclasses set forth above is meant to include all combinations of particular and preferred groups unless stated otherwise.

Specific embodiments of the present invention include, but are not limited to the compounds identified herein as Examples 1 to 138, or pharmaceutically acceptable salts thereof.

Also included within the scope of the present invention is a pharmaceutical composition which is comprised of a compound of Formula I or Ia as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

The invention includes compositions for treating diseases or condition in which plasma kallikrein activity is implicated. Accordingly the invention includes compositions for treating impaired visual activity, diabetic retinopathy, diabetic macular edema, retinal vein occlusion, hereditary angioedema, diabetes, pancreatitis, cerebral hemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, and bleeding from postoperative surgery in a mammal, comprising a compound of the invention in a pharmaceutically acceptable carrier. A class of the invention includes compositions for treating hereditary angioedema, uveitis, posterior uveitis, wet age-related macular degeneration, diabetic macular edema, diabetic retinopathy and retinal vein occlusion. These compositions may optionally include anti-inflammatory agents, anti-VEGF agents, immunosuppressive agents, anticoagulants, antiplatelet agents, and thrombolytic agents. The compositions can be added to blood, blood products, or mammalian organs in order to effect the desired inhibitions.

The invention also includes compositions for preventing or treating retinal vascular permeability associated with diabetic retinopathy and diabetic macular edema in a mammal, comprising a compound of the invention in a pharmaceutically acceptable carrier. These compositions may optionally include anti-inflammatory agents, anti-VEGF agents, immunosuppressive agents, anticoagulants, antiplatelet agents, and thrombolytic agents.

The invention also includes compositions for treating inflammatory conditions of the eye, which includes, but is not limited to, uveitis, posterior uveitis, macular edema, acute macular degeneration, wet age-related macular degeneration, retinal detachments, retinal vein occlusion, ocular tumors, fungal infections, viral infections, multifocal choroiditis, diabetic uveitis, diabetic macular edema, diabetic retinopathy, proliferative vitreoretinopathy, sympathetic opthalmia, Vogt Koyanagi-Harada syndrome, histoplasmosis and uveal diffusion. These compositions may optionally include anti-inflammatory agents, anti-VEGF agents, immunosuppressive agents, anticoagulants, antiplatelet agents, and thrombolytic agents.

The invention also includes compositions treating posterior eye disease, which includes, but is not limited to, uveitis, posterior uveitis, wet age-related macular degeneration, diabetic macular edema, diabetic retinopathy and retinal vein occlusion. These compositions may optionally include anti-inflammatory agents, anti-VEGF agents, immunosuppressive agents, anticoagulants, antiplatelet agents, and thrombolytic agents.

It will be understood that the invention is directed to the compounds of structural Formula I or Ia described herein, as well as the pharmaceutically acceptable salts of the compounds of structural Formula I or Ia and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also included are basic nitrogen-containing groups which may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

If the compounds of Formula I or Ia simultaneously contain acidic and basic groups in the molecule the invention also includes, in addition to the salt forms mentioned, inner salts or betaines (zwitterions).

The present invention encompasses all stereoisomeric forms of the compounds of Formula I or Ia. Unless a specific stereochemistry is indicated, the present invention is meant to comprehend all such isomeric forms of these compounds. Centers of asymmetry that are present in the compounds of Formula I or Ia can all independently of one another have (R) configuration or (S) configuration. When bonds to the chiral carbon are depicted as straight lines in the structural Formulas of the invention, it is understood that both the (R) and (S) configurations of the chiral carbon, and hence both each individual enantiomer and mixtures thereof, are embraced within the Formula. When a particular configuration is depicted, that entantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

Unless a specific enantiomer or diastereomer is indicated, the invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or Ia, or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (1_{H}) and deuterium (2_{H}). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable (e.g. R^{x}, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I or Ia are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO⁻ depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

Except where noted herein, the terms “alkyl” and “alkylene” are intended to include both branched- and straight-chain saturated aliphatic hydrocarbon groups having the specified number of carbon atoms. Commonly used abbreviations for alkyl groups are used throughout the specification, e.g. methyl, may be represented by conventional abbreviations including “Me” or CH₃ or a symbol that is an extended bond as the terminal group, e.g. ‘ [symbol] ’, ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₄ alkyl” (or “C₁-C₄ alkyl”) for example, means linear or branched chain alkyl groups, including all isomers, having the specified number of carbon atoms. For example, the structures

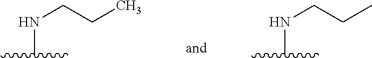

have equivalent meanings. C₁₋₄ alkyl includes n-, iso-, sec- and t-butyl, n- and isopropyl, ethyl and methyl. If no number is specified, 1-4 carbon atoms are intended for linear or branched alkyl groups.

Except where noted, the term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on.

Except where noted, the term “aryl”, as used herein, represents a stable monocyclic or bicyclic ring system of up to 10 carbon atoms in each ring, wherein at least one ring is aromatic. Bicyclic aryl ring systems include fused ring systems, where two rings share two atoms, and spiro ring systems, where two rings share one atom. Aryl groups within the scope of this definition include, but are not limited to: phenyl, indene, isoindene, naphthalene, and tetralin.

Except where noted, the term “heteroaryl”, as used herein, represents a stable monocyclic or bicyclic ring system of up to 10 atoms in each ring, wherein at least one ring is aromatic, and at least one ring contains from 1 to 4 heteroatoms selected from the group consisting of O, N and S. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzoxazolyl, carbazolyl, carbolinyl, cinnolinyl, dihydroindenyl, furanyl, indolinyl, indolyl, indolazinyl, indazolyl, isobenzofuranyl, isoindolyl, isoquinolyl, isothiazolyl, isoxazolyl, naphthalenyl, naphthpyridinyl, oxadiazolyl, oxazolyl, oxazoline, isoxazoline, pyranyl, pyrazinyl, pyrazolyl, pyrazolopyrimidinyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, dihydrobenzodioxinyl, dihydropyrazoloxazinyl, dihydropyrazolyothiazinedioxidyl, methylenedioxybenzene, benzothiazolyl, benzothienyl, quinolinyl, isoquinolinyl, oxazolyl, tetra-hydroquinoline, sulfolanyl, 1,3-benzodioxolyl, 3-oxo-3,4dihydro-2N-benzo[b][1,4]thiazine, imidazopyridinyl, 2-oxo-2,3-dihydroimidazolyl, 3,4-dihydrobenzoxazinyl, 2-oxo-2,3-dihydrooxazolyl, dihydroisobenzofuranyl, 1-oxoisoindolinyl, dioxido-2,3-dihydrobenzoisothiazolyl, and 2-oxopyridyl. If the heteroaryl contains nitrogen atoms, it is understood that the corresponding N-oxides thereof are also encompassed by this definition.

The term “heterocycle” or “heterocyclyl” as used herein is intended to mean a stable nonaromatic monocyclic or bicyclic ring system of up to 10 atoms in each ring, unless otherwise specified, containing from 1 to 4 heteroatoms selected from the group consisting of O, N, S, SO, or SO₂. Bicyclic heterocyclic ring systems include fused ring systems, where two rings share two atoms, and spiro ring systems, where two rings share one atom. “Heterocyclyl” therefore includes, but is not limited to the following: azaspirononanyl, azaspirooctanyl, azetidinyl, dioxanyl, isochromanyl, oxadiazaspirodecenyl, oxaspirooctanyl, oxazolidinonyl, piperazinyl, piperidinyl, pyrrolidinyl, morpholinyl, thiomorpholinyl, tetrahydrofumayl, tetrahydropyranyl, dihydropiperidinyl, tetrahydrothiophenyl and the like. If the heterocycle contains a nitrogen, it is understood that the corresponding N-oxides thereof are also encompassed by this definition.

Except where noted, the term “halogen” or “halo” means fluorine, chlorine, bromine or iodine.

“Celite®” (Fluka) diatomite is diatomaceous earth, and can be referred to as “celite”.

Except where noted herein, structures containing substituent variables such as variable “R” below:

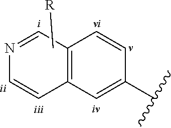

which are depicted as not being attached to any one particular bicyclic ring carbon atom, represent structures in which the variable can be optionally attached to any bicyclic ring carbon atom. For example, variable R shown in the above structure can be attached to any one of 6 bicyclic ring carbon atoms i, ii, iii, iv, v or vi.

Except where noted herein, bicyclic ring systems include fused ring systems, where two rings share two atoms, and spiro ring systems, where two rings share one atom.

The invention also relates to medicaments containing at least one compound of the Formula I or Ia and/or of a pharmaceutically acceptable salt of the compound of the Formula I or Ia and/or an optionally stereoisomeric form of the compound of the Formula I or Ia or a pharmaceutically acceptable salt of the stereoisomeric form of the compound of Formula I or Ia, together with a pharmaceutically suitable and pharmaceutically acceptable vehicle, additive and/or other active substances and auxiliaries.

The term “patient” used herein is taken to mean mammals such as primates, humans, sheep, horses, cattle, pigs, dogs, cats, rats, and mice.

The medicaments according to the invention can be administered by oral, inhalative, rectal or transdermal administration or by subcutaneous, intraarticular, intraperitoneal or intravenous injection. Oral administration is preferred. Coating of stents with compounds of the Formulas I and other surfaces which come into contact with blood in the body is possible.

The invention also relates to a process for the production of a medicament, which comprises bringing at least one compound of the Formula I or Ia into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the plasma kallikrein inhibitors is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the plasma kallikrein inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the plasma kallikrein inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/mL, e.g. 0.1 mg/mL, 0.3 mg/mL, and 0.6 mg/mL, and administered in amounts per day of between 0.01 mL/kg patient weight and 10.0 mL/kg patient weight, e.g. 0.1 mL/kg, 0.2 mL/kg, 0.5 mL/kg. In one example, an 80 kg patient, receiving 8 mL twice a day of an intravenous formulation having a concentration of active ingredient of 0.5 mg/mL, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

Compounds of Formula I or Ia can be administered both as a monotherapy and in combination with other therapeutic agents, including but not limited to anti-inflammatory agents, anti-VEGF agents, immunosuppressive agents, anticoagulants, antiplatelet agents, and thrombolytic agents.

An “anti-inflammatory agent” is any agent which is directly or indirectly effective in the reduction of inflammation when administered at a therapeutically effective level. “Anti-inflammatory agent” includes, but is not limited to steroidal anti-inflammatory agents and glucocorticoids. Suitable anti-inflammatory agents include, but are not limited to, cortisone, dexamethasone, hydrocortisone, methylprednisolone, prednisolone, prednisone and triamcinolone.

An “anti-VEGF agent” is any agent which is directly or indirectly effective in inhibiting the activity of VEGF (Vascular Endothelial Growth Factor). Suitable anti-VEGF agents include, but are not limited to, bevacizumab, ranibizumab, brolucizumab and aflibercept.

An “immunosuppressant agent” is any agent which is directly or indirectly effective in suppressing, or reducing, the strength of the body's immune system. Suitable immunosuppressant agents include, but are not limited to, corticosteroids (for example, prednisone, budesonide, prednisolone), janus kinase inhibitors (for example, tofacitinib), calcineurin inhibitors (for example, cyclosporin, tacrolimus), mTOR inhibitors (for example, sirolimus, everolimus), IMDH inhibitors (for example, azathioprine, leflunomide, mycophenolate), biologics (for example, abatacept, adalimumab, anakinra, certolizumab, etanercept, golimumab, infliximab, ixekizumab, natalizumab, rituximab, secukinumab, tocilizumab, ustekinumab, vedolizumab), and monoclonal antibodies (for example, basiliximab, daclizumab).

Suitable anticoagulants include, but are not limited to, factor XIa inhibitors, thrombin inhibitors, thrombin receptor antagonists, factor VIIa inhibitors, factor Xa inhibitors, factor IXa inhibitors, factor XIIa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis), other anticoagulants such as aspirin, and thrombolytic agents such as plasminogen activators or streptokinase to achieve synergistic effects in the treatment of various vascular pathologies. Such anticoagulants include, for example, apixaban, dabigatran, cangrelor, ticagrelor, vorapaxar, clopidogrel, edoxaban, mipomersen, prasugrel, rivaroxaban, and semuloparin. For example, patients suffering from coronary artery disease, and patients subjected to angioplasty procedures, would benefit from coadministration of fibrinogen receptor antagonists and thrombin inhibitors.

In certain embodiments the anti-inflammatory agents, anti-VEGF agents, immunosuppressant agents, anticoagulants, antiplatelet agents, and thrombolytic agents described herein are employed in their conventional dosage ranges and regimens as reported in the art, including, for example, the dosages described in editions of the Physicians' Desk Reference, such as the 70th edition (2016) and earlier editions. In other embodiments, the anti-inflammatory agents, anti-VEGF agents, immunosuppressant agents, anticoagulants, antiplatelet agents, and thrombolytic agents described herein are employed in lower than their conventional dosage ranges.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to anti-hypertensive agents, additional diuretics, anti-atherosclerotic agents such as a lipid modifying compound, anti-diabetic agents and/or anti-obesity agents may be used in any combination with the compound of the invention in a single dosage formulation (a fixed dose drug combination), or may be administered to the patient in one or more separate dosage formulations which allows for concurrent or sequential administration of the active agents (co-administration of the separate active agents). Examples of additional active agents which may be employed include but are not limited to angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); angiotensin II receptor antagonists also known as angiotensin receptor blockers or ARBs, which may be in free-base, free-acid, salt or pro-drug form, such as azilsartan, e.g., azilsartan medoxomil potassium (EDARBI®), candesartan, e.g., candesartan cilexetil (ATACAND®), eprosartan, e.g., eprosartan mesylate (TEVETAN®), irbesartan (AVAPRO®), losartan, e.g., losartan potassium (COZAAR®), olmesartan, e.g, olmesartan medoximil (BENICAR®), telmisartan (MICARDIS®), valsartan (DIOVAN®), and any of these drugs used in combination with a thiazide-like diuretic such as hydrochlorothiazide (e.g., HYZAAR®, DIOVAN HCT®, ATACAND HCT®), etc.); potassium sparing diuretics such as amiloride HCl, spironolactone, epleranone, triamterene, each with or without HCTZ; neutral endopeptidase inhibitors (e.g., thiorphan and phosphoramidon); aldosterone antagonists; aldosterone synthase inhibitors; renin inhibitors; enalkrein; RO 42-5892; A 65317; CP 80794; ES 1005; ES 8891; SQ 34017; aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazosin, prazosin or alpha methyldopa); central alpha adrenergic agonists; peripheral vasodilators (e.g. hydralazine); lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and particularly niacin in combination with a DP antagonist such as laropiprant and/or with an HMG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; inhibitors of glucose uptake, such as sodium-glucose transporter (SGLT) inhibitors and its various isoforms, such as SGLT-1, SGLT-2 (e.g., ASP-1941, TS-071, BI-10773, tofogliflozin, LX-4211, canagliflozin, dapagliflozin, ertugliflozin, ipragliflozin, remogliflozin and sotagliflozin), and SGLT-3; or with other drugs beneficial for the prevention or the treatment of the above-mentioned diseases including but not limited to diazoxide; and including the free-acid, free-base, and pharmaceutically acceptable salt forms, pro-drug forms, e.g., esters, and salts of pro-drugs of the above medicinal agents, where chemically possible. Trademark names of pharmaceutical drugs noted above are provided for exemplification of the marketed form of the active agent(s); such pharmaceutical drugs could be used in a separate dosage form for concurrent or sequential administration with a compound of the invention, or the active agent(s) therein could be used in a fixed dose drug combination including a compound of the invention.

Typical doses of the plasma kallikrein inhibitors of the invention in combination with other suitable agents may be the same as those doses of plasma kallikrein inhibitors administered without coadministration of additional agents, or may be substantially less that those doses of plasma kallikrein inhibitors administered without coadministration of additional agents, depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e., prevent, inhibit or ameliorate) the disease condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. The administration of each component does not need to be via the same route of administration; for example, one component can be administered orally, and another can be delivered into the vitreous of the eye.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

### General Methods

Compounds of the present invention may be prepared using conventional techniques or according to the methodology outlined in the following general synthetic schemes. One skilled in the art can vary the procedures and reagents shown to arrive at similar intermediates and/or final compounds.

NMR spectra were measured on VARIAN or Bruker NMR Systems (400, 500 or 600 MHz). Chemical shifts are reported in ppm downfield and up field from tetramethylsilane (TMS) and referenced to either internal TMS or solvent resonances (¹H NMR: δ 7.27 for CDCl₃, δ 2.50 for (CD₃)(CHD₂)SO, and ¹³C NMR: δ 77.02 for CDCl₃, δ 39.51 for (CD₃)₂SO. Coupling constants (J) are expressed in hertz (Hz), and spin multiplicities are given as s (singlet), d (doublet), dd (double doublet), t (triplet), m (multiplet), and br (broad). Chiral resolutions can be performed on either Waters Thar 80 SFC or Berger MG II preparative SFC systems. LC-MS data can be recorded on SHIMADAZU LC-MS-2020, SHIMADAZU LC-MS-2010, or Agilent 1100 series LC-MS, Agilent Prime-1260, or Waters Acquity LC-MS instruments using C18 columns employing a MeCN gradient in water containing 0.02 to 0.10% TFA. UV detections were at 220 and/or 254 nm and ESI ionization was used for MS detection.

When chiral resolution was achieved by chromatography using chiral columns, the chiral columns used for SFC chiral resolutions are listed in tables. Some of the chiral columns used were CHIRALPAK AD, CHIRALCEL OJ, CHIRALPAK AS, CHIRALPAK AY, CHIRALPAK IA, CHIRALPAK AD-H, and CHIRALPAK AS-H. Henceforth, they will be referred by their two or three letter abbreviations. As a convention, the fast-eluting isomer from a chiral resolution is always listed first in this table followed immediately by the slower-eluting isomer from the same resolution. If more than two isomers were separated, they will be always listed in the tables in order they were eluted, such as Peak 1 followed by Peak 2, Peak 3 and so on. A * symbol near a chiral center in a structure denotes that this chiral center was resolved by chiral resolution without its stereochemical configuration unambiguously determined.

Also, UV is ultraviolet; W is watts; wt. % is percentage by weight; ×g is times gravity; α_{D} is the specific rotation of polarized light at 589 nm; % w/v is percentage in weight of the former agent relative to the volume of the latter agent; % v/v is percentage in volume of the former agent relative to the volume of the latter agent; cpm is counts per minute; δ_{H} is chemical shift; and a mass spectrum obtained by ES-MS may be denoted herein by “LC-MS”; m/z is mass to charge ratio; n is normal; nm is nanometer; nM is nanomolar.

For purposes of this specification, the following abbreviations have the indicated meanings:
Ac acetyl
ACN acetonitrile
AcOH oracetic acid
HOAc
aq. aqueous
Ar aryl
Atm atmospheric pressure
Bn benzyl
Boc or BOC tert-butoxycarbonyl
Br broad
nBu butyl
° C. degrees Celsius
calcd. calculated
CDI 1,1′-Carbonyldiimidazole
D day
Δ chemical shift
D doublet
DAST (diethylamino)sulfur trifluoride
DCM dichloromethane
Dd doublet of doublets
DEAD diethylazodicarboxylate
DIAD diisopropylazodicarboxylate
DIEA, DIPEAN,N-diisopropylethylamine or Hünig's base
DIPA diisopropylamine
DMF dimethylformamide
DMI 1,3-dimethylimidazolodinone
DMP Dess-Martin periodinane (1,1,1-triacetoxy)-1,1-dihydro-1,2-benziodoxol-3(1H)-one
DMSO dimethyl sulfoxide
Dppf 1,1′-bis(diphenylphosphino)ferrocene
Dq doublet of quartets
Dt doublet of triplets
DTBDP 2,6-di-tert-butylpyridine
DTT dithiothreitol
EDC or EDCI 1-ethyl-3-(3-dimethylaminopropyl)carbodiimide
EDTA ethylenediamine tetraacetic acid
Equiv equivalents
ESI electrospray ionization
Et ethyl
EtOH ethanol
EtOAc ethyl acetate
g Grams
GST glutathione S-transferase
h Hour
HATU N,N,N′,N′-tetramethyl-O-(7-azabenzotriazol-1-yl)uronium hexafluorophosphate
HMDS 1,1,1,3,3,3-hexamethyldisilazane
HOBt 1-hvdroxybenzotriazole
HPLC high-performance liquid chromatography
Hz Hertz
IC₅₀ concentration at which 50% inhibition exists
IPA isopropanol
iPr isopropyl
J coupling constant
KHMDS potassium bis(trimethylsilyl)amide
L liters
LC liquid chromatography
LCMS liquid chromatography mass spectrometry
LDA lithium diisopropylamide
LED light emitting diode
LHMDS lithium bis(trimethylsilyl)amide
M mass
M molar
M multiplet
Me methyl
MeOH methanol
Mg milligrams
MHz megahertz
Min minute
μL Microliters
mL Milliliters
Mmol Millimoles
MPLC medium pressure liquid chromatography
MS mass spectrometry
Ms methanesulfonyl (mesyl)
N normal
NCS N-chlorosuccinimide
NMR nuclear magnetic resonance spectroscopy
P pentet
pH pH to indicate the acidity or basicity of an aqueous solution
Ph phenyl
PMB 4-methoxybenzyl
Pr propyl
Psi pounds per square inch
Q quartet
Qd quartet of doublets
Rac racemic mixture
RT or rt room temperature (ambient, about 25° C.)
s singlet
satd. saturated
SEM 2-(trimethylsilyl)ethoxymethyl
SFC supercritical fluid chromatography
S_{N}Ar nucleophilic aromatic substitution
t triplet
T₃P propylphosphonic anhydride
TBAD di-tert-butyl azodicarboxylate
TBAI tetrabutylammonium iodide
tBu tert-butyl
Td triplet of doublets
TEA triethylamine (Et₃N)
TFA trifluoroacetic acid
THF tetrahydrofuran
TLC thin layer chromatography
TMS trimethylsilyl
Tris tris(hydroxymethyl)aminomethane

### General

Starting materials used were obtained from commercial sources or prepared in other examples, unless otherwisely noted. The methods used for the preparation of the compounds of this invention are illustrated by the following schemes. Unless specified otherwise, all starting materials used are commercially available.

### Schemes

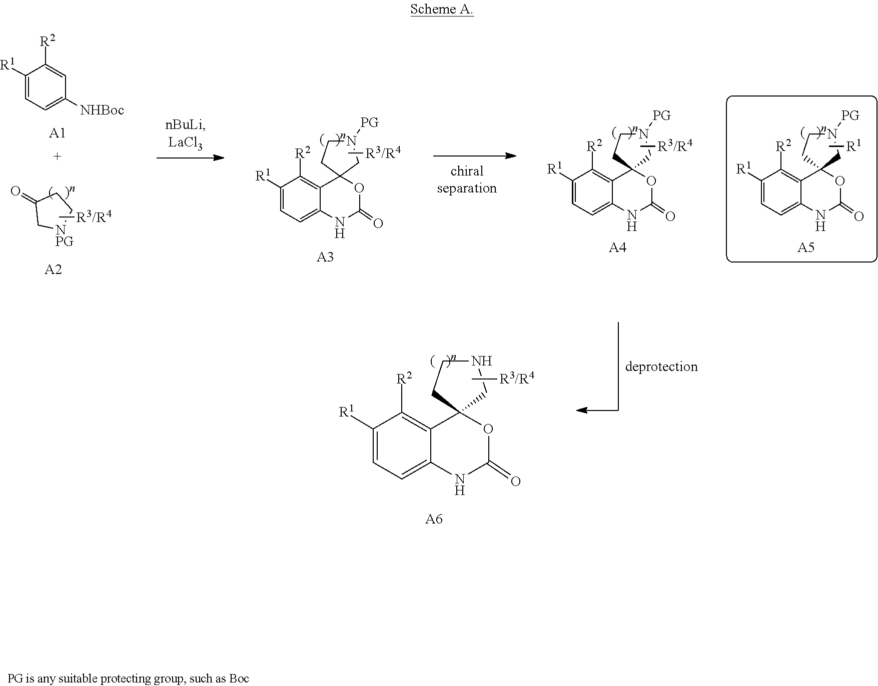

Scheme A illustrates the synthetic sequence for preparation of substituted spirocarbamates such as A6 from Boc-protected aniline A1 and ketones such as A2. Directed lithiation of aniline A1 and addition into the heterocyclic ketone A2 occurs in the presence of Lewis acid (eg. LaCl₃). The tertiary alcohol undergoes in situ cyclization onto the carbamate to give spirocarbamate derivatives such as A3, which can be subjected to chiral separation, preferably using supercritical flow chromatography (SFC) to afford enantiomers A4 and A5. Deprotection of either enantiomer (A4. e.g.) gives the secondary amine A6 that can be carried on to compounds of this invention.

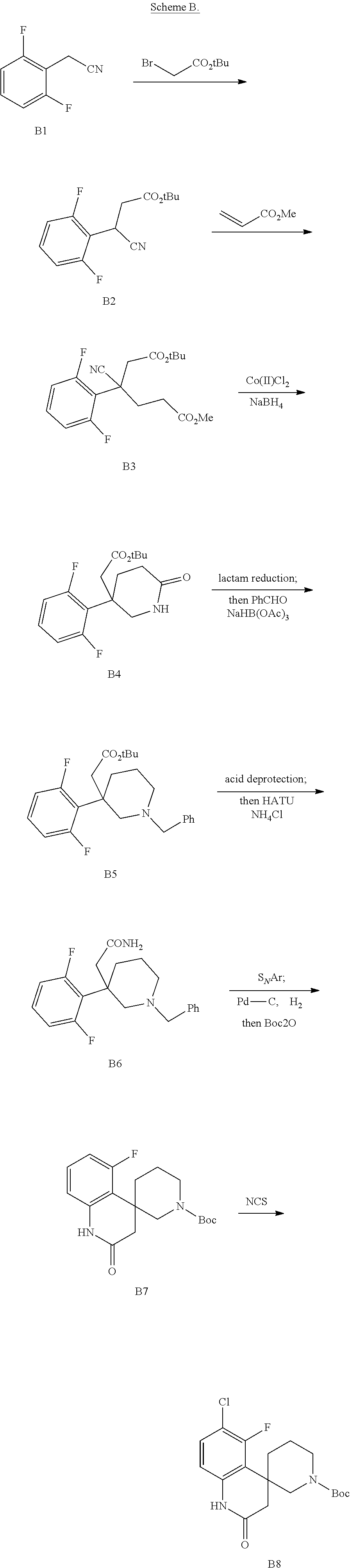

Scheme B illustrates a synthetic sequence for the preparation of spirolactams such as B8. Difluorophenylacetonitrile B1 is reacted with a bromoacetate input under basic conditions to afford the 3-cyano-3-arylpropionate B2 that is further reacted with methyl acrylate to afford mixed ester B3. Cobalt-catalyzed reduction of the nitrile, and in situ cyclization affords a lactam (B4) that is reduced to the piperidine and benzylated to afford ester B5. Ester hydrolysis, followed by formation of the primary amide (B6) produces a synthon that can be further reacted to effect SNAr displacement of an ortho-fluorine to yield the spirolactam skeleton (B7). This core structure can be further elaborated to afford the desired aryl ring functionality (B8), and the two enantiomers can be separated by chiral chromatography that can be carried on to compounds of this invention.

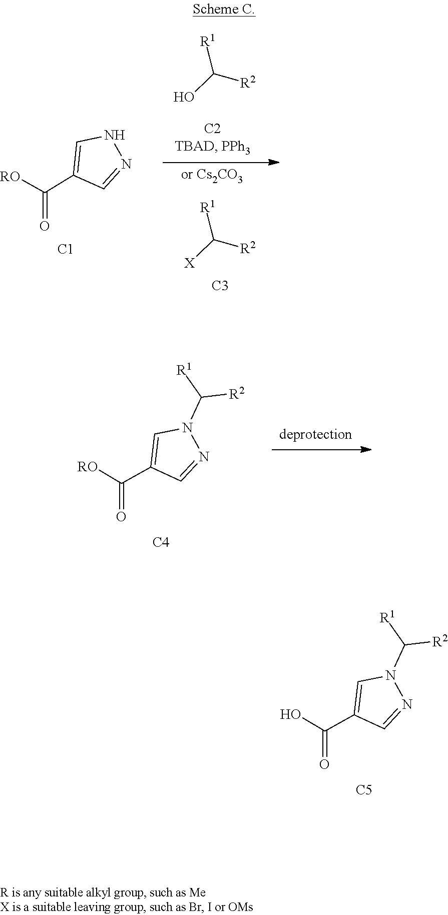

Scheme C illustrates a synthetic sequence for the preparation of N-alkylpyrazoles such as C5. Pyrazole C1 can either be subjected to Mitsunobu reaction with alcohol C2, or reacted with alkyl halides C3 in the presence of a suitable weak base, such as Cs₂CO₃, to afford N-alkylated esters of type C4 that can be deprotected under suitable reaction conditions to afford carboxylic acids (C5) that can be carried on to compounds of this invention.

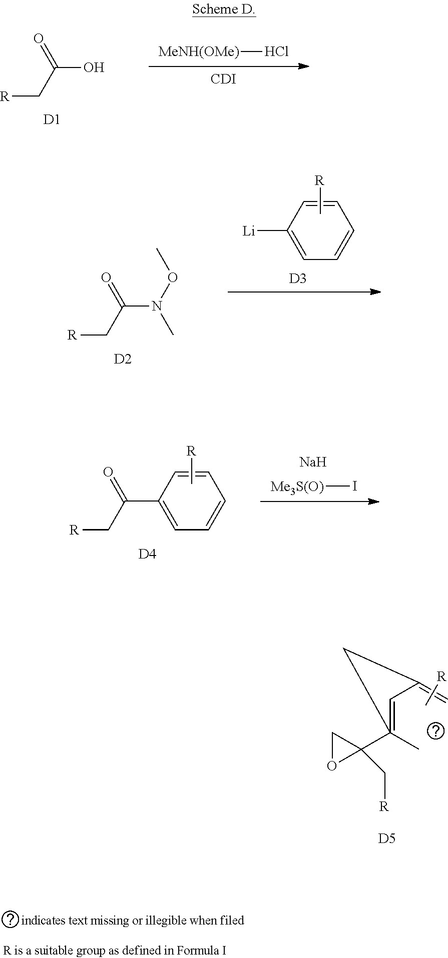

Scheme D illustrates a synthetic sequence for the preparation of epoxides such as D5 from carboxylic acids of type D1. Reaction of D1 with methoxymethylamine in the presence of a suitable coupling agent, such as CDI, can give a Weinreb-type amide D2. This intermediate can be reacted with aryllithium reagents (D3), generated by in situ lithium-halogen exchange by treating the corresponding aryl bromide and n-butyl lithium at −78° C., to give aryl ketones of type D4. Further reaction of D4 with the sulfoxonium ylides, derived from trimethylsulfoxonium iodide and a strong base, can yield an epoxide (D5) that can be carried on to compounds of this invention.

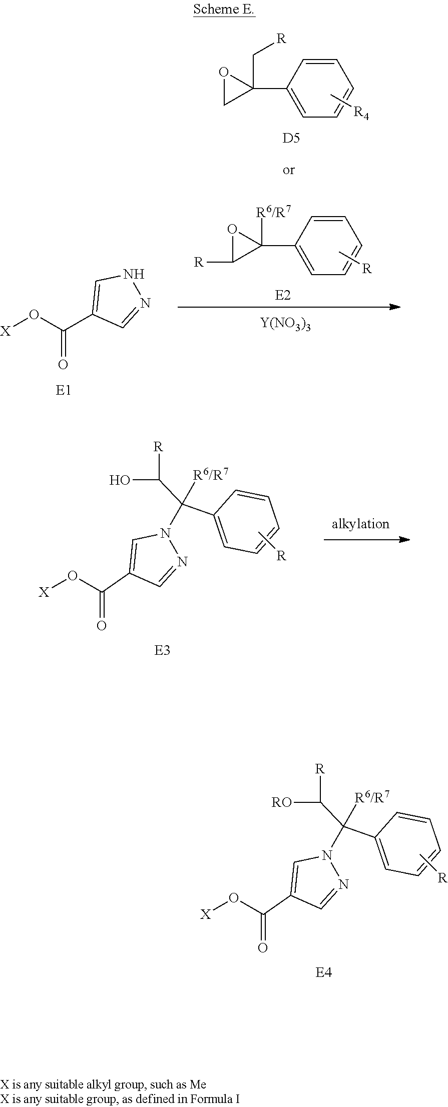

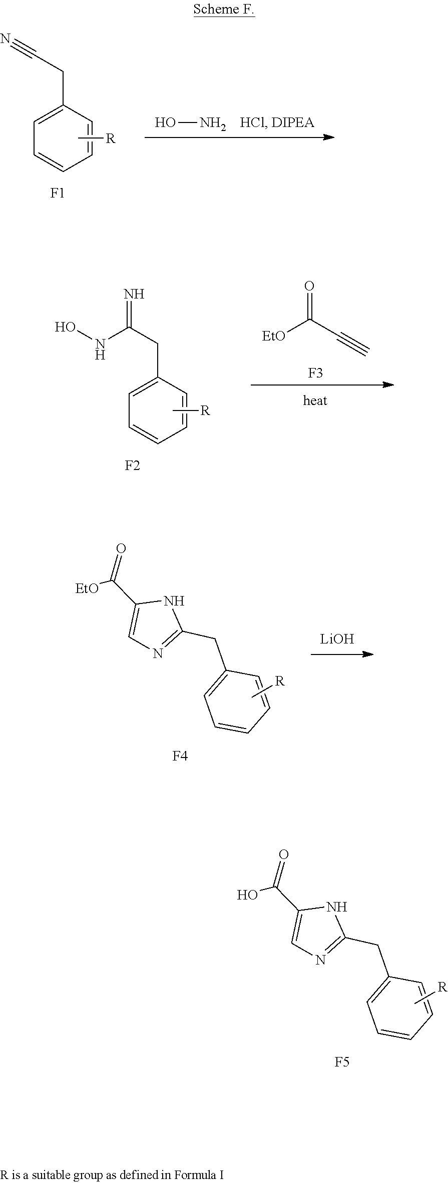

Scheme F illustrates a synthetic sequence for the preparation of imidazole esters F4 and carboxylic acids F5 from nitriles (F1). Nitriles such as F1 are treated with hydroxylamine hydrochloride to afford a hydroxy amidines of type F2. Heating the hydroxyamidine (F2) with an alkynoate (F3) affords the target imidazole esters (F4) that can be saponified to the corresponding carboxylic acid (F5), and subsequently, carried on to compounds of this invention.

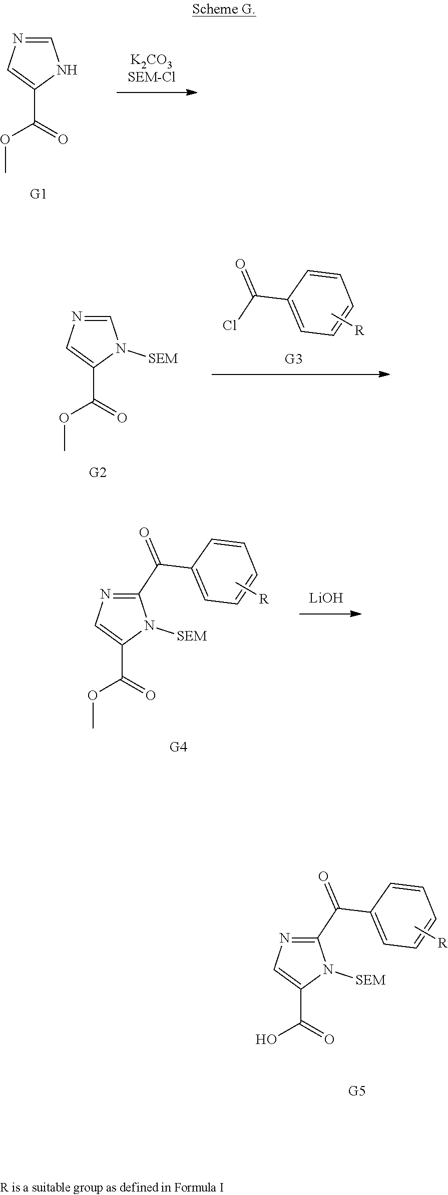

Scheme G illustrates a synthetic sequence for the preparation of imidazophenones G4 from imidazolocarboxylates G1. Imidazole G1 can be protected with SEM chloride, which facilitates regiospecific acylation with suitable benzoyl chloride G3 inputs to afford the target inidazolophenones G4, which can be saponified to afford a carboxylic acid intermediate (G5) that can be carried on to compounds of this invention.

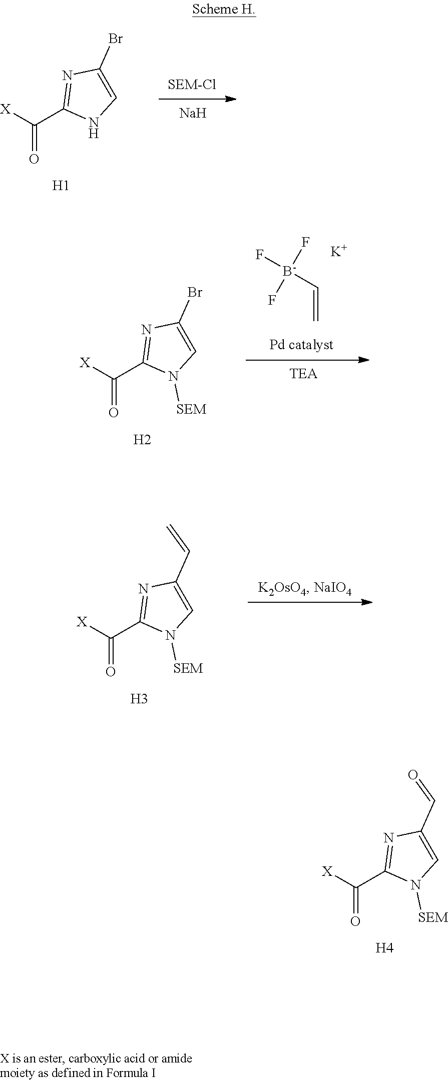

Scheme H illustrates a synthetic sequence for the preparation of imidazocarboxaldehyde H4. Bromoimidazole H1 can be protected by treatment with a suitable reagent, such as SEM-Cl, and coupled with vinyl potassium tetrafluoroborate salt, in the presence of a palladium catalyst, to yield vinylimidazole H3. Oxidative cleavage of the olefin under Lemieux-Johnson conditions using sodium periodate and potassium osmate affords the desired imidazocarboxaldehyde H4 that can be carried on to compounds of this invention.

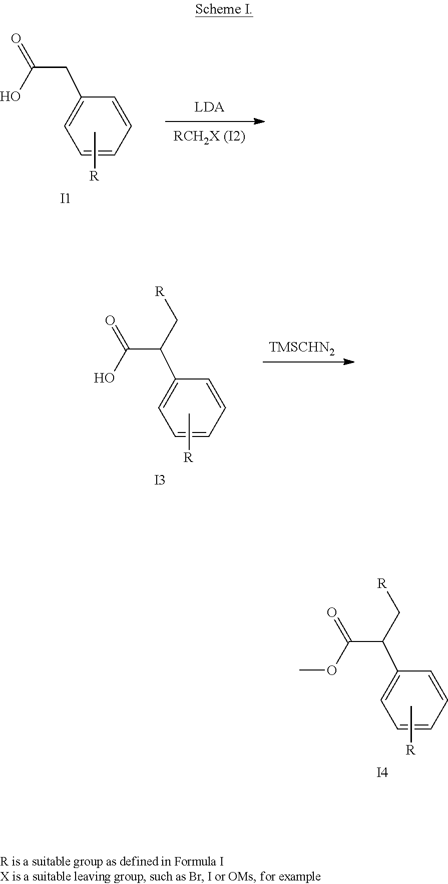

Scheme I illustrates a synthetic sequence for the preparation of substituted phenylacetates I4 from a phenylacetic acid (I1) starting material. Treatment of I1 with a suitable base, such as LDA, followed by addition of a suitable alkylating reagent (I2) can afford the desired mono-alkylated acid I3. Esterification by a number of methods known to those skilled in the art (reaction with TMS-CHN₂, e.g.) can yield the desired alkylated ester I4 that can be carried on to compounds of this invention.

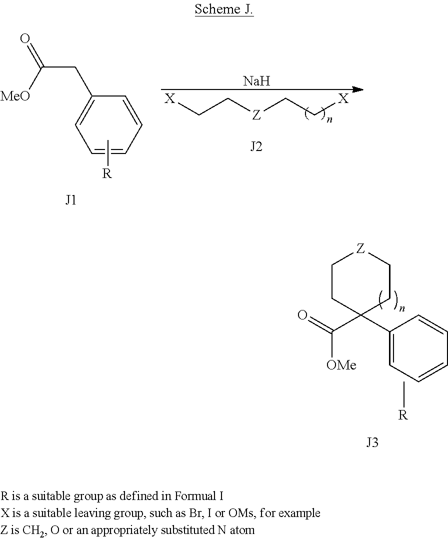

Scheme J illustrates a method for generating α-spirophenylacetates J3, where in this instance, an unsubstituted phenylacetate (J1) is reacted with a bifunctional alkylating agent (J2) in the presence of a suitable base, such as NaH, to afford the desired α,α-disubstituted phenylacetate (J3) that can be carried on to compounds of this invention.

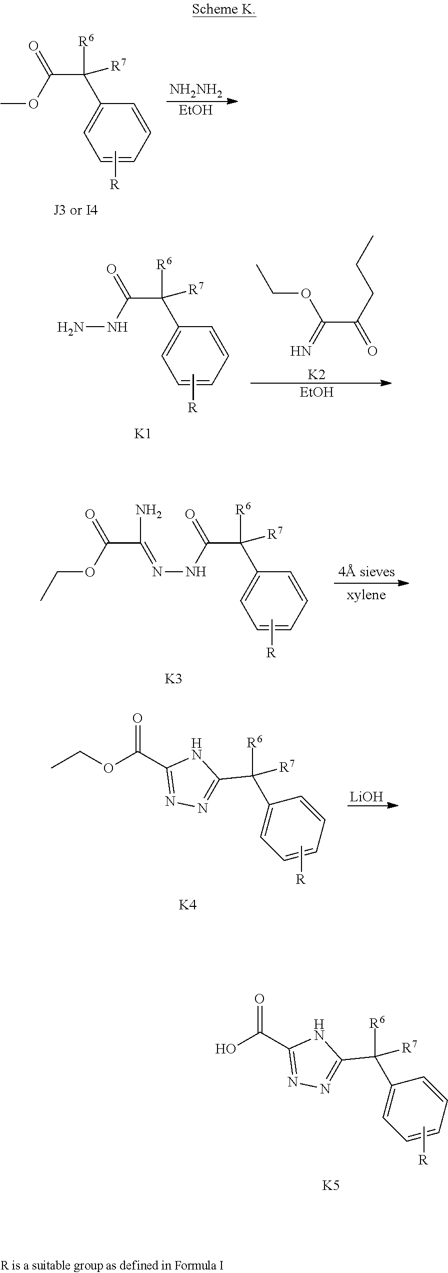

Scheme K illustrates a synthetic sequence for the preparation of 1,2,4-triazoles K3 from esters, such as I4 or J3. Reaction of I4 or J3 with hydrazine affords a hydrazide intermediate K1 that is subsequently condensed with an imidate (K2) to give an aminocarbazone K3. Thermal cyclization of K3 in the presence of a suitable drying agent, such as molecular sieves, can afford the desired 1,2,4-triazole ester K4 that can be carried on to compounds of this invention.

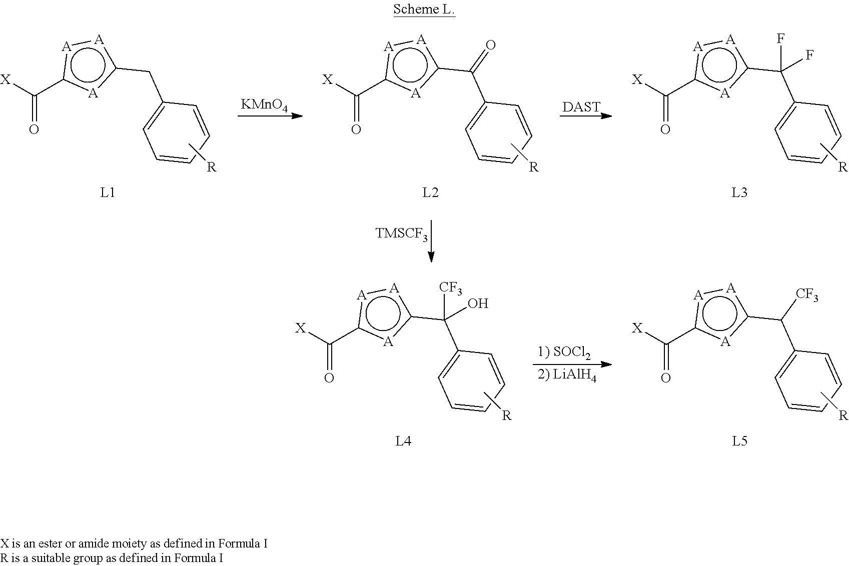

Scheme L illustrates a synthetic sequence to convert benzyl-substituted heterocycles (Li) into phenone intermediates L2 that can be functionalized further at the benzylic carbon. Benzylic oxidation of heterocycles of type L1 with an appropriate oxidant, commonly potassium permanganate, can directly give the aforementioned phenone intermediate L2. Reaction of L2 in the presence of a fluorinating agent, such as DAST, can yield α,α-gem-difluorobenzylic heterocycles L3. Alternatively, treatment of L2 with trimethylsilyltrifluoromethane, followed by a 2-step process involving conversion of the hydroxyl to a reactive halide L4 (conversion to a chloride with thionyl chloride, e.g.) followed by reduction with suitable agent, such lithium aluminum hydride, can afford an α-trifluormethyl-substituted benzylic heterocycle L5 that can be carried on to compounds of this invention. In addition, any intermediate examples of L2-L5, wherein X is defined to result in an ester functionality, can be treated as described above to yield the corresponding carboxylic acids (not shown) that can likewise be carried on to compounds of this invention.

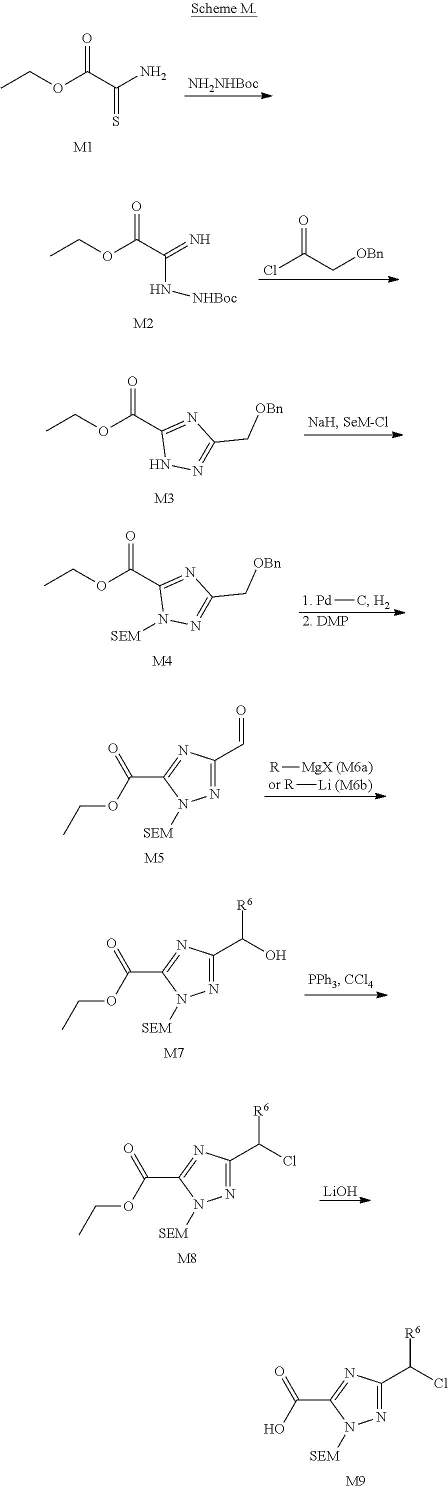

Scheme M illustrates a preferred method for generating desired intermediates such as triazolocarboxaldehyde (M5) from aminothioxoacetate M1. Reaction of M1 with Boc-hydrazine, followed by treatment with benzyloxyacetyl chloride and heating resulted in a 5-substituted-3-carboxytriazole (M3). SEM-protection (M4), followed by debenzylation and oxidation of the resultant hydroxyl moiety using methods known to those skilled in the art an afford a triazole carboxaldehyde M5. Treatment with an appropriate nucleophile, such as a Grignard reagent (M6a) or alkyllithium species (M6b), can give a 2°-alcohol M7 that can itself be carried on or reacted further in the presence of carbon tetrachloride and triphenylphosphine to afford a chloromethyl triazolecarboxylate M8 that can be carried on directly or saponified to the carboxylic acid M9, which can be carried on to compounds of this invention.

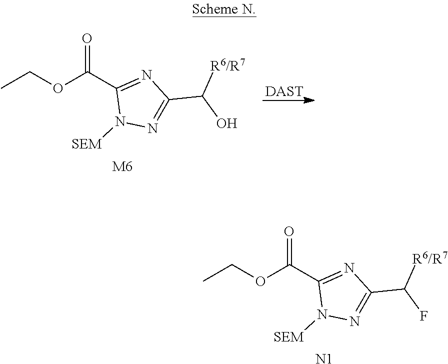

Scheme N illustrates a method for converting secondary alcohol M6 to the corresponding alkylfluoride (N1) by reacting M6 in the presence of DAST that can be carried on to compounds of this invention.

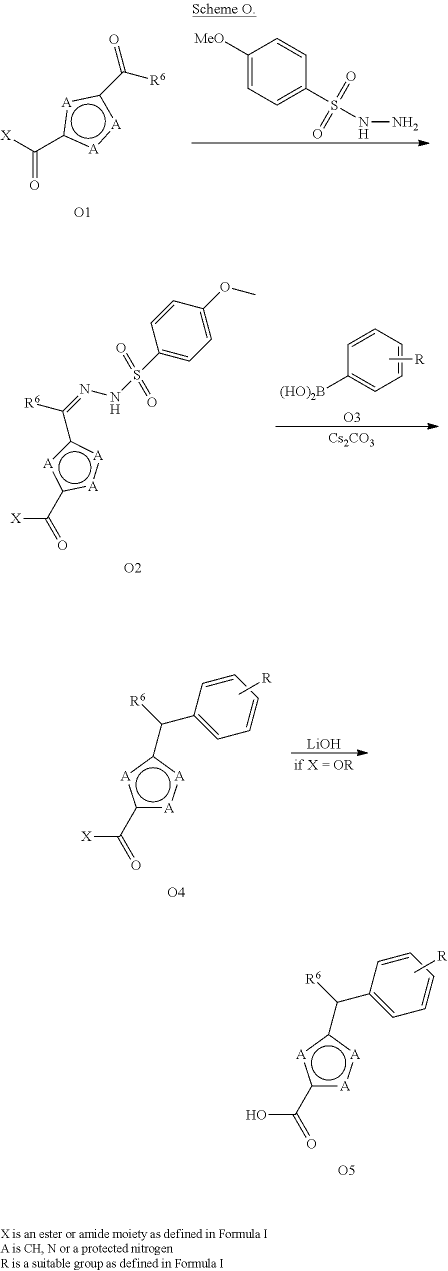

Scheme O illustrates a synthetic sequence for the conversion of heteroaryl ketones or aldehydes (O1) to benzyl-substituted heterocycles O4 and O5. Ketones or aldehydes, such as O1, can be condensed with arylsulfonylhydrzides to afford hydrazones O2, which can be subjected to Barluenga-type coupling with arylboronic acids (O3), in the presence of an appropriate base, such as Cs₂CO₃, to afford the desired benzyl-substituted heterocycles O4 that can be carried on directly or saponified to carboxylic acid O5 which can be carried on to compounds of this invention.

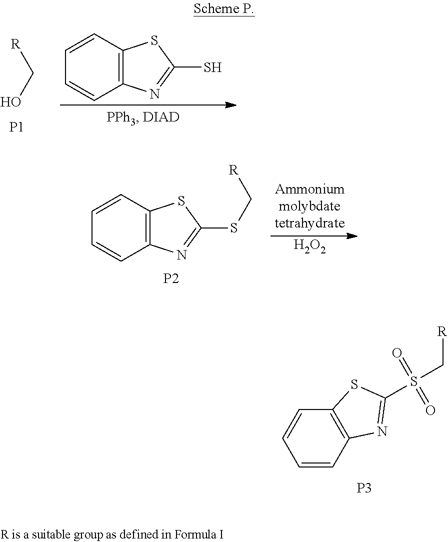

Scheme P illustrates a synthetic sequence for the preparation of sulfones such as P4 in a 2 step sequence from an alcohol starting material (P1). Alcohol P1 can be reacted with thiobenzothiazole under Mitsunobu conditions to give a thioether intermediate P2, which can subsequently be oxidized to afford substituted sulfones P3 that can be carried on to compounds of this invention.

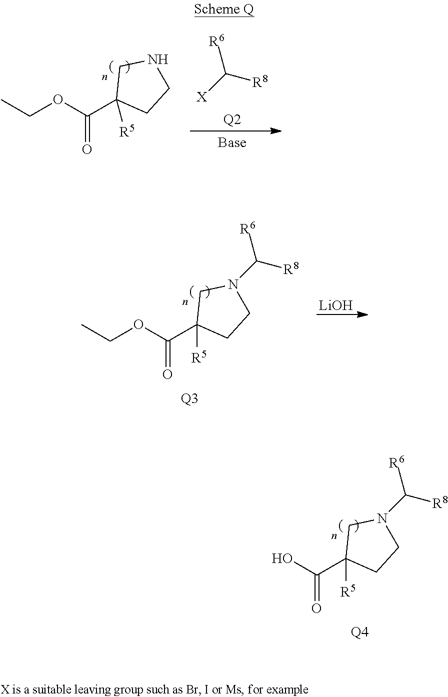

Scheme Q illustrates a synthetic sequence for the preparation of N-alkylpyrrolidines, and piperidines such as Q3. Heterocycle Q1 can be reacted with an appropriate alkyl halide Q2 in the presence of a suitable, non-nucleophilic base, such as NaH or Cs₂CO₃, to afford N-alkylated esters of type Q3 that can be carried on directly or saponified to the carboxylic acid Q4 which can be carried on to compounds of this invention.

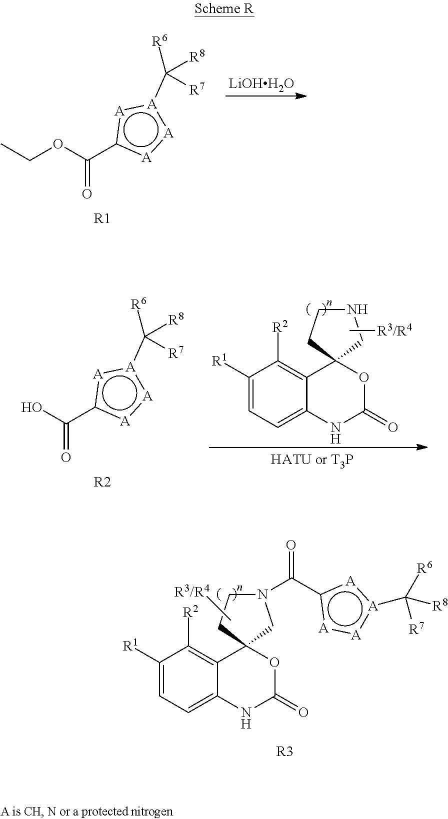

Scheme R illustrates a synthetic sequence for the preparation of spirocarbamate amides R³ employing the corresponding ester R¹ and spirocarbamate A6 synthons. Ester R¹ can be saponified to afford a carboxylic acid R², which is subsequently reacted with spirocarbamate A6 in the presence of a suitable coupling agent, such as HATU or T3P, to afford R³, which represents compounds of this invention.

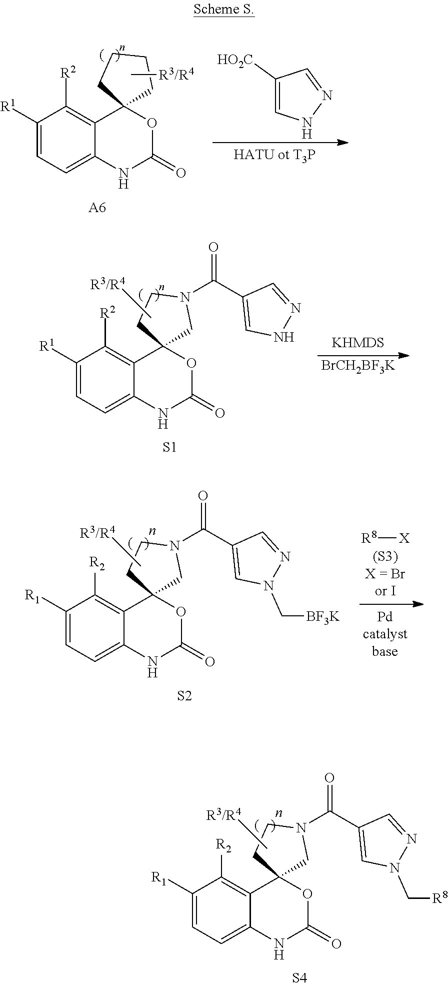

Scheme S illustrates a synthetic sequence for the preparation of a trifluoroborylalkyl-substituted pyrazole intermediate S2 from deprotected spirocarbamate synthon A6, which can undergo cross-coupling to produce S4. Amide formation by reacting spirocarbamate A6 and 4-pyrazolecarboxylic acid under conditions described previously can afford a pyrazolyl amide precursor S1 that can be further reacted in the presence of a suitable strong base, such as KHMDS, and potassium bromomethyltrifluoroborate to yield the alkyltrifluoroborate synthon S2 that can be cross-coupled with a suitable aryl halide (S3) in the presence of a palladium catalyst, like Cl2Pd(dppf), to afford S4, which can be carried on to compounds of this invention.

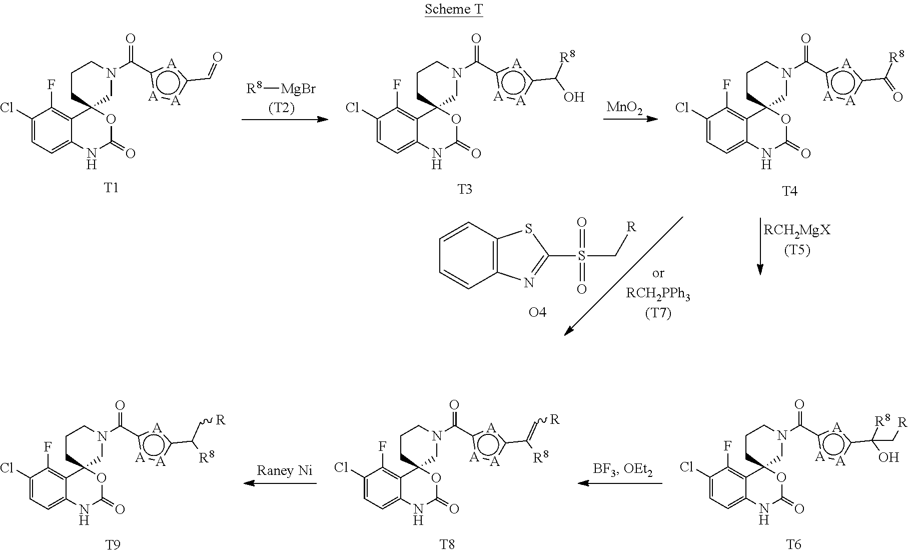

Scheme T illustrates a synthetic sequence to convert heteroaryl aldehydes T1 into branched alkyl heteroaryl congeners T6, T8 and T9. Reaction of a heteroaryl carboxaldehyde T1 with a suitable Grignard reagent (T2) yields a secondary alcohol T3 that can be oxidized to the corresponding ketone T4, under a variety of known methods, most preferably using manganese dioxide as an oxidant. Ketone T4 can be reacted with a second Grignard reagent (T5) to a tertiary alcohol (T6), which can serve as a compound of this invention. Alternatively, both tertiary alcohol T6 and ketone T4 can independently be converted to the corresponding vinyl heteroaryl amide T8. In the example of tertiary alcohol T6, elimination in the presence of a suitable acid or Lewis acid source can yield vinyl amide T8. The olefin moiety can also be synthesized in a single step from ketone T4, by treatment with a sulfone reagent (04) under Julia-Kociensky conditions or using a Wittig olefination reaction involving reaction with a phosphorus ylide reagent. Reduction of the olefin moiety in T8 can be achieved using a variety of conditions, most notably reaction with catalytic Raney Nickel to afford amide T9 which represents a general compound of this invention.

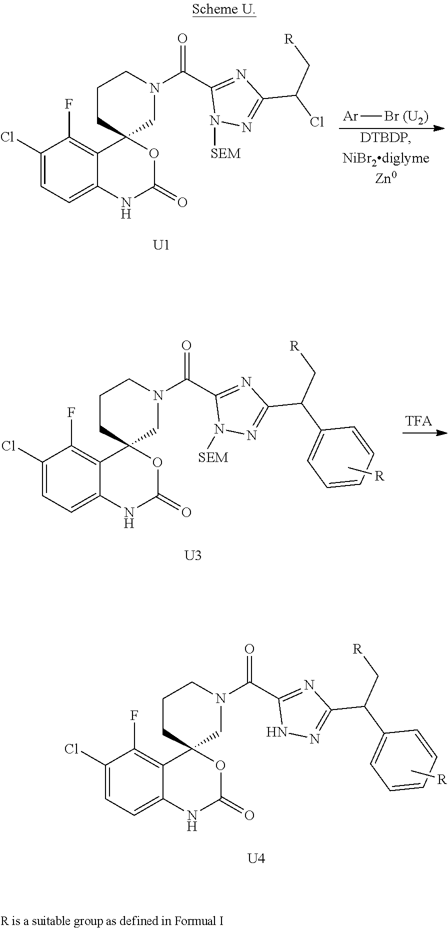

Scheme U illustrates an alternate method for synthesizing compounds of this invention (U4). Cross-coupling of a SEM-protected triazoloalkylchloride U1 with a suitable aryl bromide (U2) can be achieved using a Nickel-catalyzed reductive coupling procedure to afford a protected benzyltriazole U3. SEM deprotection in the presence of a strong acid, such as TFA, can give benzyl-substituted triazolyl amides such as U4.

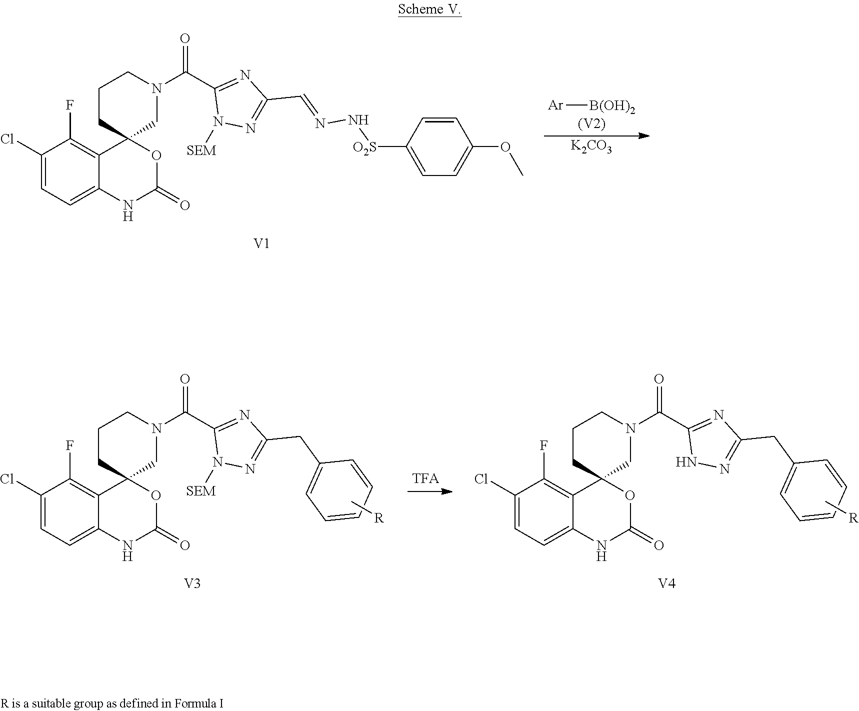

Scheme V illustrates an alternate method for synthesizing compounds of this invention (V4). Direct coupling of a suitable aryl boronic acid (V2) with a SEM-protected triazolosulfonylhydrazone V1 can be achieved in the presence of a suitable weak inorganic base, such as potassium carbonate, to afford a SEM-protected benzyltriazole V3. SEM deprotection as descibed above can give benzyltriazolyl amide V4.

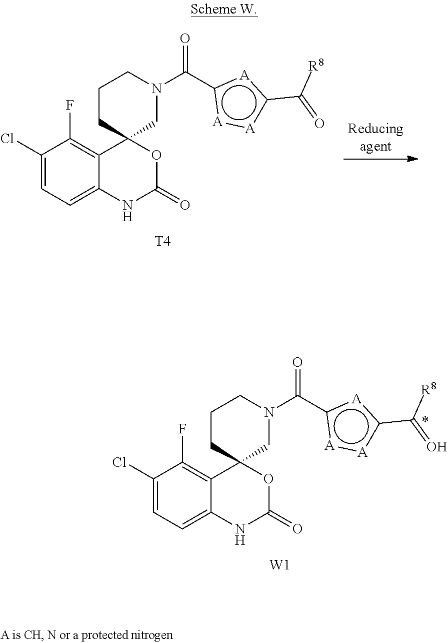

Scheme W illustrates a method for synthesizing benzylic alcohol compounds of this invention (WI). A variety of reducing agents, preferably, sodium borohydride, can successfully reduce intermediate ketones of type T4. In addition, a number of chiral reagents and biocatalytic ketoreductases can be employed to effect asymmetric reduction of the ketone moiety. All of which can be carried on further, as necessary, to compounds of this invention.

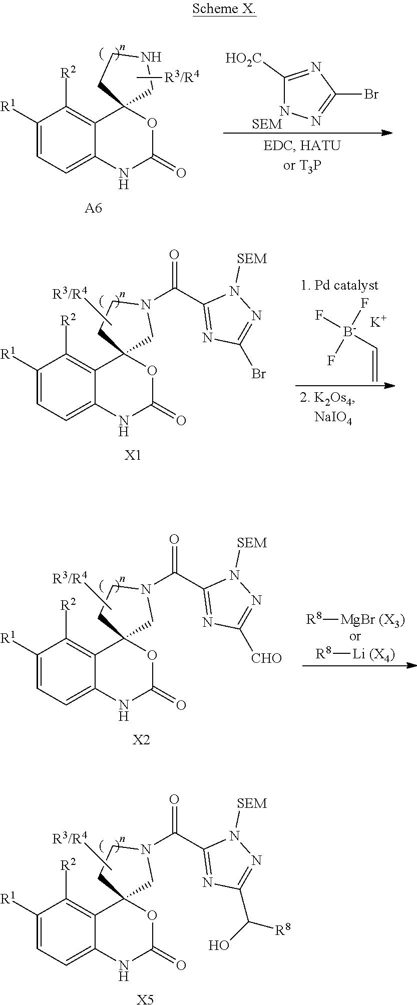

Scheme X illustrates an alternative method for synthesizing compounds of this invention (X5). Following procedures described above, amide coupling of A6 yields an intermediate X1 that can be reacted as described above in Scheme H to afford the triazolylcarboxaldehyde (X2). Reaction with an aryl Grignard (X3) or aryllithium (X4) reagent, the preparation which is known to those skilled in the art, gives a benzylic alcohol that can be carried on to compounds of this invention.

### Intermediates

### Intermediate A6-a

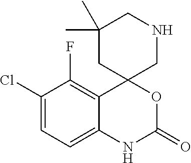 [SMILES: CC1(C)CNCC2(C1)OC(=O)Nc1ccc(Cl)c(F)c12]

### 6-Chloro-5-fluoro-5′,5′-dimethylspiro[benzo[d][1,3]oxazine-4,3′-piperidin]-2(1H)-one

Step 1: tert-Butyl 6-chloro-5-fluoro-5′,5′-dimethyl-2-oxo-1,2-dihydrospiro[benzo[d][1,3]oxazine-4,3′-piperidine]-1′-carboxylate: THF (55 mL) was added to a round-bottom flask containing tert-butyl (4-chloro-3-fluorophenyl)carbamate (2.21 g, 9.00 mmol) under N₂ atmosphere. The solution was cooled down to −78° C. To the stirring solution, ⁿBuLi (11 mL of a 2.5 M hexanes solution, 27.9 mmol) was added over 40 min. The reaction mixture was allowed to stir at −78° C. for an additional 45 min, at which time, a solution of LaCl₃·2LiCl (22.5 mL of a 0.6 M THF solution, 13.5 mmol) and tert-butyl 3,3-dimethyl-5-oxopiperidine-1-carboxylate (3.1 g, 13.5 mmol) was added at −78° C. over a period of 40 min. The reaction mixture was warmed to rt and stirred for 16 h. KO^{t}Bu (5.3 mL of a 1.7 M THF solution, 9.0 mmol) was added to the reaction mixture, and the resulting mixture was heated to 60° C. for 3 h. The mixture was cooled to rt, quenched with 1 M HCl and diluted with EtOAc. The layers were separated and aq. phase extracted with EtOAc. The combined organic layers were dried over MgSO₄, filtered and concentrated under reduced pressure. The crude residue was purified by silica gel chromatography (EtOAc/hexanes) to afford the title compound. LCMS [M+Na]⁺=421.1 (calcd. 421.1).

Step 2: 6-Chloro-5-fluoro-5,5′-dimethylspiro[benzo[d][1,3]oxazine-4,3′-piperidin]-2(1H)-one: HCl (25 mL of a 4 M dioxane solution, 100 mmol) was added to a round-bottom flask containing a suspension of tert-butyl 6-chloro-5-fluoro-5′,5′-dimethyl-2-oxo-1,2-dihydrospiro[benzo[d][1,3]oxazine-4,3′-piperidine]-1′-carboxylate (7.98 g, 20.0 mmol) in 1,4-dioxane (30 mL). The reaction mixture was heated to 90° C. and stirred vigorously for 12 h. The reaction was cooled to rt and concentrated under reduced pressure to give the crude title compound. The crude product was carried forward to the next step without further purification. LCMS [M+H]⁺=299.1 (calcd. 299.1).

The following compounds were prepared using procedures similar to those described for above using the appropriate starting materials.

| | | | Exact | |
|---|---|---|---|---|
| | | | Mass | Chiral |
| Example | Structure | Name | [M + H]⁺ | Column |
| A6-b | 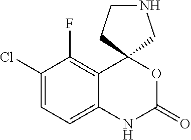 [SMILES: O=C1Nc2ccc(Cl)c(F)c2[C@@]2(CCNC2)O1] | (R)-6-Chloro-5- fluorospiro[benzo[d][1,3]oxazine- 4,3′-pyrrolidin]-2(1H)-one | Calc'd 257.1, found 257.0 | |
| A6-c | 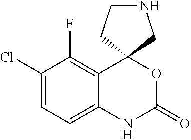 [SMILES: O=C1Nc2ccc(Cl)c(F)c2[C@]2(CCNC2)O1] | (S)-6-Chloro-5- fluorospiro[benzo[d][1,3]oxazine- 4,3′-pyrrolidin]-2(1H)-one | Calc'd 257.1, found 257.0 | |
| A6-d | 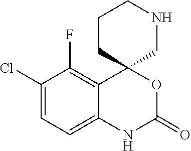 [SMILES: O=C1Nc2ccc(Cl)c(F)c2[C@@]2(CCCNC2)O1] | (R)-6-Chloro-5- fluorospiro[benzo[d][1,3]oxazine- 4,3′-piperidin]-2(1H)-one | Calc'd 271.1, found 271.0 | |
| A6-e | 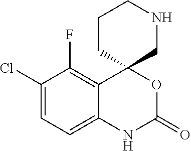 [SMILES: O=C1Nc2ccc(Cl)c(F)c2[C@]2(CCCNC2)O1] | (S)-6-Chloro-5- fluorospiro[benzo[d][1,3]oxazine- 4,3′-piperidin]-2(1H)-one | Calc'd 271.1, found 271.0 | |

### Intermediate B8-a

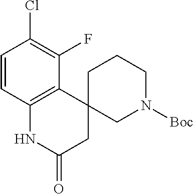 [SMILES: CC(C)(C)OC(=O)N1CCCC2(CC(=O)Nc3ccc(Cl)c(F)c32)C1]

### tert-Butyl 6′-chloro-5′-fluoro-2′-oxo-2′,3′-dihydro-1′H-spiro[piperidine-3,4′-quinoline]-1-carboxylate

Step 1. tert-Butyl 3-cyano-3-(2,6-difluorophenyl)propanoate: A solution of 2-(2,6-difluorophenyl) acetonitrile (10.0 g, 65.3 mmol) in THF (15 mL) was added dropwise to a solution of KHMDS (65.3 mL of a 1 M THF solution, 65.3 mmol) at −78° C. The resulting mixture was allowed to stir at −78° C. for 30 min, at which time, the reaction was warmed to 0° C. and allowed to stir for 30 min. A separate flask charged with tert-butyl 2-bromoacetate (12.7 g, 65.3 mmol) in THF (50 mL) was cooled to −48° C., and the above parent solution was added dropwise. The resulting mixture was slowly warmed up to 0° C. over 1 h, then quenched with satd. aq. NH₄Cl and extracted with EtOAc. The combined organics were washed with brine, dried (MgSO₄), filtered and evaporated under reduced pressure. The crude residue was purified by silica gel chromatography (EtOAc/hexanes) to give the title compound. LCMS [M+H]⁺=268.3 (calcd. 268.1).

Step 2. 1-(tert-Butyl) 6-methyl 3-cyano-3-(2,6-difluorophenyl)hexanedioate: To a mixture of tert-butyl 3-cyano-3-(2,6-difluorophenyl)propanoate (15.0 g, 56.1 mmol) and methyl acrylate (4.83 g, 56.1 mmol) was added N,N,N-trimethyl-1-phenylmethanaminium hydroxide (2.35 g, 5.61 mmol). The resulting mixture was stirred at rt for 10 min, at which time, the reaction was with EtOAc and washed with brine. The organic layer was dried (MgSO₄), concentrated, and the resulting crude residue was purified by silica gel chromatography (EtOAc/hexanes) to give the title compound. LCMS [M+H]⁺=354.3 (calcd. 354.2).

Step 3. tert-butyl 2-(3-(2,6-difluorophenyl)-6-oxopiperidin-3-yl)acetate: To a solution of 1-(tert-butyl) 6-methyl-3-cyano-3-(2,6-difluorophenyl)hexanedioate (18.0 g, 50.9 mmol) in MeOH (50 mL) was added cobalt(II) chloride hexahydrate (18.2 g, 76.0 mmol). The resulting mixture was allowed to stir at rt for 10 min, at which time, NaBH₄ (10.0 g, 264 mmol) was added in multiple portions. After 1.5 h, the reaction was diluted with water and extracted with EtOAc. The combined organics were dried (MgSO₄) and concentrated to the title compound that was carried on without further purification. LCMS [M+H]⁺=326.3 (calcd. 326.2).

Step 4. tert-Butyl 2-(1-benzyl-3-(2,6-difluorophenyl)piperidin-3-yl)acetate: To the solution of tert-butyl 2-(3-(2,6-difluorophenyl)-6-oxopiperidin-3-yl)acetate (11.0 g, 33.8 mmol) in THF (40 mL) at 0° C. was added borane-tetrahydrofuran complex (80 mL of a 1 M THE solution, 80 mmol), and the resulting mixture was warmed to rt and allowed to stir for 2 h. The reaction was cooled to 0° C., and quenched with AcOH. The resulting mixture was concentrated to afford a crude residue that was treated with ammonia (4.8 mL of a 7 M MeOH solution, 33.8 mmol). The mixture was concentrated, and the crude residue was diluted with THF (50 mL). AcOH (4.41 mL, 77 mmol) and benzaldehyde (5.86 mL, 57.8 mmol) were added, followed by sodium triacetoxyborohydride (12.3 g, 57.8 mmol), and the resulting mixture was stirred at rt. After 2 h, the reaction was diluted with EtOAc and washed with satd. aq. NaHCO₃, followed by brine. The organic layer was dried (MgSO₄), evaporated, and the crude residue was purified by silica gel chromatography (EtOAc/hexanes) to give the title compound. LCMS [M+H]⁺=402.5 (calcd. 402.2).

Step 5. 2-(1-Benzyl-3-(2,6-difluorophenyl)piperidin-3-yl)acetamide: A mixture of tert-butyl 2-(1-benzyl-3-(2,6-difluorophenyl)piperidin-3-yl)acetate (5.61 g, 14.0 mmol) and HCl (17.5 mL of a 4 M dioxane solution, 69.9 mmol) was stirred at rt. After 12 h, the reaction was concentrated, and the resulting crude residue was dissolved in DMF (50 mL). Ammonium chloride (1.64 g, 30.7 mmol) was added, followed by Et₃N (4.3 mL, 31 mmol) and HATU (6.38 g, 16.8 mmol), and the resulting reaction was stirred at rt. After 2 h, the reaction was filtered and concentrated to give the title compound as a crude residue that was carried on without purification. LCMS [M+H]⁺=345.4 (calcd. 345.2).

Step 6. tert-Butyl 5′-fluoro-2′-oxo-2′,3′-dihydro-1′H-spiro[piperidine-3,4′-quinoline]-1-carboxylate: The crude 2-(1-Benzyl-3-(2,6-difluorophenyl)piperidin-3-yl)acetamide (14.0 mmol) was dissolved in DMF (12 mL), and NaH (2.79 g, 69.9 mmol) was added portion-wise. The resulting mixture was heated to 130° C. for 30 min, at which time, the reaction was cooled to rt and neutralized with HCl. The reaction was concentrated, suspended in MeOH and filtered. To this filtrate was added Pd—C (1.49 g, 1.40 mmol), and the resulting mixture was degassed and stirred under an atmosphere of H₂. After 30 min, the reaction mixture was filtered through a pad of Celite®, and the filtrate was concentrated to afford a crude intermediate product that was redissolved in DMF (5 mL). Di-tert-butyl dicarbonate (3.2 mL, 14 mmol) was added, and the reaction was allowed to stir at rt. After 3 h, the reaction was diluted with EtOAc, washed with satd. aq. NaHCO₃ and brine, dried (MgSO₄), and concentrated. The crude residue was purified by silica gel chromatography (EtOAc/hexanes) to give the title compound. LCMS [M+H]⁺=335.4 (calcd. 335.2).

Step 7. tert-Butyl 6′-chloro-5′-fluoro-2′-oxo-2′,3′-dihydro-1′H-spiro[piperidine-3,4′-quinoline]-1-carboxylate: N-Chlorosuccinimide (359 mg, 2.69 mmol) was added to a solution of tert-butyl 5′-fluoro-2′-oxo-2′,3′-dihydro-1′H-spiro[piperidine-3,4′-quinoline]-1-carboxylate (900 mg, 2.69 mmol) in DMF (6 mL), and the resulting mixture was heated to 80° C. After 10 min, the reaction was diluted with EtOAc and washed with 1:1 mixture of satd. aq. NaHCO₃ and brine. The organic layer was dried (MgSO₄), concentrated, and the crude residue was purified by silica gel chromatography (EtOAc/hexanes) to give the title compound. LCMS [M+H]⁺=369.4 (calcd. 369.1).

The title compounds were separated by SFC (Instrument SFC-80 Method Column AS-H (250 mm*21 mm); Condition 35% EtOH Time (min); Flow Rate (mL/min) 50, Pressure (Bar) 120). The faster eluting isomer of the title compound was obtained (B8-a1): LCMS [M+H]⁺=369.4 (calcd. 369.1). The slower eluting isomer of the title compound was obtained (B8-a2): LCMS [M+H]⁺=369.4 (calcd. 369.1).

### Intermediate C4-a

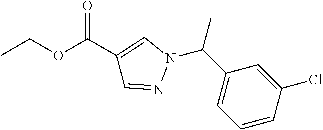 [SMILES: CCOC(=O)c1cnn(C(C)c2cccc(Cl)c2)c1]

### Ethyl (rac)-1-(1-(3-chlorophenyl)ethyl)-1H-pyrazole-4-carboxylate

To a mixture of 1-(1-bromoethyl)-3-chlorobenzene (4.40 g, 20.0 mmol) and Cs₂CO₃ (19.6 g, 60.1 mmol) in DMF (50 mL) was added ethyl 1H-pyrazole-4-carboxylate (3.09 g, 22.1 mmol). The mixture was stirred at 60° C. for 3 h. The mixture was partitioned between EtOAc and water, the layers were separated, and the aq. layer was extracted with EtOAc. The combined organic layers were washed with brine, dried over anhydrous Na₂SO₄, filtered and concentrated. The crude residue was purified by silica gel chromatography (EtOAc/petroleum ether) to give the title compound. LCMS [M+H]⁺=279.1, (calcd. 279.0). ¹H NMR (400 MHz, CDCl₃) δ 27.93 (d, J=4.2 Hz, 2H), 7.26 (d, J=4.5 Hz, 2H), 7.18 (s, 1H), 7.04-7.11 (m, 1H), 5.48 (q, J=7.1 Hz, 1H), 4.27 (q, J=7.1 Hz, 2H), 1.88 (d, J=7.1 Hz, 3H), 1.32 (t, J=7.1 Hz, 3H).

The following compounds were prepared using procedures similar to those described for above using the appropriate starting materials.

| | | | Exact Mass |
|---|---|---|---|
| Example | Structure | Name | [M + H]⁺ |
| C4-b | 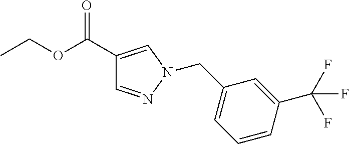 [SMILES: CCOC(=O)c1cnn(Cc2cccc(C(F)(F)F)c2)c1] | Ethyl 1-(3- (trifluoromethyl)benzyl)- 1H-pyrazole-4-carboxylate | 299.2 (calcd. 299.3) |
| C4-c | 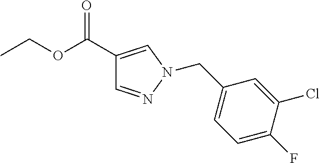 [SMILES: CCOC(=O)c1cnn(Cc2ccc(F)c(Cl)c2)c1] | Ethyl 1-(3-chloro-4- fluorobenzyl)-1H-pyrazole- 4-carboxylate | 285.0 (calcd. 284.0) |
| C4-d | 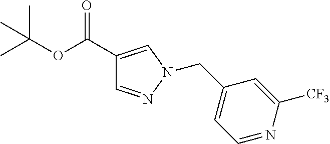 [SMILES: CC(C)(C)OC(=O)c1cnn(Cc2ccnc(C(F)(F)F)c2)c1] | Ethyl 1-((2- (trifluoromethyl)pyridin-4- yl)methyl)-1H-pyrazole-4- carboxylate | 328.1 (calcd. 328.3) |
| C4-e | 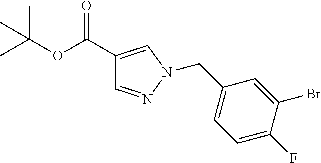 [SMILES: CC(C)(C)OC(=O)c1cnn(Cc2ccc(F)c(Br)c2)c1] | tert-Butyl 1-(3-bromo-4- fluorobenzyl)-1H-pyrazole- 4-carboxylate | 354.9 (calcd. 355.0) |
| C4-f | 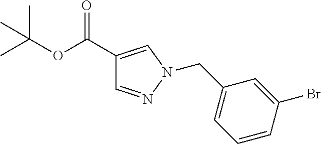 [SMILES: CC(C)(C)OC(=O)c1cnn(Cc2cccc(Br)c2)c1] | tert-Butyl 1-(3- bromobenzyl)-1H- pyrazole-4-carboxylate | 337.0 (calcd. 337.0) |
| C4-g | 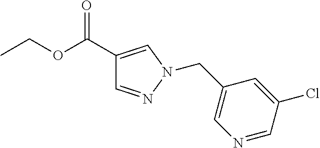 [SMILES: CCOC(=O)c1cnn(Cc2cncc(Cl)c2)c1] | Ethyl 1-((5-chloropyridin- 3-yl)methyl)-1H-pyrazole- 4-carboxylate | 266.1 (calcd. 266.1) |
| C4-h | 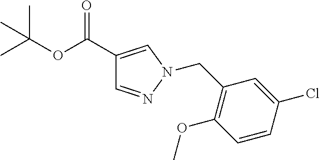 [SMILES: COc1ccc(Cl)cc1Cn1cc(C(=O)OC(C)(C)C)cn1] | tert-Butyl 1-(5-chloro-2- methoxybenzyl)-1H- pyrazole-4-carboxylate | 323.0 (calcd. 323.1) |

### Intermediate C5-a

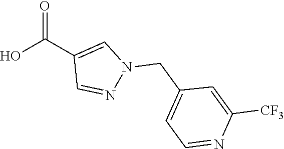 [SMILES: O=C(O)c1cnn(Cc2ccnc(C(F)(F)F)c2)c1]

### 1-Benzyl-1H-pyrazole-4-carboxylic acid

A 10 ml vial was charged with tert-butyl 1-((2-(trifluoromethyl)pyridin-4-yl)methyl)-1H-pyrazole-4-carboxylate (100 mg, 0.306 mmol) and TFA (230 μL, 39 mmol). The reaction was stirred at rt for 12 h. The crude reaction mixture was concentrated to dryness then triturated with ether to afford the title product, which was carried on without further purification. LCMS [M+H]⁺=272.0, calcd. 272.2

### Intermediate C5-b

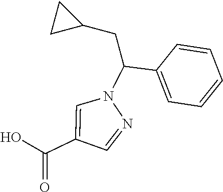 [SMILES: O=C(O)c1cnn(C(CC2CC2)c2ccccc2)c1]

1-(2-Cyclopropyl-1-phenylethyl)-1H-pyrazole-4-carboxylic acid Step 1: 2-Cyclopropyl-1-phenylethanone: To a solution of phenylmagnesium bromide (2.70 mL of a 3.0 M THF solution, 8.01 mmol) in THF (5 mL) was added 2-cyclopropylacetonitrile (500 mg, 6.16 mmol) in THF (2 mL) at 0° C. The resulting mixture was allowed to stir at 0° C. for 2 h, at which time, the reaction was quenched with 1 M HCl and extracted with EtOAc. The combined organic fractions were washed with brine, dried (Na₂SO₄), filtered and the solvent was evaporated under reduced pressure to give a crude residue that was purified by silica gel chromatography, (EtOAc/petroleum ether) to afford the title compound. ¹H NMR (500 MHz, CDCl₃) δ 7.93-7.99 (m, 2H), 7.53-7.61 (m, 1H), 7.42-7.50 (m, 2H), 2.89 (d, J=6.9 Hz, 2H), 1.10-1.26 (m, 1H), 0.55-0.66 (m, 2H), 0.15-0.25 (m, 2H).

Step 2: 2-Cyclopropyl-1-phenylethanol: To a solution of 2-cyclopropyl-1-phenylethanone (100 mg, 0.624 mmol) in MeOH (5 mL) was added NaBH₄ (35 mg, 0.94 mmol) at 0° C. The reaction was allowed to stir at 0° C. for 1 h, at which time, the mixture was concentrated to give a residue that was suspended in water and extracted with EtOAc. The combined organic fractions were dried (Na₂SO₄), filtered, and the solvent was evaporated under reduced pressure to afford the crude title compound that was carried on without purification. ¹H NMR (500 MHz, CDCl₃) δ 7.22-7.30 (m, 4H), 7.18 (dt, J=6.9, 2.2 Hz, 1H), 4.69 (t, J=6.6 Hz, 1H), 1.97 (br s, 1H), 1.52-1.64 (m, 2H), 0.57-0.67 (m, 1H), 0.27-0.43 (m, 2H), 0.03 (dq, J=9.2, 4.7 Hz, 1H), −0.12-−0.02 (m, 1H).

Step 3: Ethyl 1-(2-cyclopropyl-1-phenylethyl)-1H-pyrazole-4-carboxylate: Di-tert-butyl azodicarboxylate (170 mg, 0.740 mmol) was added to a stirred mixture of triphenylphosphine (155 mg, 0.592 mmol), 2-cyclopropyl-1-phenylethanol (80 mg, 0.493 mmol), ethyl 1H-pyrazole-4-carboxylate (69 mg, 0.49 mmol) in toluene (2 mL), and the resulting mixture was heated 80° C. for 2 h. The reaction was cooled to rt, diluted with water and extracted with EtOAc. The combined organic fractions were washed with brine, dried (Na₂SO₄), filtered, and the solvent was evaporated under reduced pressure to give a crude residue that was purified by preparative TLC (EtOAc/petroleum ether) to afford the title compound. LCMS [M+H]⁺=285.2 (calcd. 285.2).

Step 4: 1-(2-Cyclopropyl-1-phenylethyl)-1H-pyrazole-4-carboxylic acid: Lithium hydroxide hydrate (59 mg, 1.41 mmol) was added to a stirred solution of ethyl 1-(2-cyclopropyl-1-phenylethyl)-1H-pyrazole-4-carboxylate (80 mg, 0.28 mmol) in MeOH (1 mL) and water (0.2 mL), and the reaction was heated 40° C. for 2 h. The reaction was cooled to rt and concentrated to give a crude residue that was suspended in water and acidified with 1 M HCl to pH=6. The mixture was extracted with EtOAc, and the combined organic layers were concentrated to afford the crude title compound that was carried on without further purification. LCMS [M+H]⁺=257.1 (calcd. 257.1).

### Intermediate D5-a

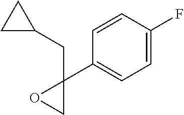 [SMILES: Fc1ccc(C2(CC3CC3)CO2)cc1]

### 2-(Cyclopropylmethyl)-2-(4-fluorophenyl)oxirane

Step 1: 2-Cyclopropyl-N-methoxy-N-methylacetamide: To a mixture of 2-cyclopropylacetic acid (5.0 g, 49.9 mmol) in DCM (20.0 mL) was added CDI (9.00 g, 55.5 mmol) at rt under N2. The mixture was stirred at rt for 1 h. Then N,O-dimethylhydroxylamine hydrochloride (5.50 g, 56.4 mmol) was added. The mixture was stirred at rt for another 15 h. The reaction was quenched with 1 N HCl, and the aq. layer was extracted with DCM. The combined organic layer was washed with 50% satd. aq. Na₂CO₃ and brine, dried with anhydrous Na₂SO₄, and concentrated to afford a crude residue that was purified by silica gel chromatography (EtOAc/petroleum ether) to give the title compound. LCMS [M+H]⁺=143.1. (calcd. 144.1). ¹H NMR (400 MHz, CDCl₃) δ 3.66 (d, J=1.9 Hz, 3H), 3.19 (d, J=1.9 Hz, 3H), 2.35 (br d, J=6.9 Hz, 2H), 1.03-1.15 (m, 1H), 0.51-0.59 (m, 2H), 0.12-0.21 (m, 2H).

Step 2: 2-Cyclopropyl-1-(4-fluorophenyl)ethenone: To a solution of 1-bromo-4-fluorobenzene (4.89 g, 27.9 mmol) in anhydrous THF (20 mL) at −78° C. under N₂ was added dropwise a solution of n-BuLi (11.2 mL, 27.9 mmol, 2.5 M in hexane). After stirring for 1 h at −78° C., a solution of 2-cyclopropyl-N-methoxy-N-methylacetamide (4.00 g, 27.9 mmol) in anhydrous THF (5 mL) was added dropwise. After addition, the reaction mixture was warmed to rt and stirred for 15 h. The reaction was quenched with satd. aq. NH₄Cl and extracted with EtOAc. The combined organic layers were washed with brine, dried over Na₂SO₄, and concentrated to afford a crude residue that was purified by silica gel chromatography (EtOAc/petroleum ether) to give the title compound. LCMS [M+H]⁺=178.2 (calcd. 178.1). ¹H NMR (400 MHz, CDCl₃) δ 8.00-8.11 (m, 2H), 7.15-7.29 (m, 2H), 2.92 (d, J=6.7 Hz, 2H), 1.03-1.19 (m, 1H), 0.49-0.62 (m, 2H), 0.10-0.26 (m, 2H).

Step 3: 2-(Cyclopropylmethyl)-2-(4-fluorophenyl)oxirane: Trimethylsulfonium iodide (1.15 g, 5.61 mmol) was suspended added in THF (15 mL). The mixture was cooled to 0° C., and potassium tert-butoxide (630 mg, 5.61 mmol) was added. The mixture was warmed to rt and stirred for 15 min. 2-Cyclopropyl-1-(4-fluorophenyl)ethanone (500 mg, 2.81 mmol) was added, and the resulting mixture continued stirring at rt for 30 h. The mixture was quenched with satd. aq. NH₄Cl and extracted with EtOAc. The combined organic layers were washed with brine, dried with anhydrous Na₂SO₄, and concentrated to give the title compound, which was used without further purification. LCMS [M+H]⁺=192.2 (calcd. 193.1).

### Intermediate E3-a

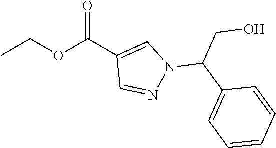 [SMILES: CCOC(=O)c1cnn(C(CO)c2ccccc2)c1]

### Ethyl 1-(2-hydroxy-1-phenylethyl)-1H-pyrazole-4-carboxylate

In a round bottom flask, to a mixture of ethyl 1H-pyrazole-4-carboxylate (1.50 g, 10.7 mmol) in 2-phenyloxirane (1.29 g, 10.7 mmol) was added yttrium(III) nitrate hexahydrate (0.031 mL, 0.214 mmol), and the resulting mixture was stirred at rt for 12 h. The reaction mixture was purified directly by silica gel chromatography (EtOAc/petroleum ether) to give the title compound. LCMS [M+H]⁺=261.1, (calcd. 261.1). ¹H NMR (500 MHz, CDCl₃) δ 8.01 (s, 1H), 7.81-7.86 (m, 1H), 7.34-7.39 (m, 3H), 7.17-7.23 (m, 2H), 5.42 (dd, J=8.4, 3.5 Hz, 1H), 4.45 (dd, J=12.2, 8.2 Hz, 1H), 4.24-4.30 (m, 2H), 4.13-4.17 (m, 1H), 1.32 (t, J=7.0 Hz, 3H).

The following compounds were prepared using procedures similar to those described for above using the appropriate starting materials.

| | | | Exact Mass |
|---|---|---|---|
| Example | Structure | Name | [M + H]⁺ |
| E3-b | 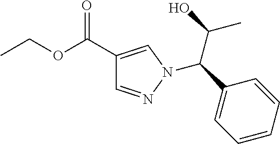 [SMILES: CCOC(=O)c1cnn([C@H](c2ccccc2)[C@H](C)O)c1] | (4R)-6-Chloro-5-fluoro-1′- [1-[(1R,2S)-2-hydroxy-1- phenyl-propyl]pyrazol-2- ium-4-carbonyl]spiro[1H- 3,1-benzoxazine-4,3′- piperidine]-2-one; formate | Calcd: 275.1 Found: 275.1 |
| E3-c | 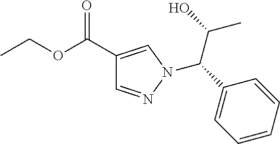 [SMILES: CCOC(=O)c1cnn([C@@H](c2ccccc2)[C@@H](C)O)c1] | (4R)-6-Chloro-5-fluoro-1′- [1-[(1S,2R)-2-hydroxy-1- phenyl-propyl]pyrazole-4- carbonyl]spiro[1H-3,1- benzoxazine-4,3′- piperidine]-2-one | Calcd: 275.1 Found: 275.0 |
| E3-d | 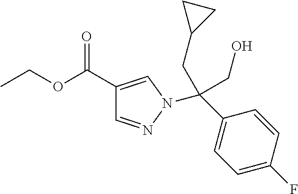 [SMILES: CCOC(=O)c1cnn(C(CO)(CC2CC2)c2ccc(F)cc2)c1] | Ethyl (R and S)-1-(1- cyclopropyl-2-(4- fluorophenyl)-3- hydroxypropan-2-yl)-1H- pyrazole-4-carboxylate | Calcd: 333.2 Found: 333.1 |

### Intermediate E4-a

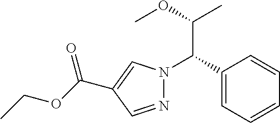 [SMILES: CCOC(=O)c1cnn([C@@H](c2ccccc2)[C@@H](C)OC)c1]

### Ethyl 1-((1S,2R)-2-methoxy-1-phenylpropyl)-1H-pyrazole-4-carboxylate

To ethyl 1-((1S,2R)-2-hydroxy-1-phenylpropyl)-1H-pyrazole-4-carboxylate (79 mg. 0.29 mmol) in DMF (2 ml) at 0° C. was added sodium hydride (11.5 mg, 0.288 mmol), and the resulting mixture was stirred for 15 min at 0° C. Iodomethane (18 μl, 0.29 mmol) was added, and the reaction mixture was warmed to rt for 4 h. The reaction was quenched with satd. aq. NH₄Cl and extracted with EtOAc. The combined organics were dried (MgSO₄) filtered and concentrated to afford a crude residue that was purified by silica gel chromatography (EtOAc/hexanes) to afford the title compound. LCMS [M+H]⁺=289.1 (calcd. 289.3). ¹H NMR (500 MHz, CDCl₃) δ 7.97 (d, J=22.3 Hz, 1H), 7.52 (d, J=6.9 Hz, 1H), 7.46-7.30 (m, 2H), 5.13 (dd, J=16.5, 7.2 Hz, OH), 4.85-4.10 (m, 1H), 3.26 (d, J=11.8 Hz, 2H), 1.35 (td, J=7.1, 3.0 Hz, 2H), 1.12 (dd, J=15.7, 6.2 Hz, 2H).

### Intermediate E4-b

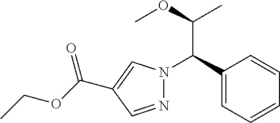 [SMILES: CCOC(=O)c1cnn([C@H](c2ccccc2)[C@H](C)OC)c1]

### Ethyl 1-((1R,2S)-2-methoxy-1-phenylpropyl)-1H-pyrazole-4-carboxylate

Intermediate E4-b was prepared using procedures similar to those as described for the preparation of intermediate E4-a above. LCMS [M+H]⁺=289.1 (calcd. 289.3).

### Intermediate F5-a

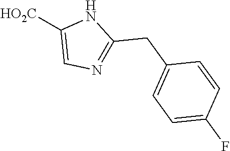 [SMILES: O=C(O)c1cnc(Cc2ccc(F)cc2)[nH]1]

### 2-(4-Fluorobenzyl)-1H-imidazole-5-carboxylic acid

Step 1: 2-(4-Fluorophenyl)-N-hydroxyacetimidamide: To a mixture of 2-(4-fluorophenyl)acetonitrile (1.0 g, 7.4 mmol) in MeOH (10 mL) was added hydroxylamine hydrochloride (617 mg, 8.88 mmol) and DIEA (3.2 mL, 19 mmol), and the reaction mixture was heated to 60° C. and allowed to stir overnight. The reaction was cooled to rt, and concentrated to afford a crude residue that as purified by silica gel chromatography (MeOH/DCM) to afford the title compound. LCMS [M+Na]⁺=168.9 (calcd. 169.1)

Step 2: Ethyl 2-(4-fluorobenzyl)-1H-imidazole-5-carboxylate: A solution of ethyl propiolate (660 μl, 6.54 mmol) and 2-(4-fluorophenyl)-N-hydroxyacetimidamide (1.00 g, 5.95 mmol) in EtOH was heated to 70° C. overnight. The reaction mixture was concentrated, at which time xylene was added, and the resulting mixture was heated to 200° C. for 30 min, and concentrated to afford a crude residue that was purified by silica gel chromatography (MeOH/DCM) to afford the title compound. LCMS [M+H]⁺=249.1 (calcd. 249.3).

Step 3: 2-(4-Fluorobenzyl)-1H-imidazole-5-carboxylic acid. The title compound was prepared following procedures similar to those described in Intermediate C5-b, Step 4.

### Intermediate G5-a

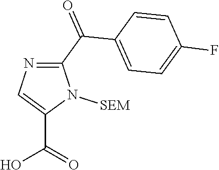 [SMILES: C[Si](C)(C)CCOCn1c(C(=O)O)cnc1C(=O)c1ccc(F)cc1]

### 2-(4-Fluorobenzoyl)-1-((2-(trimethylsilyl)ethoxy)methyl)-1H-imidazole-5-carboxylic acid

Step 1: Methyl 1-((2-(trimethylsilyl)ethoxy)methyl)-1H-imidazole-5-carboxylate: To a stirred solution of methyl 1H-imidazole-5-carboxylate (5.00 g, 39.6 mmol) and K₂CO₃ (11.0 g, 79.0 mmol) in ACN (50 mL) was added SEM-Cl (8.4 mL, 48 mmol), and the resulting mixture was allowed to stir at for 12 h. The reaction mixture was diluted with water and extracted with EtOAc. The combined organic extracts were washed with brine, dried (Na₂SO₄), filtered and concentrated to afford a crude residue that was purified by silica gel chromatography (EtOAc/petroleum ether) to give the title compound. LCMS [M+H]⁺=257.1 (calcd. 257.1).

Step 2: Methyl 2-(4-fluorobenzoyl)-1-((2-(trimethylsilyl)ethoxy)methyl)-1H-imidazole-5-carboxylate. 4-Fluorobenzoyl chloride (1.94 mL, 16.4 mmol) was added to a solution of methyl 1-((2-(trimethylsilyl)ethoxy)methyl)-1H-imidazole-5-carboxylate (3.50 g, 13.7 mmol) and TEA (2.3 mL, 16 mmol) in ACN (30 mL) at 0° C. The resulting mixture was warmed to rt and allowed to stir for 12 h. The reaction was concentrated to afford a crude residue that was purified by silica gel chromatography (EtOAc/petroleum ether) to give the title compound. LCMS [M-57]⁺=321.0 (calcd. 321.0).

Step 3: 2-(4-Fluorobenzoyl)-1-((2-(trimethylsilyl)ethoxy)methyl)-1H-imidazole-5-carboxylic acid. The title compound was prepared following procedures similar to those described in Intermediate C5-b, Step 4. LCMS [M+H]⁺=365.1 (calcd. 365.1).

### Intermediate H

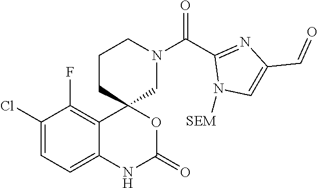 [SMILES: C[Si](C)(C)CCOCn1cc(C=O)nc1C(=O)N1CCC[C@@]2(C1)OC(=O)Nc1ccc(Cl)c(F)c12]

### (R)-2-(6-Chloro-5-fluoro-2-oxo-1,2-dihydrospiro[benzo[d][1,3]oxazine-4,3′-piperidine]-1′-carbonyl)-1-((2-(trimethylsilyl)ethoxy)methyl)-1H-imidazole-4-carbaldehyde

Step 1: Ethyl 4-bromo-1-((2-(trimethylsilyl)ethoxy)methyl)-1H-imidazole-2-carboxylate. To a solution of ethyl 4-bromo-1H-imidazole-2-carboxylate (3.00 g, 13.7 mmol) in DMF (40 mL) was added sodium hydride (657 mg, 16.4 mmol, 60% w/w dispersion in mineral spirits) at 0° C. for 30 min. (2-(Chloromethoxy)ethyl)trimethylsilane (3.43 g, 20.5 mmol) was added, and the resulting mixture was warmed to 30° C. for 10 h. The reaction was quenched with water and extracted with EtOAc. The organic layers were concentrated to afford a crude residue that was purified by silica gel chromatography (EtOAc/petroleum ether) to give the title compound. LCMS [M+H]⁺=349.1 (calcd. 349.0). ¹H NMR (CDCl₃, 400 MHz) δ 7.26 (s, 1H), 5.77 (s, 2H), 4.43 (q, J=7.1 Hz, 2H), 3.49-3.64 (m, 2H), 1.43 (t, J=7.1 Hz, 3H), 0.91-0.98 (m, 2H), 0.00 (s, 9H).

Step 2: Lithium 4-bromo-1-((2-(trimethylsilyl)ethoxy)methyl)-1H-imidazole-2-carboxylate. The title compound was prepared following procedures similar to those described in Intermediate C5-b, Step 4. LCMS (acid) [M+H]⁺=321.0 (calcd. 321.0).

Step 3: (R)-1′-(4-Bromo-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one. In a round bottom flask, to a solution of lithium 4-bromo-1-((2-(trimethylsilyl)ethoxy)methyl)-1H-imidazole-2-carboxylate (1.88 g, 5.73 mmol) and EDCI (2.20 g, 11.5 mmol) in pyridine (8 mL) was added (R)-6-chloro-5-fluorospiro[benzo[d][1,3]oxazine-4,3′-piperidin]-2(1H)-one (2.53 g, 6.88 mmol), and the reaction mixture was heated at 30° C. for 16 h. The reaction was quenched with water and extracted with EtOAc. The organic layers were washed with 1M HCl and concentrated to afford a crude residue that was purified by silica gel chromatography (EtOAc/petroleum ether) to give the title compound. LCMS [M+H]⁺=573.07 (calcd 573.0).

Step 4: (R)-6-chloro-5-fluoro-1′-(1-((2-(trimethylsilyl)ethoxy)methyl)-4-vinyl-1H-imidazole-2-carbonyl)spiro[benzo[d][1.3]oxazine-4,3′-piperidin]-2(1H)-one. To a solution of (R)-1′-(4-bromo-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one (2.00 g, 3.48 mmol) in EtOH (20 mL) was added potassium vinyltrifluoroborate (607 mg, 4.53 mmol), Pd(dppf)Cl₂ (510 mg, 6.97 mmol) and TEA (1.46 mL, 10.5 mmol), and the mixture was stirred at 90° C. for 6 h. The reaction mixture was cooled to rt and partitioned between EtOAc and water. The layers were separated, and the organic layer was concentrated to afford a crude residue that was purified by silica gel chromatography (EtOAc/petroleum ether) to give the title compound. LCMS [M+H]⁺=521.2 (calcd. 521.3).

Step 5: (R)-2-(6-chloro-5-fluoro-2-oxo-1,2-dihydrospiro[benzo[d][1,3]oxazine-4,3′-piperidin]-1′-ylcarbonyl)-1-((2-(trimethylsilyl)ethoxy)methyl)-1H-imidazole-4-carbaldehyde. To a solution of (R)-6-chloro-5-fluoro-1′-(1-((2-(trimethylsilyl)ethoxy)methyl)-4-vinyl-1H-imidazole-2-carbonyl)spiro[benzo[d][1,3]oxazine-4,3′-piperidin]-2(1H)-one (1.30 g, 2.50 mmol) in acetone (5 mL) and water (5 mL) was added potassium osmate(VI) dihydrate (37 mg, 0.10 mmol), and the resulting mixture was stirred at 0° C. for 10 min. Sodium periodate (2.14 g, 9.98 mmol) was added, and the reaction was stirred at rt for 12 h. The reaction mixture was purified directly by silica gel chromatography (EtOAc/petroleum ether) to give the title compound. LCMS [M+H]⁺=523.2 (calcd. 523.2).

### Intermediate H4-a

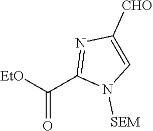 [SMILES: CCOC(=O)c1nc(C=O)cn1COCC[Si](C)(C)C]

### Ethyl 4-formyl-1-((2-(trimethylsilyl)ethoxy)methyl)-1H-imidazole-2-carboxylate

Step 1: Ethyl 4-bromo-1-((2-(trimethylsilyl)ethoxy)methyl)-1H-imidazole-2-carboxylate: To a solution of ethyl 4-bromo-TH-imidazole-2-carboxylate (1.00 g, 4.57 mmol) in DMF (8 mL) was added sodium hydride (219 mg, 5.48 mmol, 60% wt. dispersion in mineral spirits) in several portions at 0° C. After 30 min, (2-(chloromethoxy)ethyl)trimethylsilane (1.14 g, 6.85 mmol) was added, the resulting mixture was allowed to warm to rt. The reaction was quenched with water and extracted with EtOAc. The combined organic layers were concentrated and purified by silica gel chromatography (EtOAc/petroleum ether) to afford the title compound. LCMS [M+H]⁺=348.9/350.9 (calcd. 349.1, 351.1).

Step 2: Ethyl 1-((2-(trimethylsilyl)ethoxy)methyl)-4-vinyl-1H-imidazole-2-carboxylate: To a solution of ethyl 4-bromo-1-((2-(trimethylsilyl)ethoxy)methyl)-1H-imidazole-2-carboxylate (1.10 g, 3.15 mmol) in EtOH (15 mL) was added potassium vinyltrifluoroborate (548 mg, 4.09 mmol), Pd(dppf)Cl₂ (691 mg, 0.945 mmol) and TEA (1.3 mL, 9.5 mmol), The resulting mixture was heated to 90° C. for 12 h. The reaction was cooled to rt, diluted with water, and extracted with EtOAc. The combined organic layers were concentrated and purified by silica gel chromatography (EtOAc/petroleum ether) to afford the title compound. LCMS [M+H]⁺=297.2 (calcd. 297.2).

Step 3: Ethyl 4-formyl-1-((2-(trimethylsilyl)ethoxy)methyl)-1H-imidazole-2-carboxylate: To a solution of ethyl 1-((2-(trimethylsilyl)ethoxy)methyl)-4-vinyl-1H-imidazole-2-carboxylate (600 mg, 2.02 mmol) in acetone (5 mL) and water (5 mL) was added potassium osmate(VI) dihydrate (32 mg, 0.086 mmol) at 0° C. After 10 min, NaIO₄ (864 mg, 4.04 mmol) was added, the resulting mixture was warmed to rt and allowed to stir overnight. The reaction was diluted with water and extracted with EtOAc. The combined organics were concentrated and purified by silica gel chromatography (EtOAc/petroleum ether) to afford the title compound. LCMS [M+H]⁺=299.2 (calcd. 299.1).

### Intermediate I4-a

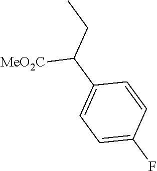

### Methyl 2-(4-fluorophenyl) butanoate

Step 1: 2-(4-Fluorophenyl)butanoic acid: To a solution of 2-(4-fluorophenyl) acetic acid (1.00 g, 6.49 mmol) in THF (10 mL) was added LDA (7.10 mL, 14.3 mmol, 2 M THF solution) at −78° C. The resulting mixture was warmed to rt over 30 min, at which time, iodoethane (1.21 g, 7.79 mmol) was added in a single portion, and the reaction was stirred at rt for 4 h. The reaction was concentrated, and the resulting crude residue was partitioned between EtOAc and water. The layers were separated, and the aq. layer was acidified by addition of 1 M HCl and extracted with EtOAc. The combined organics were dried (Na₂SO₄) and concentrated to give the title compound that was carried on without further purification. ¹H NMR (500 MHz, CD₃OD) δ 7.31-7.35 (m, 2H), 7.03-7.07 (m, 2H), 3.59 (s, 1H), 2.00-2.02 (m, 1H), 1.69-1.78 (m, 1H), 0.89 (t, J=7.4 Hz, 3H).

Step 2: Methyl 2-(4-fluorophenyl) butanoate. (Diazomethyl)trimethylsilane (4.5 mL, 9.1 mmol, 2.0 M toluene solution) was added to a stirred solution of 2-(4-fluorophenyl)butanoic acid (1.1 g, 6.0 mmol) in DCM (7.5 mL) and MeOH (1.5 ml) at 0° C. and the resulting mixture was stirred at 0° C. for 1 h. The reaction was quenched by addition of 5% aq. AcOH, followed by neutralization by addition to satd. aq. NaHCO₃. The mixture was extracted with DCM, and the combined organic fractions were washed with brine, dried (Na₂SO₄), filtered and concentrated to afford a crude residue that was purified by silica gel chromatography (EtOAc/petroleum ether) to give the title compound. ¹H NMR (400 MHz, CDCl₃) δ 7.27-7.29 (m, 1H), 7.24-7.27 (m, 1H), 6.97-7.03 (m, 2H), 4.12 (q, J=7.3 Hz, 1H), 3.66 (s, 3H), 3.36-3.50 (m, 1H), 2.03-2.12 (m, 1H), 0.88 (t, J=7.4 Hz, 3H).

### Intermediate K4-a

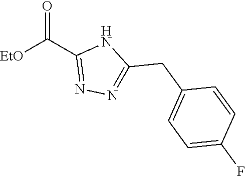 [SMILES: CCOC(=O)c1nnc(Cc2ccc(F)cc2)[nH]1]

### Ethyl 5-(4-fluorobenzyl)-4H-1,2,4-triazole-3-carboxylate

The title compound was prepared following procedures similar to those described above for Intermediate K5-a, Steps 1-3. ¹H NMR (500 MHz, CD₃OD) δ 7.30 (dd, J=8.5, 5.4 Hz, 2H), 6.99-7.10 (m, 2H), 4.41 (q, J=7.1 Hz, 2H), 4.16 (s, 2H), 1.39 (t, J=7.2 Hz, 3H).

### Intermediate K5-a

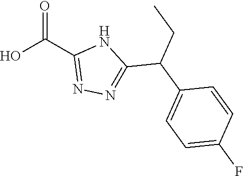 [SMILES: CCC(c1ccc(F)cc1)c1nnc(C(=O)O)[nH]1]

### 5-(1-(4-Fluorophenyl)propyl)-4H-1,2,4-triazole-3-carboxylic acid

Step 1: 2-(4-Fluorophenyl)butanehydrazide: Hydrazine hydrate (781 mg, 15.3 mmol) was added to a stirred solution of methyl 2-(4-fluorophenyl)butanoate (300 mg, 1.53 mmol) in EtOH (3 mL), and the resulting mixture was heated to 80° C. for 12 h. The reaction was cooled to 0° C., and filtered to afford the crude title compound that was carried on without further purification. LCMS [M+H]⁺=197.1 (calcd. 197.1).

Step 2: (Z)-Ethyl 2-amino-2-(2-(2-(4-fluorophenyl)butanoyl)hydrazono)acetate. Ethyl 2-ethoxy-2-iminoacetate (148 mg, 1.02 mmol) was added to a stirred solution of 2-(4-fluorophenyl)butanehydrazide (100 mg, 0.510 mmol) in EtOH (1 mL), and the resulting mixture was heated to 80° C. for 12 h. The reaction was cooled to rt and concentrated to afford the title compound that was carried on without further purification. LCMS [M+H]⁺=296.2 (calcd. 296.1).

Step 3: Ethyl 5-(1-(4-fluorophenyl)propyl)-4H-1,2,4-triazole-3-carboxylate. 4A Molecular sieves were added to a stirred solution of (Z)-ethyl 2-amino-2-(2-(2-(4-fluorophenyl)butanoyl)hydrazono)acetate (110 mg, 0.372 mmol) in xylene (1.5 mL), and the resulting mixture was heated to 150° C. for 24 h. The reaction was cooled to rt and concentrated to afford a crude residue that was purified by preparative TLC (EtOAc/petroleum ether) to give the title compound. ¹H NMR (500 MHz, CD₃OD) δ 7.31-7.38 (m, 2H), 7.00-7.08 (m, 2H), 4.36-4.48 (m, 2H), 4.09 (br t, J=7.9 Hz, 1H), 2.21-2.32 (m, 1H), 2.03-2.11 (m, 1H), 1.40 (t, J=7.2 Hz, 3H), 0.83-0.95 (m, 3H). LCMS [M+H]⁺=278.2 (calcd. 278.1).

Step 6: 5-(1-(4-Fluorophenylpropyl)-4H-1,2,4-triazole-3-carboxylic acid. The title compound was prepared following procedures similar to those described above for Intermediate C5-b, Step 4. LCMS [M+H]⁺=250.1 (calcd. 250.1).

### Intermediate L2-a

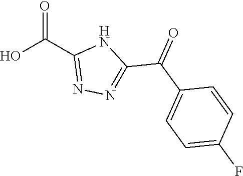 [SMILES: O=C(O)c1nnc(C(=O)c2ccc(F)cc2)[nH]1]

### 5-(4-Fluorobenzoyl)-4H-1,2,4-triazole-3-carboxylic acid

Step 1: Ethyl 5-(4-fluorobenzoyl)-4H-12,4-triazole-3-carboxylate: Potassium permanganate (1.08 g, 6.82 mmol) was added to a stirred solution of ethyl 5-(4-fluorobenzyl)-4H-1,2,4-triazole-3-carboxylate (850 mg, 3.41 mmol) in DCM (15 mL), and the resulting mixture was allowed to stir at rt. After 12 h, the reaction was diluted with 1 M HCl and extracted with EtOAc. The layers were separated, and the organic layer was concentrated to afford a crude residue that was purified by silica gel chromatography (EtOAc/petroleum ether) to give the title compound. LCMS [M+H]⁺=264.1 (calcd. 264.1).

Step 2: 5-(4-Fluorobenzoyl)-4H-1,2,4-triazole-3-carboxylic acid. The title compound was prepared following procedures similar to those described above for Intermediate C5-b, Step 4. LCMS [M+H]⁺=236.0 (calcd. 236.0).

### Intermediate L2-b

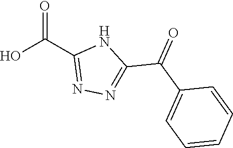 [SMILES: O=C(O)c1nnc(C(=O)c2ccccc2)[nH]1]

5-Benzoyl-4H-1,2,4-triazole-3-carboxylic acid. The title compound was prepared following procedures similar to those described above for Intermediate L2-a. LCMS [M+H]⁺=218.0 (calcd. 218.1).

### Intermediate M9-a

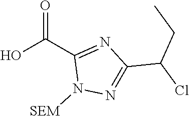 [SMILES: CCC(Cl)c1nc(C(=O)O)n(COCC[Si](C)(C)C)n1]

### 3-(1-Chloropropyl)-1-((2-(trimethylsilyl)ethoxy)methyl)-1H-1,2,4-triazole-5-carboxylic acid

Step 1: tert-Butyl 2-(2-ethoxy-1-imino-2-oxoethyl)hydrazine-1-carboxylate. Boc-hydrazide (19.8 g, 150 mmol) was combined with ethyl 2-amino-2-thioxoacetate (20.0 g, 150 mmol) in EtOH (80 mL), and the resulting mixture was allowed to stir at rt. After 23 h, the reaction was filtered, and the filter cake was washed with EtOH and dried under vacuum to afford the title compound. LCMS [M+H]⁺=232.15 (calcd. 232.3).

Step 2: Ethyl 5-((benzyloxy)methyl)-4H-1,2,4-triazole-3-carboxylate. A solution of tert-butyl 2-(2-ethoxy-1-imino-2-oxoethyl)hydrazine-1-carboxylate (45.0 g, 195 mmol) and benzyloxyacetyl chloride (9.3 ml, 59 mmol) in pyridine (100 ml) was heated to 105° C. After 5 h, the reaction was cooled to rt, diluted with EtOAc and acidified with 1 M HCl. The mixture was neutralized by the addition of satd. aq. NaHCO₃, and the layers were separated. The organic layer was dried (Na₂SO₄) and concentrated to afford a crude residue that was purified by silica gel chromatography (EtOAc/Hexanes) to give the title compound. LCMS [M+H]⁺=262.0 (calcd. 262.3).

Step 3: Ethyl 5-(hydroxymethyl)-4-((2-(trimethylsilyl)ethoxy)methyl)-4H-1,2,4-triazole-3-carboxylate. Sodium hydride (2.04 g, 50.9 mmol, 60% w/w dispersion in mineral spirits) was added in several portions to a stirred solution that was maintained under positive N₂ pressure of ethyl 5-((benzyloxy)methyl)-4H-1,2,4-triazole-3-carboxylate (12.1 g, 46.3 mmol) in THF (93 ml) at 0° C. After 15 min, SEM-Cl (9.9 ml, 56 mmol) was added dropwise, and the resulting mixture was allowed to warm to rt and stirred overnight at rt. The reaction was quenched by the addition of ice in several portions, at which point, the mixture was diluted with water and extracted with EtOAc. The combined organic layers were dried (Na₂SO₄), filtered and concentrated to afford the crude product that was dissolved in EtOH (120 mL). The reaction vessel was evacuated and flushed three times with nitrogen, at which point, palladium hydroxide on carbon (51 g, 73 mmol) was added, and the mixture was degassed again, as described above, followed by a single degassing cycle that replaced the reaction atmosphere with hydrogen. The reaction mixture was allowed to stir under hydrogen (1 atm) at rt for 48 h, at which point, the reaction was filtered through Celite®, and the filtrate was concentrated to afford the crude title compound that was carried on without further purification. LCMS [M+H]⁺=302.1 (calcd. 302.4).

Step 4: Ethyl 5-(1-hydroxypropyl)-4-((2-(trimethylsilyl)ethoxy)methyl)-4H-1,2,4-triazole-3-carboxylate. DMP (3.93 g, 9.26 mmol) was added to a stirred solution of ethyl 5-(hydroxymethyl)-4-((2-(trimethylsilyl)ethoxy)methyl)-4H-1,2,4-triazole-3-carboxylate (1.86 g, 6.17 mmol) in DCM (19 ml), and the resulting mixture was allowed to stir at rt. After 1.5 h, the reaction was quenched with satd. aq. NaHCO₃ and satd. aq. Na₂S₂O₃. The layers were separated and the aq. layer was extracted with DCM. The combined organic layers were washed with satd. aq. NaHCO₃, dried (MgSO₄) and concentrated to afford a crude residue that was dissolved in toluene (19 mL) and cooled to 0° C. Diethylzinc (7.2 ml of a 15% toluene solution, 8.02 mmol) was added dropwise, and the reaction continued to stir at 0° C. After 1 h, the reaction was quenched with satd. aq. NH₄Cl, warmed to rt, diluted with water and EtOAc and allowed to stir at rt overnight. The layers were separated and the aq. layer was extracted with EtOAc. The combined organic layers were washed with brine, dried (MgSO₄), filtered and concentrated to afford a crude residue that was purified by silica gel chromatography (EtOAc/hexanes) to give the title compound. LCMS [M+H]⁺=330.2 (calcd. 330.5).

Step 5: Ethyl 5-(1-chloropropyl)-4-((2-(trimethylsilyl)ethoxy)methyl)-4H-1,2,4-triazole-3-carboxylate. Triphenylphosphine (3.50 g, 13.4 mmol) was added to a stirred solution of ethyl 5-(1-hydroxypropyl)-4-((2-(trimethylsilyl)ethoxy)methyl)-4H-1,2,4-triazole-3-carboxylate (4.00 g, 12.1 mmol) and carbon tetrachloride (18.7 g, 121 mmol) in DCM (40 mL) at rt. After 4 h, the reaction was concentrated to afford a crude residue that was purified by silica gel chromatography (EtOAc/hexanes) to the title compound. LCMS [M+H]⁺ 348.2 (calcd. 348.2).

Step 6: 3-(1-Chloropropyl)-1-((2-(trimethylsilyl)ethoxy)methyl)-1H-1,2,4-triazole-5-carboxylic acid. The title compound was prepared following procedures similar to those described above for Intermediate C5-b, Step 4. [M-CO₂+H]⁺=276.2 (calcd. 276.1).

### Intermediate M-a

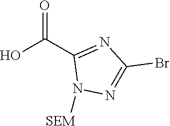 [SMILES: C[Si](C)(C)CCOCn1nc(Br)nc1C(=O)O]

### 3-Bromo-1-((2-(trimethylsilyl)ethoxy)methyl)-1H-1,2,4-triazole-5-carboxylic acid

The title compound was prepared from ethyl 3-bromo-1H-1,2,4-triazole-5-carboxylate following procedures similar to those described above for Intermediate M9-a, Step 3 and Intermediate C5-b, Step 4. LCMS [M+H]⁺=322.2 (calcd. 322.0).

### Intermediate O5-a

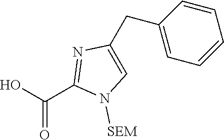 [SMILES: C[Si](C)(C)CCOCn1cc(Cc2ccccc2)nc1C(=O)O]

### 4-Benzyl-1-((2-(trimethylsilyl)ethoxy)methyl)-1H-imidazole-2-carboxylic acid

Step 1: (Z)-Ethyl 4-((2-((4-methoxyphenyl)sulfonyl)hydrazono)methyl)-1-((2-(trimethylsilyl) ethoxy)methyl)-1H-imidazole-2-carboxylate: To a solution of Intermediate H4-a (400 mg, 1.340 mmol) in MeOH (6 mL) was added 4-methoxybenzenesulfonyl hydrazide (271 mg, 1.34 mmol). The reaction mixture stirred at rt for 2 h, and concentrated to afford the crude title compound that was carried on without purification. LCMS [M+H]⁺=483.1 (calcd. 483.2).

Step 5: Ethyl 4-benzyl-1-((2-(trimethylsilyl)ethoxy)methyl)-1H-imidazole-2-carboxylate: To a stirred mixture of (Z)-ethyl 4-((2-((4-methoxyphenyl)sulfonyl)hydrazono)methyl)-1-((2-(trimethylsilyl)ethoxy)methyl)-1H-imidazole-2-carboxylate (250 mg, 0.518 mmol) and phenylboronic acid (95 mg, 0.78 mmol) in dioxane (5 mL) was added potassium carbonate (107 mg, 0.777 mmol). The reaction mixture was heated to 100° C. and allowed to stir at 100° C. for 12 h. The mixture was cooled to rt and diluted with water and EtOAc. The combined organic layers were concentrated and purified by preparative TLC (EtOAc:petroleum ether) to afford the title compound. LCMS [M+H]⁺=361.3 (calcd. 361.2).

Step 6: 4-Benzyl-1-((2-(trimethylsilyl)ethoxy)methyl)-1H-imidazole-2-carboxylic acid: The title compound was prepared following procedures similar to those described in Intermediate C5-b, Step 4. LCMS [M+H]⁺=333.2 (calcd. 333.2).

### Intermediate P3-a

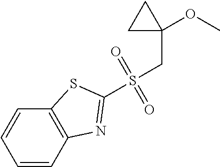 [SMILES: COC1(CS(=O)(=O)c2nc3ccccc3s2)CC1]

### 2-(((1-Methoxycyclopropyl)methyl)sulfonyl)benzo[d]thiazole

Step 1: 2-(((1-Methoxycyclopropyl)methyl)thio)benzo[d]thiazole: DEAD (930 μL, 5.87 mmol) was added to a solution of (1-methoxycyclopropyl)methanol (300 mg, 2.94 mmol), benzo[d]thiazole-2-thiol (590 mg, 3.52 mmol) and triphenylphosphine (925 mg, 3.52 mmol) in THF (5 mL) at 0° C. The mixture was stirred at 0° C. for 2 h, at which time, the reaction was diluted with water and extracted with EtOAc. The combined organic layers were concentrated, and purification by preparative TLC (EtOAc/petroleum ether) to give the title compound. LCMS [M+H]⁺=252.0 (calcd. 252.0).

Step 2: 2-(((1-Methoxycyclopropyl)methyl)sulfonyl)benzo[d]thiazole: To a solution of 2-(((1-methoxycyclopropyl)methyl)thio)benzo[d]thiazole (350 mg, 1.39 mmol) in EtOH (5 mL) was added ammonium molybdate tetrahydrate (172 mg, 0.139 mmol) and H₂O₂ (5.7 mL, 56 mmol, 30% v/v aq. solution). The resulting mixture was allowed to stir at rt. The reaction was concentrated, and the resulting crude residue was purified by preparative TLC (EtOAc/petroleum ether) to give the title compound. LCMS [M+H]⁺=284.0 (calcd. 284.0).

### Intermediate Q4-a

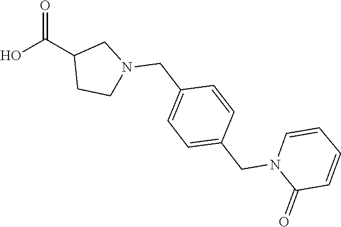 [SMILES: O=C(O)C1CCN(Cc2ccc(Cn3ccccc3=O)cc2)C1]

### 1-(4-((2-Oxopyridin-1(2H)-yl)methyl)benzyl)pyrrolidine-3-carboxylic acid

Step 1: Methyl pyrrolidine-3-carboxylate hydrochloride: To a mixture of pyridin-2(1H)-one (721 mg, 7.58 mmol) and TBAI (280 mg, 0.758 mmol) in THF (30 mL) was added NaH (303 mg, 7.58 mmol, 60% w/w dispersion in mineral spirits) at 0° C. The resulting mixture was stirred at rt for 30 min, at which time, 1,4-bis(bromomethyl)benzene (2.00 g, 7.58 mmol) was added. After 2 h, the reaction mixture was quenched with satd. aq. NH₄Cl and extracted with EtOAc. The combined organic layers were washed with brine, dried over Na₂SO₄, filtered, and concentrated to afford a crude residue that was by purified silica gel chromatography (EtOAc/hexane) to give the title compound. LCMS [M+H]⁺=278.0, (calcd. 278.0). ¹H NMR (400 MHz, CDCl₃): δ 7.42-7.20 (m, 6H), 6.67-6.59 (m, 1H), 6.21-6.13 (m, 1H), 5.15 (s, 2H), 4.48 (s, 2H).

Step 2: Methyl 1-(4-((2-oxopyridin-1(2H)-yl)methyl)benzyl)pyrrolidine-3-carboxylate: To a mixture of methyl pyrrolidine-3-carboxylate hydrochloride (119 mg, 0.719 mmol) in DMF (2 mL) was added NaH (60 mg, 1.51 mmol, 60% wt dispersion in mineral spirits) at 0° C. The mixture was stirred warmed to rt for 30 min, at which time, a solution of 1-(4-(bromomethyl)benzyl)pyridin-2(1H)-one (200 mg, 0.719 mmol) in DMF (0.2 mL) was added, and the resulting mixture was stirred at rt for 1 h. The reaction was quenched with satd. aq. NH₄Cl and extracted with EtOAc. The combined organic layers were washed with brine, dried over Na₂SO₄, filtered, and concentrated to give the crude title compound. LCMS [M+H]⁺ 327.3 (calcd. 327.16).

Step 3: 1-(4-((2-oxopyridin-1(2H)-yl)methyl)benzyl)pyrrolidine-3-carboxylic acid: To a mixture of methyl 1-(4-((2-oxopyridin-1(2H)-yl)methyl)benzyl)pyrrolidine-3-carboxylate (300 mg, 0.643 mmol) in DMF (1 mL) was added lithium hydroxide hydrate (135 mg, 3.22 mmol) in water (0.2 mL), and the resulting mixture was stirred at rt for 2 h. The reaction was acidified to pH 5 via 1M HCl and purified by reverse phase HPLC (ACN/water with 0.05% TFA modifier) to give the title compound. LCMS [M+H]⁺=313.2, (calcd. 313.2). 1H-NMR (400 MHz, DMSO-d₆): δ 10.10 (s, 1H), 7.86-7.79 (m, 1H), 7.53-7.41 (m, 3H), 7.37 (d, J=7.9 Hz, 2H), 6.44 (d, J=9.1 Hz, 1H), 6.31-6.23 (m, 111), 5.14 (s, 2H), 4.36 (d, J=12.3 Hz, 2H), 3.41 (s, 2H), 3.18 (s, 2H), 2.72-2.55 (m, 1H), 2.40-1.94 (m, 2H).

The following compounds were prepared using procedures similar to those described for above using the appropriate starting materials.

| | | | Exact Mass |
|---|---|---|---|
| Intermediate | Structure | Name | [M + H]⁺ |
| Q4-b | 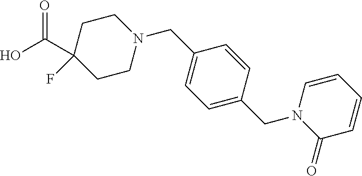 [SMILES: O=C(O)C1(F)CCN(Cc2ccc(Cn3ccccc3=O)cc2)CC1] | 4-Fluoro-1-(4-((2- oxopyridin-1(2H)- yl)methyl)benzyl)piperidine- 4-carboxylic acid | Calcd.: 345.4 Found: 345.1 |
| Q4-c | 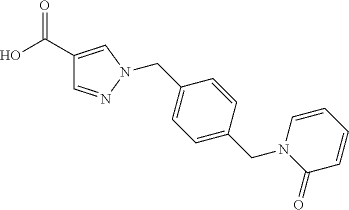 [SMILES: O=C(O)c1cnn(Cc2ccc(Cn3ccccc3=O)cc2)c1] | 1-(4-((2-Oxopyridin- 1(2H)-yl)methyl)benzyl)- 1H-pyrazole-4- carboxylic acid | Calcd.: 310.1 Found: 310.0 |
| Q4-d | 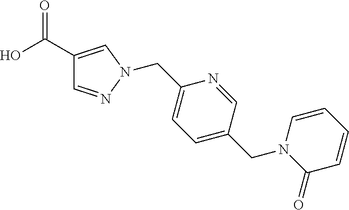 [SMILES: O=C(O)c1cnn(Cc2ccc(Cn3ccccc3=O)cn2)c1] | 1-((5-((2-Oxopyridin- 1(2H)-yl)methyl)pyridin- 2-yl)methyl)-1H- pyrazole-4-carboxylic acid | Calcd.: 311.1 Found: 311.0 |
| Q4-e | 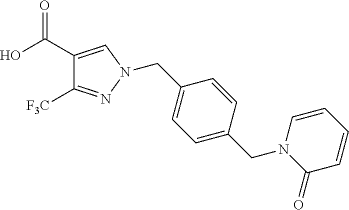 [SMILES: O=C(O)c1cn(Cc2ccc(Cn3ccccc3=O)cc2)nc1C(F)(F)F] | 1-(4-((2-Oxopyridin- 1(2H)-yl)methyl)benzyl)- 3-(trifluoromethyl)-1H- pyrazole-4-carboxylic acid | Calcd.: 378.1 Found: 378.1 |
| Q4-f | 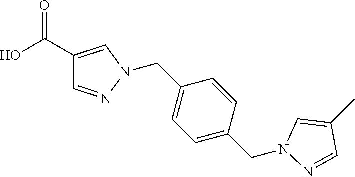 [SMILES: Cc1cnn(Cc2ccc(Cn3cc(C(=O)O)cn3)cc2)c1] | 1-(4-((4-Methyl-1H- pyrazol-2- yl)methyl)benzyl)-1H- pyrazole-4-carboxylic acid | Calcd.: 297.1 Found: 297.1 |

### Intermediate S2-a

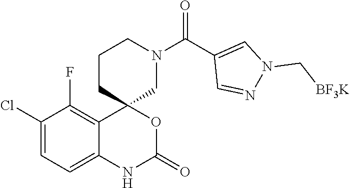

### Potassium (R)-((4-(6-chloro-5-fluoro-2-oxo-1,2-dihydrospiro[benzo[d][1.3]oxazine-4,3′-piperidin]-1′-ylcarbonyl)-1H-pyrazol-1-yl)methyl)trifluoroborate

Step 1: (R)-6-Chloro-5-fluoro-1′-(1H-pyrazole-4-carbonyl)spiro[benzo[d][1,3]oxazine-4,3′-piperidin]-2(1H)-one. HATU (743 mg, 1.95 mmol) was added to a stirred solution of Intermediate A6-d (500 mg, 1.628 mmol), 1H-pyrazole-4-carboxylic acid (192 mg, 1.71 mmol) and DIEA (0.85 ml, 4.9 mmol) were mixed in DMF (16 ml), and the resulting mixture was stirred at rt overnight. The reaction was diluted with EtOAc and washed with water. The layers were separated, and the aq. layer was extracted with EtOAc. The combined organics were washed with brine, dried (MgSO₄), filtered and concentrated under reduced pressure. The crude residue was purified by silica gel chromatography ((30% EtOH:EtOAc)/hexanes) to afford the title compound. LCMS [M+H]⁺=364.9 (calcd. 365.1).

Step 2: Potassium (R)-((4-(6-chloro-5-fluoro-2-oxo-1,2-dihydrospiro[benzo[d][1,3]oxazine-4,3′-piperidin]-1′-ylcarbonyl)-1H-pyrazol-1-yl)methyl)trifluoroborate. To a solution of (R)-6-chloro-5-fluoro-1′-(1H-pyrazole-4-carbonyl)spiro[benzo[d][1,3]oxazine-4,3′-piperidin]-2(1H)-one (30 mg, 0.082 mmol) in THE (2 mL) was added potassium (bromomethyl)trifluoroborate (17 mg, 0.082 mmol) and KHMDS (16 mg, 0.082 mmol), and the resulting mixture was heated to 60° C. After 2 h the reaction was cooled to rt, quenched by addition of MeOH, and concentrated to afford a crude residue that was purified by and purified by reverse phase HPLC (ACN/water with 0.05% TFA modifier) to the title compound. LCMS [M-39]⁺=445.1 (calcd. 445.1)¹H NMR (500 MHz, DMSO-d₆) δ 10.58 (br s, 1H), 7.76 (br s, 1H), 7.49-7.58 (m, 1H), 6.87-7.24 (m, 1H), 6.73 (br d, J=8.6 Hz, 1H), 4.17-4.84 (m, 2H), 3.67-3.84 (m, 1H), 3.15-3.32 (m, 1H), 3.02 (br s, 2H), 2.24-2.35 (m, 1H), 2.19 (br s, 1H), 1.85 (br s, 1H), 1.61 (br d, J=11.5 Hz, 1H).

### EXAMPLES

### Examples 1 and 2

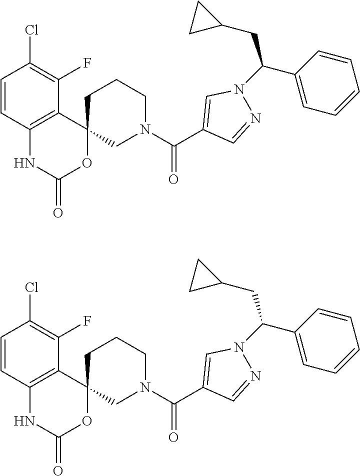 [SMILES: O=C1Nc2ccc(Cl)c(F)c2[C@@]2(CCCN(C(=O)c3cnn([C@@H](CC4CC4)c4ccccc4)c3)C2)O1 ; O=C1Nc2ccc(Cl)c(F)c2[C@@]2(CCCN(C(=O)c3cnn([C@H](CC4CC4)c4ccccc4)c3)C2)O1]

### (R)-6-Chloro-1′-(1-((S)-2-cyclopropyl-1-phenylethyl)-1H-pyrazole-4-carbonyl)-5-fluorospiro[benzo[d][1,3]oxazine-4,3′-piperidin]-2(1H)-one and (R)-6-Chloro-1′-(1-((R)-2-cyclopropyl-1-phenylethyl)-1H-pyrazole-4-carbonyl)-5-fluorospiro[benzo[d][1,3]oxazine-4,3′-piperidin]-2(1H)-one

TEA (65 μL, 0.47 mmol) was added to a stirred mixture of Intermediate C5-b (60 mg, 0.23 mmol), Intermediate A6-d (104 mg, 0.280 mmol), HATU (134 mg, 0.351 mmol) in DCM (1 mL), and the reaction was allowed to stir at rt for 2 h. The reaction was filtered, and the filtrate was concentrated to afford a crude residue that was purified by preparative reverse phase HPLC (ACN/water+0.05% TFA) to the title compound as a mixture of diastereomers. The title compounds were separated by SFC (Instrument SFC-17 Method Column DAICEL CHIRALCEL OJ-H (250 mm*30 mm, 5 um); Condition 0.1% NH₃H₂O EtOH Begin B 15%; End B 15% Gradient Time (min); 100% B). The faster eluting isomer of the title compound was obtained (Example 1): ¹H NMR (CD₃OD, 500 MHz) δ 7.90-8.42 (m, 1H), 7.58-7.87 (m, 1H), 7.12-7.50 (m, 6H), 6.73 (br d, J=12.1 Hz, 1H), 5.34-5.63 (m, 1H), 4.53-4.77 (m, 1H), 4.30 (br s, 1H), 2.94 (br s, 1H), 2.23-2.68 (m, 3H), 1.93-2.21 (m, 2H), 1.73 (br d, J=11.7 Hz, 1H), 1.22-1.42 (m, 1H), 0.55 (br s, 1H), 0.39 (br s, 2H), 0.09 (br s, 2H). LCMS [M+H]⁺=509.1 (calcd. 509.2). The slower eluting isomer of the title compound was obtained (Example 2): ¹H NMR (CD₃OD, 500 MHz) δ 7.81-8.23 (m, 1H), 7.49-7.79 (m, 1H), 7.03-7.40 (m, 6H), 6.60 (br s, 1H), 5.19-5.62 (m, 1H), 4.51 (s, 1H), 4.22 (br s, 1H), 2.85 (br s, 1H), 2.13-2.54 (m, 3H), 1.82-2.11 (m, 2H), 1.63 (br d, J=12.1 Hz, 1H), 1.06-1.32 (m, 1H), 0.11-0.55 (m, 3H), 0.00 (br s, 2H). LCMS [M+H]⁺=509.1 (calcd. 509.2).

Following procedures similar to those described above for Examples 1 and 2 and using appropriate starting materials, the following compounds were prepared:

| | | | Exact Mass | |
|---|---|---|---|---|
| Example | Structure | Name | [M + H]⁺ | Chiral Column |
| 3 | 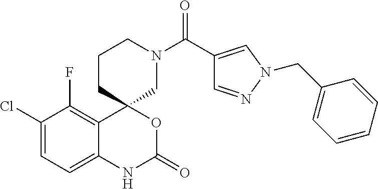 [SMILES: O=C1Nc2ccc(Cl)c(F)c2[C@@]2(CCCN(C(=O)c3cnn(Cc4ccccc4)c3)C2)O1] | (R)-1′-(1-Benzyl-1H- pyrazole-4- carbonyl)-6-chloro- 5- fluorospiro[benzo[d] [1,3]oxazine-4,3′- piperidin]-2(1H)-one | Calcd.: 454.9 Found: 455.0 | |
| 4 | 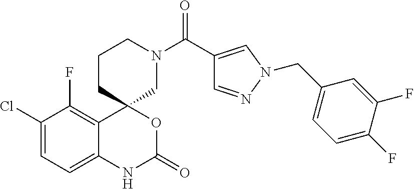 [SMILES: O=C1Nc2ccc(Cl)c(F)c2[C@@]2(CCCN(C(=O)c3cnn(Cc4ccc(F)c(F)c4)c3)C2)O1] | (R)-6-Chloro-1′-(1- (3,4-difluorobenzyl)- 1H-pyrazole-4- carbonyl)-5- fluorospiro[benzo[d] [1,3]oxazine-4,3′- piperidin]-2(1H)-one | Calcd.: 490.9 Found: 491.0 | |
| 5 | 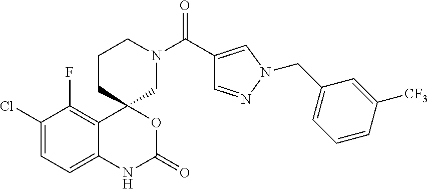 [SMILES: O=C1Nc2ccc(Cl)c(F)c2[C@@]2(CCCN(C(=O)c3cnn(Cc4cccc(C(F)(F)F)c4)c3)C2)O1] | (R)-6-Chloro-5- fluoro-1′-(1-(3- (trifluoromethyl)ben- zyl)-1H-pyrazole-4- carbonyl)spiro[benzo [d][1,3]oxazine-4,3′- piperidin]-2(1H)-one | Calcd.: 522.9 Found: 523.4 | |
| 6 | 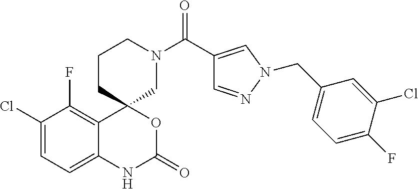 [SMILES: O=C1Nc2ccc(Cl)c(F)c2[C@@]2(CCCN(C(=O)c3cnn(Cc4ccc(F)c(Cl)c4)c3)C2)O1] | (R)-6-Chloro-1′-(1- (3-chloro-4- fluorobenzyl)-1H- pyrazole-4- carbonyl)-5- fluorospiro[benzo[d] [1,3]oxazine-4,3′- piperidin]-2(1H)-one | Calcd.: 507.3 Found: 507.3 | |
| 7 | 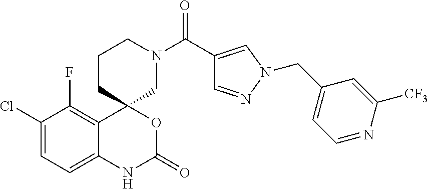 [SMILES: O=C1Nc2ccc(Cl)c(F)c2[C@@]2(CCCN(C(=O)c3cnn(Cc4ccnc(C(F)(F)F)c4)c3)C2)O1] | (R)-6-Chloro-5- fluoro-1′-(1-((2- (trifluoromethyl)pyri- din-4-yl)methyl)-1H- pyrazole-4- carbonyl)spiro[benzo [d][1,3]oxazine-4,3′- piperidin]-2(1H)-one | Calcd.: 523.9 Found: 524.0 | |
| 8 | 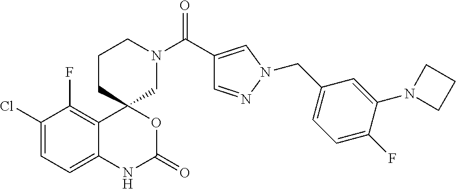 [SMILES: O=C1Nc2ccc(Cl)c(F)c2[C@@]2(CCCN(C(=O)c3cnn(Cc4ccc(F)c(N5CCC5)c4)c3)C2)O1] | (R)-1′-(1-(3- (Azetidin-1-yl)-4- fluorobenzyl)-1H- pyrazole-4- carbonyl)-6-chloro- 5- fluorospiro[benzo[d] [1,3]oxazine-4,3′- piperidin]-2(1H)-one | Calcd.: 528.0 Found: 528.1 | |
| 9 | 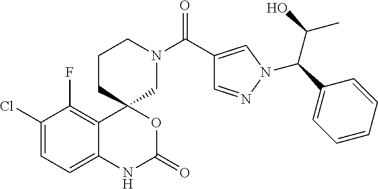 [SMILES: C[C@H](O)[C@@H](c1ccccc1)n1cc(C(=O)N2CCC[C@@]3(C2)OC(=O)Nc2ccc(Cl)c(F)c23)cn1] | (R)-6-Chloro-5- fluoro-1′-(1- ((1R,2S)-2-hydroxy- 1-phenylpropyl)-1H- pyrazole-4- carbonyl)spiro[benzo [d][1,3]oxazine-4,3′- piperidin]-2(1H)-one | Calcd.: 499.0 Found: 499.4 | |
| 10 | 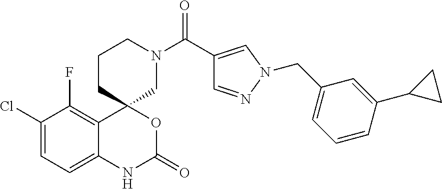 [SMILES: O=C1Nc2ccc(Cl)c(F)c2[C@@]2(CCCN(C(=O)c3cnn(Cc4cccc(C5CC5)c4)c3)C2)O1] | (R)-6-Chloro-1′-(1- (3- cyclopropylbenzyl)- 1H-pyrazole-4- carbonyl)-5- fluorospiro[benzo[d] [1,3]oxazine-4,3′- piperidin]-2(1H)-one | Calcd.: 495.0 Found: 495.0 | |
| 11 | 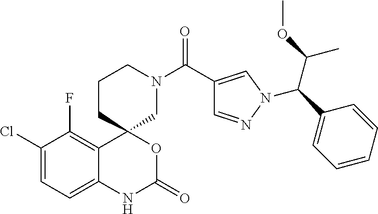 [SMILES: CO[C@@H](C)[C@@H](c1ccccc1)n1cc(C(=O)N2CCC[C@@]3(C2)OC(=O)Nc2ccc(Cl)c(F)c23)cn1] | (R)-6-Chloro-5- fluoro-1′-(1- ((1R,2S)-2-methoxy- 1-phenylpropyl)-1H- pyrazole-4- carbonyl)spiro[benzo [d][1,3]oxazine-4,3′- piepridine]-2(1H)-one | Calcd.: 513.0 Found: 513.4 | |
| 12 | 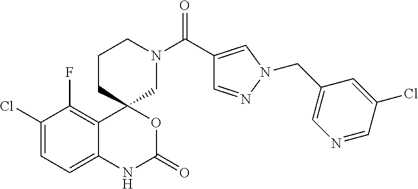 [SMILES: O=C1Nc2ccc(Cl)c(F)c2[C@@]2(CCCN(C(=O)c3cnn(Cc4cncc(Cl)c4)c3)C2)O1] | (R)-6-Chloro-1′-(1- ((5-chloropyridin-3- yl)methyl)-1H- pyrazole-4- carbonyl)-5- fluorospiro[benzo[d] [1,3]oxazine-4,3′- piperidin]-2(1H)-one | Calcd.: 490.3 Found: 490.4 | |
| 13 | 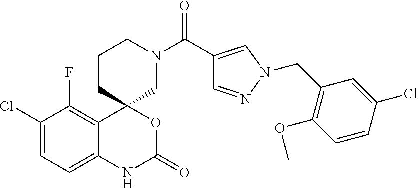 [SMILES: COc1ccc(Cl)cc1Cn1cc(C(=O)N2CCC[C@@]3(C2)OC(=O)Nc2ccc(Cl)c(F)c23)cn1] | (R)-6-Chloro-1′-(1- (5-chloro-2- methoxybenzyl)-1H- pyrazole-4- carbonyl)-5- fluorospiro[benzo[d] [1,3]oxazine-4,3′- piperidin]-2(1H)-one | Calcd.: 519.4 Found: 519.0 | |
| 14 | 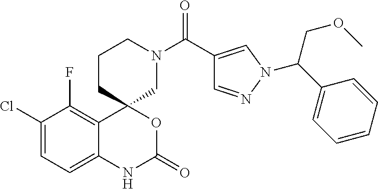 [SMILES: COCC(c1ccccc1)n1cc(C(=O)N2CCC[C@@]3(C2)OC(=O)Nc2ccc(Cl)c(F)c23)cn1] | (4R)-6-Chloro-5- fluoro-1′-(1-(2- methoxy-1- phenylethyl)-1H- pyrazole-4- carbonyl)spiro[benzo [d][1,3]oxazine-4,3′- piperidin]-2(1H)-one | Calcd.: 499.0 Found: 499.1 | |
| 15 | 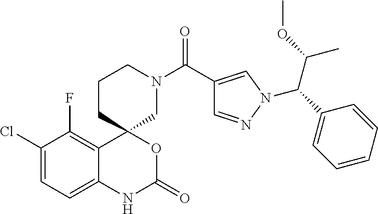 [SMILES: CO[C@H](C)[C@H](c1ccccc1)n1cc(C(=O)N2CCC[C@@]3(C2)OC(=O)Nc2ccc(Cl)c(F)c23)cn1] | (R)-6-Chloro-5- fluoro-1′-(1- ((1S,2R)-2-methoxy- 1-phenylpropyl)-1H- pyrazole-4- carbonyl)spiro[benzo [d][1,3]oxazine-4,3′- piperidin]-2(1H)-one | Calcd.: 513.0 Found: 513.4 | |
| 16 | 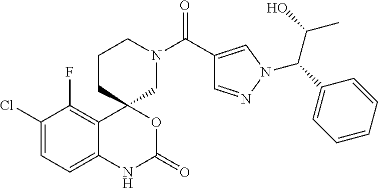 [SMILES: C[C@@H](O)[C@H](c1ccccc1)n1cc(C(=O)N2CCC[C@@]3(C2)OC(=O)Nc2ccc(Cl)c(F)c23)cn1] | (R)-6-Chloro-5- fluoro-1′-(1- ((1S,2R)-2-hydroxy- 1-phenylpropyl)-1H- pyrazole-4- carbonyl)spiro[benzo [d][1,3]oxazine-4,3′- piperidin]-2(1H)-one | Calcd.: 499.0 Found: 499.4 | |
| 17 | 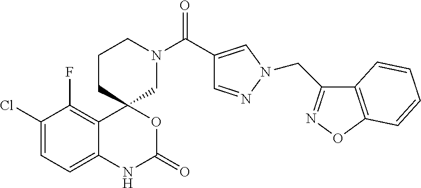 [SMILES: O=C1Nc2ccc(Cl)c(F)c2[C@@]2(CCCN(C(=O)c3cnn(Cc4noc5ccccc45)c3)C2)O1] | (R)-1′-(1- (Benzo[d]isoxazol-3- ylmethyl)-1H- pyrazole-4- carbonyl)-6-chloro- 5- fluorospiro[benzo[d] [1,3]oxazine-4,3′- piepridin]-2(1H)-one | Calcd.: 496.0 Found: 496.1 | |
| 18 | 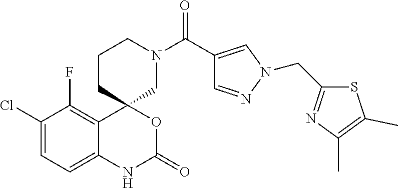 [SMILES: Cc1nc(Cn2cc(C(=O)N3CCC[C@@]4(C3)OC(=O)Nc3ccc(Cl)c(F)c34)cn2)sc1C] | (R)-6-Chloro-1′-(1- ((4,5- dimethylthiazol-2- yl)methyl)-1H- pyrazole-4- carbonyl)-5- fluorospiro[benzo[d] [1,3]oxazine-4,3′- piperidin]-2(1H)-one | Calcd.: 490.0 Found: 490.1 | |
| 19 | 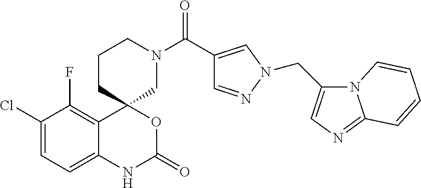 [SMILES: O=C1Nc2ccc(Cl)c(F)c2[C@@]2(CCCN(C(=O)c3cnn(Cc4cnc5ccccn45)c3)C2)O1] | (R)-6-Chloro-5- fluoro-1′-(1- (imidazo[1,2- a]pyridin-3- ylmethyl)-1H- pyrazole-4- carbonyl)spiro[benzo [d][1,3]oxazine-4,3′- piperidin]-2(1H)-one | Calcd.: 495.0 Found: 495.1 | |
| 20 | 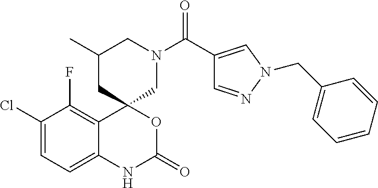 | (4R)-1′-(1-Benzyl- 1H-pyrazole-4- carbonyl)-6-chloro- 5-fluoro-5′- methylspiro[benzo[d] [1,3]oxazine-4,3′- piperidin]-2(1H)-one | Calcd.: 469.0 Found: 469.0 | |
| 21 | 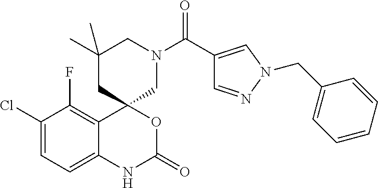 [SMILES: CC1(C)CN(C(=O)c2cnn(Cc3ccccc3)c2)C[C@]2(C1)OC(=O)Nc1ccc(Cl)c(F)c12] | (R)-1′-(1-Benzyl-1H- pyrazole-4- carbonyl)-6-chloro- 5-fluoro-5′,5′- dimethylspiro[benzo [d][1,3]oxazine-4,3′- piperidin]-2(1H)-one | Calcd.: 483.0 Found: 483.5 | |
| 22 | 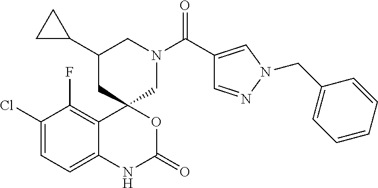 | (4R)-1′-(1-Benzyl- 1H-pyrazole-4- carbonyl)-6-chloro- 5′-cyclopropyl-5- fluorospiro[benzo[d] [1,3]oxazine-4,3′- piperidin]-2(1H)-one | Calcd.: 546.0 Found: 546.1 | |
| 23 | 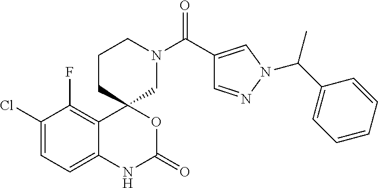 [SMILES: CC(c1ccccc1)n1cc(C(=O)N2CCC[C@@]3(C2)OC(=O)Nc2ccc(Cl)c(F)c23)cn1] | (4R)-6-Chloro-5- fluoro-1′-(1-(1- phenylethyl)-1H- pyrazole-4- carbonyl)spiro[benzo [d][1,3]oxazine-4,3′- piperidin]-2(1H)-one | Calcd: 468.9 Found: 469.4 | Whelko-1 |
| 24 | 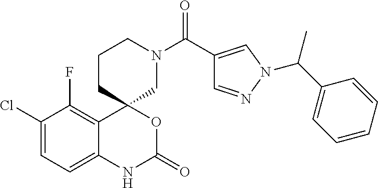 [SMILES: CC(c1ccccc1)n1cc(C(=O)N2CCC[C@@]3(C2)OC(=O)Nc2ccc(Cl)c(F)c23)cn1] | (4R)-6-Chloro-5- fluoro-1′-(1-(1- phenylethyl)-1H- pyrazole-4- carbonyl)spiro[benzo [d][1,3]oxazine-4,3′- piperidin]-2(1H)-one | Calcd.: 468.9 Found: 469.5 | Whelko-1 |
| 25 | 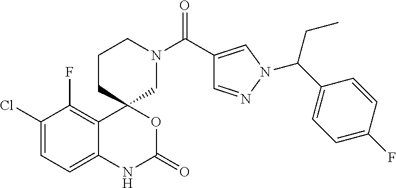 [SMILES: CCC(c1ccc(F)cc1)n1cc(C(=O)N2CCC[C@@]3(C2)OC(=O)Nc2ccc(Cl)c(F)c23)cn1] | (4R)-6-Chloro-5- fluoro-1′-(1-(1-(4- fluorophenyl)propyl)- 1H-pyrazole-4- carbonyl)spiro[benzo [d][1,3]oxazine-4,3′- piperidin]-2(1H)-one | Calcd.: 500.9 Found: 501.1 | DAICEL CHIRALPAK AD |
| 26 | 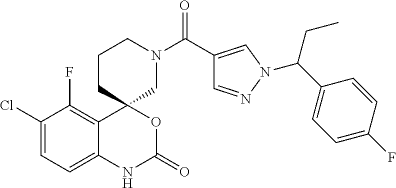 [SMILES: CCC(c1ccc(F)cc1)n1cc(C(=O)N2CCC[C@@]3(C2)OC(=O)Nc2ccc(Cl)c(F)c23)cn1] | (4R)-6-Chloro-5- fluoro-1′-(1-(1-(4- fluorophenyl)propyl)- 1H-pyrazole-4- carbonyl)spiro[benzo [d][1,3]oxazine-4,3′- piperidin]-2(1H)-one | Calcd.: 500.9 Found: 501.1 | DAICEL CHIRALPAK AD |
| 27 | 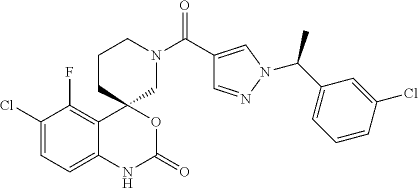 [SMILES: C[C@@H](c1cccc(Cl)c1)n1cc(C(=O)N2CCC[C@@]3(C2)OC(=O)Nc2ccc(Cl)c(F)c23)cn1] | (R)-6-Chloro-1′-(1- ((S)-1-(3- chlorophenyl)ethyl)- 1H-pyrazole-4- carbonyl)-5- fluorospiro[benzo[d] [1,3]oxazine-4,3′- piperidin]-2(1H)-one | Calcd.: 502.3 Found: 503.1 | DAICEL CHIRALPAK IC |
| 28 | 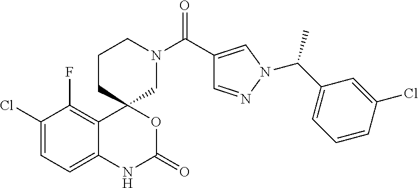 [SMILES: C[C@H](c1cccc(Cl)c1)n1cc(C(=O)N2CCC[C@@]3(C2)OC(=O)Nc2ccc(Cl)c(F)c23)cn1] | (R)-6-Chloro-1′-(1- ((R)-1-(3- chlorophenyl)ethyl)- 1H-pyrazole-4- carbonyl)-5- fluorospiro[benzo[d] [1,3]oxazine-4,3′- piperidin]-2(1H)-one | Calcd.: 503.3 Found: 503.1 | DAICEL CHIRALPAK IC |
| 29 | 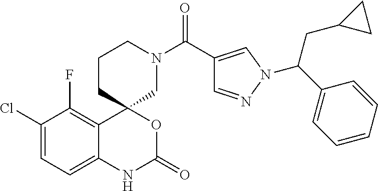 [SMILES: O=C1Nc2ccc(Cl)c(F)c2[C@@]2(CCCN(C(=O)c3cnn(C(CC4CC4)c4ccccc4)c3)C2)O1] | (4R)-6-Chloro-1′-(1- (2-cyclopropyl-1- phenylethyl)-1H- pyrazole-4- carbonyl)-5- fluorospiro[benzo[d] [1,3]oxazine-4,3′- piperidin]-2(1H)-one | Calcd.: 509.0 Found: 509.1 | YMC-Actus Pro C18 |
| 30 | 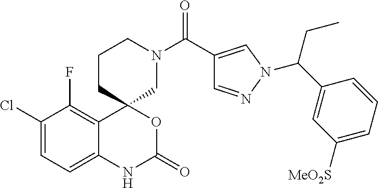 [SMILES: CCC(c1cccc(S(C)(=O)=O)c1)n1cc(C(=O)N2CCC[C@@]3(C2)OC(=O)Nc2ccc(Cl)c(F)c23)cn1] | (4R)-6-Chloro-5- fluoro-1′-(1-(1-(3- (methylsulfonyl)phe- nyl)propyl)-1H- pyrazole-4- carbonyl)spiro[benzo [d][1,3]oxazine-4,3′- piperidin]-2(1H)-one | Calcd.: 561.0 Found: 561.2 | DAICEL CHIRALPAK AD |
| 31 | 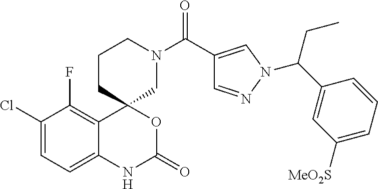 [SMILES: CCC(c1cccc(S(C)(=O)=O)c1)n1cc(C(=O)N2CCC[C@@]3(C2)OC(=O)Nc2ccc(Cl)c(F)c23)cn1] | (4R)-6-Chloro-5- fluoro-1′-(1-(1-(3- (methylsulfonyl)phe- nyl)propyl)-1H- pyrazole-4- carbonyl)spiro[benzo [d][1,3]oxazine-4,3′- piperidin]-2(1H)-one | Calcd.: 561.0 Found: 561.0 | DAICEL CHIRALPAK AD |
| 32 | 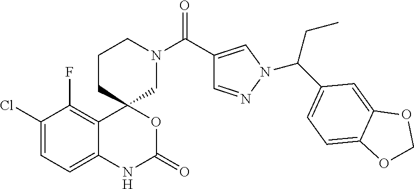 [SMILES: CCC(c1ccc2c(c1)OCO2)n1cc(C(=O)N2CCC[C@@]3(C2)OC(=O)Nc2ccc(Cl)c(F)c23)cn1] | (4R)-1′-(1-(1- (Benzo[d][1,3]odioxol- 5-yl)propyl)-1H- pyrazole-4- carbonyl)-6-chloro- 5- fluorospiro[benzo[d] [1,3]oxazine-4,3′- piperidin]-2(1H)-one | Calcd.: 527.0 Found: 527.4 | REGIS (s,s) WHELK-O1 |
| 33 | 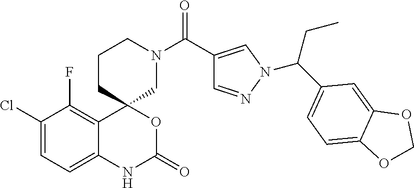 [SMILES: CCC(c1ccc2c(c1)OCO2)n1cc(C(=O)N2CCC[C@@]3(C2)OC(=O)Nc2ccc(Cl)c(F)c23)cn1] | (4R)-1′-(1-(1- (Benzo[d][1,3]dioxol- 5-yl)propyl)-1H- pyrazole-4- carbonyl)-6-chloro- 5- fluorospiro[benzo[d] [1,3]oxazine-4,3′- piepridin]-2(1H)-one | Calcd.: 527.0 Found: 527.4 | REGIS (s,s) WHELK-O1 |
| 34 | 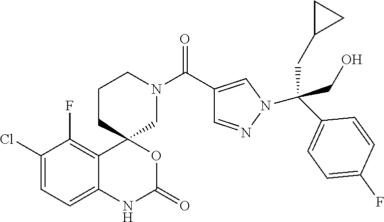 [SMILES: O=C1Nc2ccc(Cl)c(F)c2[C@@]2(CCCN(C(=O)c3cnn([C@](CO)(CC4CC4)c4ccc(F)cc4)c3)C2)O1] | (R)-6-Chloro-1′-(1- ((R)-1-cyclopropyl- 2-(4-fluorophenyl)- 3-hydroxypropan-2- yl)-1H-pyrazole-4- carbonyl)-5- fluorospiro[benzo[d] [1,3]oxazine-4,3′- piperidin]-2(1H)-one | Calcd.: 557.0 Found: 556.9 | YMC CHIRAL Amylose-C |
| 35 | 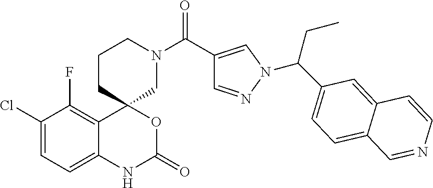 [SMILES: CCC(c1ccc2cnccc2c1)n1cc(C(=O)N2CCC[C@@]3(C2)OC(=O)Nc2ccc(Cl)c(F)c23)cn1] | (4R)-6-Chloro-5- fluoro-1′-(1-(1- (isoquinolin-6- yl)propyl)-1H- pyrazole-4- carobnyl)spiro[benzo [d][1,3]oxazine-4,3′- piperidin]-2(1H)-one | Calcd.: 534.0 Found: 534.4 | Phenomenex- Amylose-1 |
| 36 | 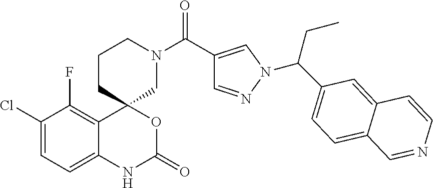 [SMILES: CCC(c1ccc2cnccc2c1)n1cc(C(=O)N2CCC[C@@]3(C2)OC(=O)Nc2ccc(Cl)c(F)c23)cn1] | (4R)-6-Chloro-5- fluoro-1′-(1-(1- (isoquinolin-6- yl)propyl)-1H- pyrazole-4- carbonyl)spiro[benzo [d][1,3]oxazine-4,3′- piperidin]-2(1H)-one | Calcd.: 534.0 Found: 534.4 | Phenomenex- Amylose-1 |
| 37 | 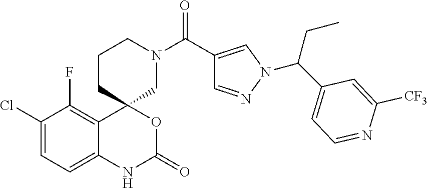 [SMILES: CCC(c1ccnc(C(F)(F)F)c1)n1cc(C(=O)N2CCC[C@@]3(C2)OC(=O)Nc2ccc(Cl)c(F)c23)cn1] | (4R)-6-Chloro-5- fluoro-1′-(1-(1-(2- (trifluoromethyl)pyri- din-4-yl)propyl)-1H- pyrazole-4- carbonyl)spiro[benzo [d][1,3]oxazine-4,3′- piperidin]-2(1H)-one | Calcd.: 551.9 Found: 551.0 | |
| 38 | 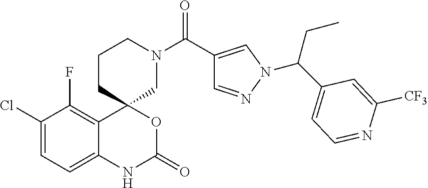 [SMILES: CCC(c1ccnc(C(F)(F)F)c1)n1cc(C(=O)N2CCC[C@@]3(C2)OC(=O)Nc2ccc(Cl)c(F)c23)cn1] | (4R)-6-Chloro-5- fluoro-1′-(1-(1-(2- (trifluoromethyl)pyri- din-4-yl)propyl)-1H- pyrazole-4- carbonyl)spiro[benzo [d][1,3]oxazine-4,3′- piperidin]-2(1H)-one | Calcd.: 551.9 Found: 551.0 | |
| 39 | 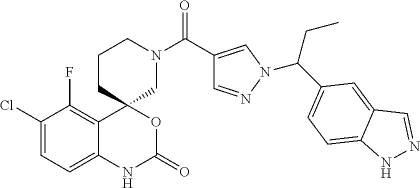 [SMILES: CCC(c1ccc2[nH]ncc2c1)n1cc(C(=O)N2CCC[C@@]3(C2)OC(=O)Nc2ccc(Cl)c(F)c23)cn1] | (4R)-1′-(1-(1-(1H- Indazol-5-yl)propyl)- 1H-pyrazole-4- carbonyl)-6-chloro- 5- fluorospiro[benzo[d] [1,3]oxazine-4,3′- piperidin]-2(1H)-one | Calcd.: 523.0 Found: 523.1 | DAICEL CHIRALCEL OD-H |
| 40 | 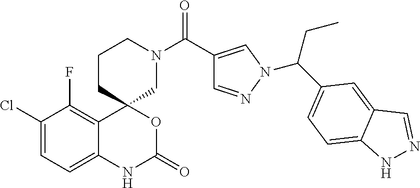 [SMILES: CCC(c1ccc2[nH]ncc2c1)n1cc(C(=O)N2CCC[C@@]3(C2)OC(=O)Nc2ccc(Cl)c(F)c23)cn1] | (4R)-1′-(1-(1-(1H- Indazol-5-yl)propyl)- 1H-pyrazole-4- carbonyl)-6-chloro- 5- fluorospiro[benzo[d] [1,3]oxazine-4,3′- piperidin]-2(1H)-one | Calcd.: 523.0 Found: 523.1 | DAICEL CHIRALCEL OD-H |
| 41 | 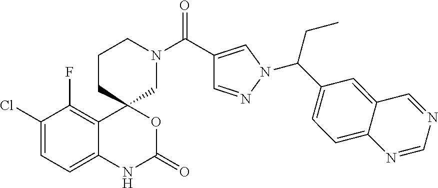 [SMILES: CCC(c1ccc2ncncc2c1)n1cc(C(=O)N2CCC[C@@]3(C2)OC(=O)Nc2ccc(Cl)c(F)c23)cn1] | (4R)-6-Chloro-5- fluoro-1′-(1-(1- (quinazolin-6- yl)propyl)-1H- pyrazole-4- carbonyl)spiro[benzo [d][1,3]oxazine-4,3′- piperidin]-2(1H)-one | Calcd.: 535.0 Found: 535.2 | Waters XSELECT C18 |
| 42 | 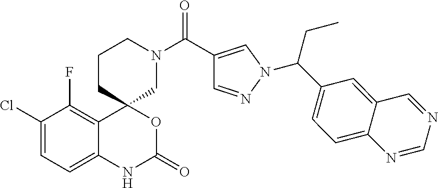 [SMILES: CCC(c1ccc2ncncc2c1)n1cc(C(=O)N2CCC[C@@]3(C2)OC(=O)Nc2ccc(Cl)c(F)c23)cn1] | (4R)-6-Chloro-5- fluoro-1′-(1-(1- (quinazolin-6- yl)propyl)-1H- pyrazole-4- carbonyl)spiro[benzo [d][1,3]oxazine-4,3′- piperidin]-2(1H)-one | Calcd.: 535.0 Found: 535.2 | Waters XSELECT C18 |
| 43 | 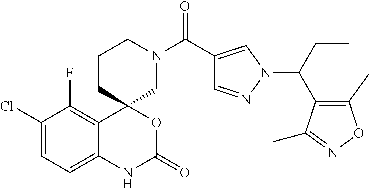 [SMILES: CCC(c1c(C)noc1C)n1cc(C(=O)N2CCC[C@@]3(C2)OC(=O)Nc2ccc(Cl)c(F)c23)cn1] | (4R)-6-Chloro-1′-(1- (1-(3,5- dimethylisoxazol-4- yl)propyl)-1H- pyrazole-4- carbonyl)-5- fluorospiro[benzo[d] [1,3]oxazine-4,3′- piperidin]-2(1H)-one | Calcd.: 501.9 Found: 502.2 | Waters XSELECT C18 |
| 44 | 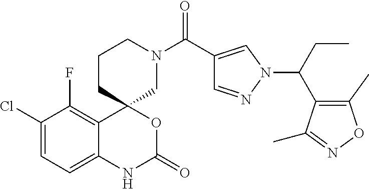 [SMILES: CCC(c1c(C)noc1C)n1cc(C(=O)N2CCC[C@@]3(C2)OC(=O)Nc2ccc(Cl)c(F)c23)cn1] | (4R)-6-Chloro-1′-(1- (1-(3,5- dimethylisoxazol-4- yl)propyl)-1H- pyrazole-4- carbonyl)-5- fluorospiro[benzo[d] [1,3]oxazine-4,3′- piperidin]-2(1H)-one | Calcd.: 501.9 Found: 502.2 | Waters XSELECT C18 |
| 45 | 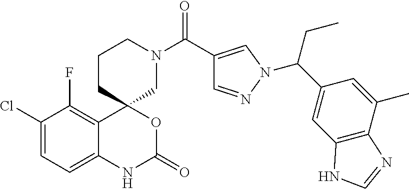 [SMILES: CCC(c1cc(C)c2nc[nH]c2c1)n1cc(C(=O)N2CCC[C@@]3(C2)OC(=O)Nc2ccc(Cl)c(F)c23)cn1] | (4R)-6-Chloro-5- fluoro-1′-(1-((R or S)-1-(4-methyl-1H- benzo[d]imidazol-6- yl)propyl)-1H- pyrazole-4- carbonyl)spiro[benzo [d][1,3]oxazine-4,3′- piperidin]-2(1H)-one | Calcd.: 529.0 Found: 529.2 | Agela DuraShell C18 |
| 46 | 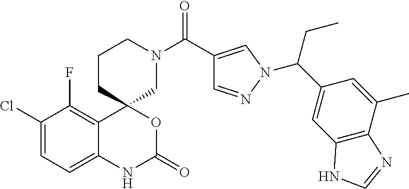 [SMILES: CCC(c1cc(C)c2nc[nH]c2c1)n1cc(C(=O)N2CCC[C@@]3(C2)OC(=O)Nc2ccc(Cl)c(F)c23)cn1] | (4R)-6-Chloro-5- fluoro-1′-(1-((S or R)-1-(4-methyl-1H- benzo[d]imidazol-6- yl)propyl)-1H- pyrazole-4- carbonyl)spiro[benzo [d][1,3]oxazine-4,3′- piperidin]-2(1H)-one | Calcd.: 537.9 Found: 538.2 | Agela DuraShell C18 |

### Example 47

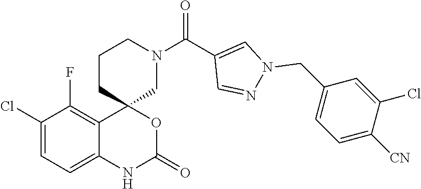 [SMILES: N#Cc1ccc(Cn2cc(C(=O)N3CCC[C@@]4(C3)OC(=O)Nc3ccc(Cl)c(F)c34)cn2)cc1Cl]

### (R)-2-Chloro-4-((4-(6-chloro-5-fluoro-2-oxo-1,2-dihydrospiro[benzo[d][1.3]oxazine-4,3′-piperidine]-1′-carbonyl)-1H-pyrazol-1-yl)methyl)benzonitrile

4-Bromo-2-chlorobenzonitrile (5.0 mg, 0.023 mmol), Cs₂CO₃ (30 mg, 0.092 mmol) and 1,1′-bis(diphenylphosphino)ferrocene-palladium (II) dichloride dichloromethane complex (3 mg, 0.003 mmol) were added to a stirred solution of Intermediate S2-a (13 mg, 0.028 mmol) in dioxane (0.3 mL) and water (0.1 mL). The resulting mixture was degassed via N₂ stream, the reaction vessel was sealed, and the reaction mixture was heated to 90° C. and allowed to stir overnight. The reaction was cooled to rt, diluted with water and extracted with EtOAc. The combined organic phases were washed with brine, dried (MgSO₄) and concentrated under reduced pressure to afford a crude residue that was purified by preparative TLC (MeOH/DCM) to give the title compound. ¹H NMR (500 MHz, CDCl₃) δ 7.89 (s, 1H), 7.71 (s, 1H), 7.66 (d, J=7.9 Hz, 1H), 7.37 (s, 1H), 7.33 (m, 1H), 7.23 (d, J=8.0 Hz, 1H), 6.61 (d, J=7.9 Hz, 1H), 5.33 (s, 2H), 4.80 (s, 1H), 4.37 (bs, 1H), 3.25 (s, 1H), 2.46 (s, 1H), 2.39 (s, 1H), 2.34 (d, J=12.0 Hz, 1H), 1.72 (m, 1H), 1.24 (s, 1H). LCMS [M+H]⁺=514.0 (calcd. 514.1).

Following procedures similar to those described above for Example 47 and using appropriate starting materials, the following compounds were prepared:

| | | | Exact Mass |
|---|---|---|---|
| Example | Structure | Name | [M + H]⁺ |
| 48 | 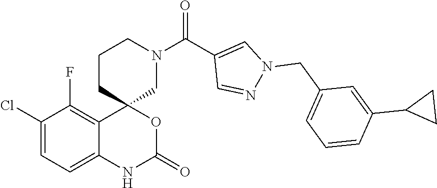 [SMILES: O=C1Nc2ccc(Cl)c(F)c2[C@@]2(CCCN(C(=O)c3cnn(Cc4cccc(C5CC5)c4)c3)C2)O1] | (R)-6-Chloro-1′-(1- (3- cyclopropylbenzyl)- 1H-pyrazole-4- carbonyl)-5- fluorospiro[benzo[d] [1,3]oxazine-4,3′- piperidin]-2(1H)- one | Calcd.: 495.0 Found: 495.0 |
| 49 | 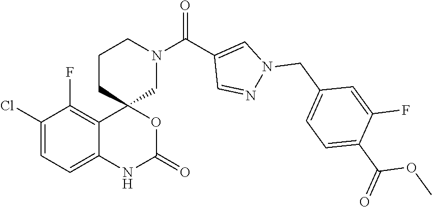 [SMILES: COC(=O)c1ccc(Cn2cc(C(=O)N3CCC[C@@]4(C3)OC(=O)Nc3ccc(Cl)c(F)c34)cn2)cc1F] | Methyl (R)-4-((4-(6- chloro-5-fluoro-2- oxo-1,2- dihydrospiro[benzo [d][1,3]oxazine-4,3′- piperidine]-1′- carbonyl)-1H- pyrazol-1- yl)methyl)-2- fluorobenzoate | Calcd.: 495.9 Found: 496.2 |
| 50 | 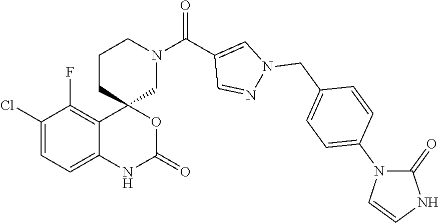 [SMILES: O=C1Nc2ccc(Cl)c(F)c2[C@@]2(CCCN(C(=O)c3cnn(Cc4ccc(-n5cc[nH]c5=O)cc4)c3)C2)O1] | (R)-6-Chloro-5- fluoro-1′-(1-(4-(2- oxo-2,3-dihydro- 1H-imidazol-1- yl)benzyl)-1H- pyrazole-4- carbonyl)spiro[benzo [d][1,3]oxazine- 4,3′-piperidin]- 2(1H)-one | Calcd.: 531.0 Found: 531.0 |
| 51 | 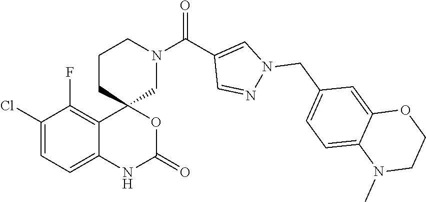 [SMILES: CN1CCOc2cc(Cn3cc(C(=O)N4CCC[C@@]5(C4)OC(=O)Nc4ccc(Cl)c(F)c45)cn3)ccc21] | (R)-6-Chloro-5- fluoro-1′-(1-((4- methyl-3,4-dihydro- 2H- benzo[b][1,4]oxazin- 7-yl)methyl)-1H- pyrazole-4- carbonyl)spiro[benzo [d][1,3]oxazine- 4,3′-piperidin]- 2(1H)-one | Calcd.: 533.3 Found: 533.1 |
| 52 | 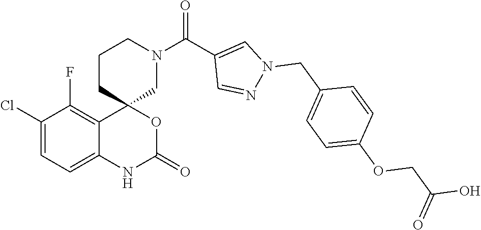 [SMILES: O=C(O)COc1ccc(Cn2cc(C(=O)N3CCC[C@@]4(C3)OC(=O)Nc3ccc(Cl)c(F)c34)cn2)cc1] | (R)-2-(4-((4-(6- Chloro-5-fluoro-2- oxo-1,2- dihydrospiro[benzo [d][1,3]oxazine-4,3′- piperidine]-1′- carbonyl)-1H- pyrazol-1- yl)methyl)phenoxy) acetic acid | Calcd.: 537.0 Found: 537.3 |
| 53 | 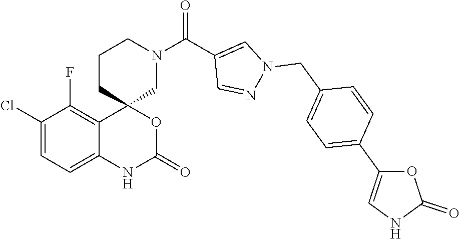 [SMILES: O=C1Nc2ccc(Cl)c(F)c2[C@@]2(CCCN(C(=O)c3cnn(Cc4ccc(-c5c[nH]c(=O)o5)cc4)c3)C2)O1] | (R)-6-Chloro-5- fluoro-1′-(1-(4-(2- oxo-2,3- dihydrooxazol-5- yl)benzyl)-1H- pyrazole-4- carbonyl)spiro[benzo [d][1,3]oxazine- 4,3′-piperidin]- 2(1H)-one | Calcd.: 526.0 Found: 526.2 |
| 54 | 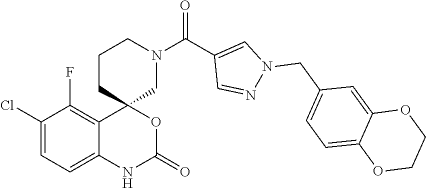 [SMILES: O=C1Nc2ccc(Cl)c(F)c2[C@@]2(CCCN(C(=O)c3cnn(Cc4ccc5c(c4)OCCO5)c3)C2)O1] | (R)-6-Chloro-1′-(1- ((2,3- dihydrobenzo[b][1,4] dioxin-6- yl)methyl)-1H- pyrazole-4- carbonyl)-5- fluorospiro[benzo[d] [1,3]oxazine-4,3′- piperidin]-2(1H)- one | Calcd.: 512.9 Found: 513.1 |
| 55 | 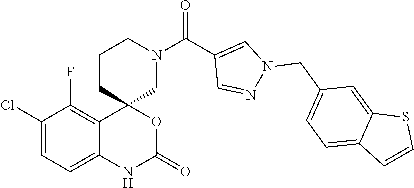 [SMILES: O=C1Nc2ccc(Cl)c(F)c2[C@@]2(CCCN(C(=O)c3cnn(Cc4ccc5ccsc5c4)c3)C2)O1] | (R)-1′-(1- (Benzo[b]thiophen- 6-ylmethyl)-1H- pyrazole-4- carbonyl)-6-chloro- 5- fluorospiro[benzo[d] [1,3]oxazine-4,3′- piperidin]-2(1H)- one | Calcd.: 511.0 Found: 511.1 |
| 56 | 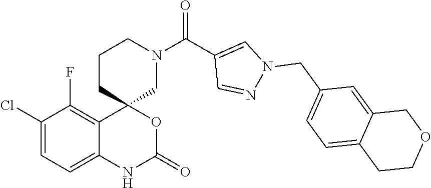 [SMILES: O=C1Nc2ccc(Cl)c(F)c2[C@@]2(CCCN(C(=O)c3cnn(Cc4ccc5c(c4)COCC5)c3)C2)O1] | (R)-6-Chloro-5- fluoro-1′-(1- (isochroman-7- ylmethyl)-1H- pyrazole-4- carbonyl)spiro[benzo [d][1,3]oxazine- 4,3′-piperidin]- 2(1H)-one | Calcd.: 511.0 Found: 511.1 |
| 57 | 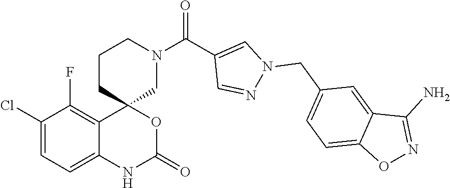 [SMILES: Nc1noc2ccc(Cn3cc(C(=O)N4CCC[C@@]5(C4)OC(=O)Nc4ccc(Cl)c(F)c45)cn3)cc12] | (R)-1′-(1-((3- Aminobenzo[d] isoxazol-5-yl)methyl)- 1H-pyrazole-4- carbonyl)-6-chloro- 5- fluorospiro[benzo[d] [1,3]oxazine-4,3′- piperidin]-2(1H)- one | Calcd.: 510.9 Found: 511.1 |
| 58 | 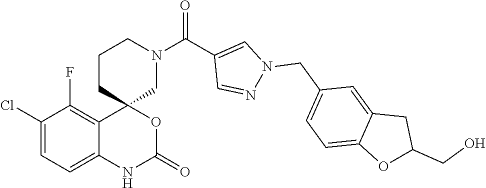 [SMILES: O=C1Nc2ccc(Cl)c(F)c2[C@@]2(CCCN(C(=O)c3cnn(Cc4ccc5c(c4)CC(CO)O5)c3)C2)O1] | (4R)-6-Chloro-5- fluoro-1′-(1-((2- (hydroxymethyl)- 2,3- dihydrobenzofuran- 5-yl)methyl)-1H- pyrazole-4- carbonyl)spiro[benzo [d][1,3]oxazine- 4,3′-piperidin]- 2(1H)-one | Calcd.: 525.0 Found: 525.2 |
| 59 | 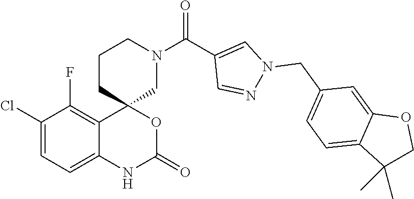 [SMILES: CC1(C)COc2cc(Cn3cc(C(=O)N4CCC[C@@]5(C4)OC(=O)Nc4ccc(Cl)c(F)c45)cn3)ccc21] | (R)-6-Chloro-1′-(1- ((3,3-dimethyl-2,3- dihydrobenzofuran- 6-yl)methyl)-1H- pyrazole-4- carbonyl)-5- fluorospiro[benzo[d] [1,3]oxazine-4,3′- piperidin]-2(1H)- one | Calcd.: 527.0 Found: 527.0 |
| 60 | 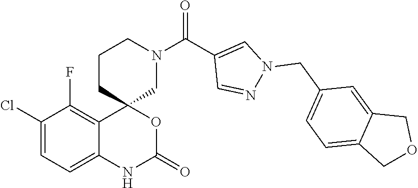 [SMILES: O=C1Nc2ccc(Cl)c(F)c2[C@@]2(CCCN(C(=O)c3cnn(Cc4ccc5c(c4)COC5)c3)C2)O1] | (R)-6-chloro-1′-(1- ((1,3- dihydroisobenzofuran- 5-yl)methyl)-1H- pyrazole-4- carbonyl)-5- fluorospiro[benzo[d] [1,3]oxazine-4,3′- piperidin]-2(1H)- one | Calcd.: 525.0 Found: 525.2 |
| 61 | 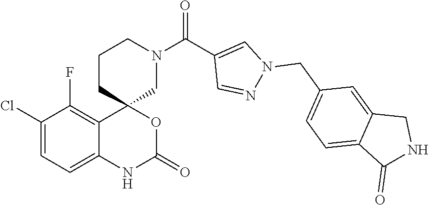 [SMILES: O=C1Nc2ccc(Cl)c(F)c2[C@@]2(CCCN(C(=O)c3cnn(Cc4ccc5c(c4)CNC5=O)c3)C2)O1] | (R)-6-chloro-5- fluoro-1′-(1-((1- oxoisoindolin-5- yl)methyl)-1H- pyrazole-4- carbonyl)spiro[benzo [d][1,3]oxazine- 4,3′-piperidin]- 2(1H)-one | Calcd.: 496.9 Found: 497.1 |
| 62 | 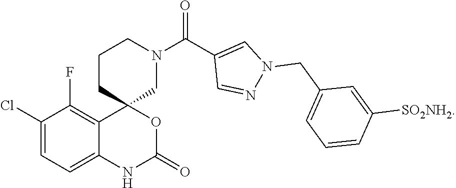 [SMILES: NS(=O)(=O)c1cccc(Cn2cc(C(=O)N3CCC[C@@]4(C3)OC(=O)Nc3ccc(Cl)c(F)c34)cn2)c1] | (R)-3-((4-(6-chloro- 5-fluoro-2-oxo-1,2- dihydrospiro[benzo [d][1,3]oxazine-4,3′- piperidine]-1′- carbonyl)-1H- pyrazol-1- yl)methyl)benzene- sulfonamide | Calcd.: 509.9 Found: 510.0 |
| 63 | 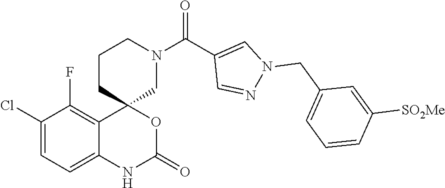 [SMILES: CS(=O)(=O)c1cccc(Cn2cc(C(=O)N3CCC[C@@]4(C3)OC(=O)Nc3ccc(Cl)c(F)c34)cn2)c1] | (R)-6-chloro-5- fluoro-1′-(1-(3- (methylsulfonyl) benzyl)-1H-pyrazole- 4-carbonyl)spiro [benzo[d][1,3]oxazine- 4,3′-piperidin]- 2(1H)-one | Calcd.: 510.0 Found: 510.1 |
| 64 | 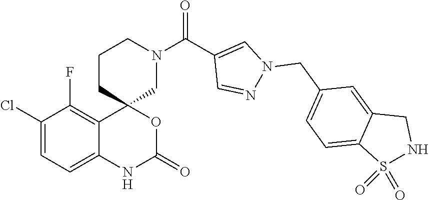 [SMILES: O=C1Nc2ccc(Cl)c(F)c2[C@@]2(CCCN(C(=O)c3cnn(Cc4ccc5c(c4)CNS5(=O)=O)c3)C2)O1] | (R)-6-chloro-1′-(1- ((1,1-dioxido-2,3- dihydrobenzo[d] isothiazol-5-yl)methyl)- 1H-pyrazole-4- carbonyl)-5- fluorospiro[benzo[d] [1,3]oxazine-4,3′- piperidin]-2(1H)- one | Calcd.: 534.0 Found: 534.0 |

### Example 65

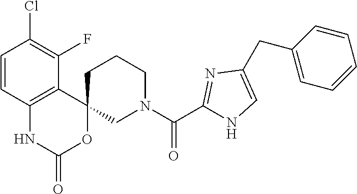 [SMILES: O=C1Nc2ccc(Cl)c(F)c2[C@@]2(CCCN(C(=O)c3nc(Cc4ccccc4)c[nH]3)C2)O1]

### (R)-1′-(4-Benzyl-1H-imidazole-2-carbonyl)-6-chloro-5-fluorospiro[benzo[d][1,3]oxazine-4,3′-piperidin]-2(1H)-one

Step 1: (R)-1′-(4-Benzyl-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one. Intermediate A6-d (105 mg, 0.284 mmol) was added to a solution of Intermediate 05-a (74 mg, 0.22 mmol) and EDC (84 mg, 0.44 mmol) in pyridine (4 mL) at 0° C. The resulting mixture was warmed to 30° C. and allowed to stir for 16 h. The reaction was diluted with water and extracted with EtOAc. The combined organic extracts were washed with 1M HCl and concentrated to afford a crude residue that was purified by preparative TLC (EtOAc/petroleum ether) to give the title compound. LCMS [M+H]⁺=585.4 (calcd. 585.2).

Step 2: (R)-1′-(4-Benzyl-1H-imidazole-2-carbonyl)-6-chloro-5-fluorospiro[benzo[d][1,3]oxazine-4,3′-piperidin]-2(1H)-one. Trifluoroacetic acid (0.5 mL) was added to a solution of (R)-1′-(4-benzyl-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one (90 mg, 0.154 mmol) in DCM (3 mL). The reaction was allowed to stir at rt for 11 h, at which time, the mixture was concentrated to afford a crude residue that was purified by reverse phase HPLC (ACN/water with 0.05% TFA modifier) to the title compound. ¹H NMR (400 MHz, CD₃OD) δ 7.11-7.59 (m, 7H), 6.63-6.88 (m, 1H), 4.47-4.70 (m, 1H), 3.87-4.33 (m, 3H), 3.47 (br d, J=12.8 Hz, 1H), 2.97-3.27 (m, 0.5H), 2.57 (br s, 0.5H), 2.32 (br s, 2H), 2.01-2.22 (m, 1H), 1.77 (br d, J=13.4 Hz, 1H). LCMS [M+H]⁺=455.2 (calcd. 455.1).

Following procedures similar to those described above for Example 65 and using appropriate starting materials, the following compounds were prepared.

| | | | Exact | |
|---|---|---|---|---|
| | | | Mass | Chiral |
| Example | Structure | Name | [M + H]⁺ | Column |
| 66 | 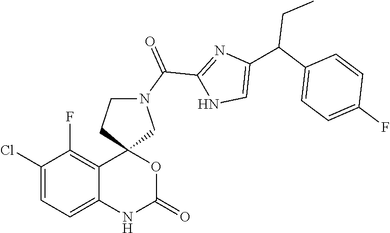 [SMILES: CCC(c1ccc(F)cc1)c1c[nH]c(C(=O)N2CC[C@@]3(C2)OC(=O)Nc2ccc(Cl)c(F)c23)n1] | (4R)-6-Chloro-5-fluoro-1′-(4-((S or R)-1-(4-fluorophenyl)propyl)-1H- imidazole-2- carbonyl)spiro[benzo[d][1,3]oxazine- 4,3′-pyrrolidin]-2(1H)-one | Calcd.: 487.1 Found: 487.2 | Cellulose- 2 |
| 67 | 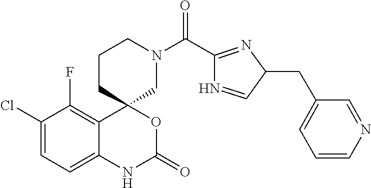 [SMILES: O=C1Nc2ccc(Cl)c(F)c2[C@@]2(CCCN(C(=O)C3=NC(Cc4cccnc4)C=N3)C2)O1] | (R)-6-Chloro-5-fluoro-1-(4′-(pyridin- 3-ylmethyl)-1H-imidazole-2- carbonyl)spiro[benzo[d][1,3]oxazine- 4,3′-piperidin]-2(1H)-one | Calcd.: 456.1 Found: 456.2 | |
| 68 | 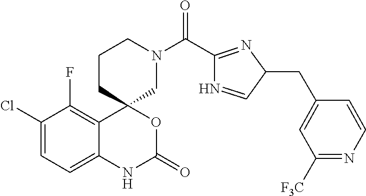 [SMILES: O=C1Nc2ccc(Cl)c(F)c2[C@@]2(CCCN(C(=O)C3=NC(Cc4ccnc(C(F)(F)F)c4)C=N3)C2)O1] | (R)-6-Chloro-5-fluoro-1′-(4-((5- (trifluoromethyl)pyridin-3- yl)methyl)-1H-imidazole-2- carbonyl)spiro[benzo[d][1,3]oxazine- 4,3′-piperidin]-2(1H)-one | Calcd.: 524.9 Found: 524.9 | |
| 69 | 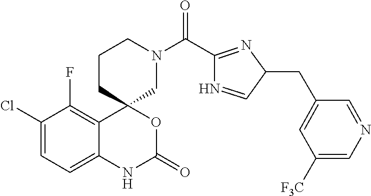 [SMILES: O=C1Nc2ccc(Cl)c(F)c2[C@@]2(CCCN(C(=O)C3=NC(Cc4cncc(C(F)(F)F)c4)C=N3)C2)O1] | (R)-6-Chloro-5-fluoro-1′-(4-((2- (trifluoromethyl)pyridin-4- yl)methyl)-1H-imidazole-2- carbonyl)spiro[benzo[d][1,3]oxazine- 4,3′-piperidin]-2(1H)-one | Calcd.: 524.9 Found: 524.0 | |
| 70 | 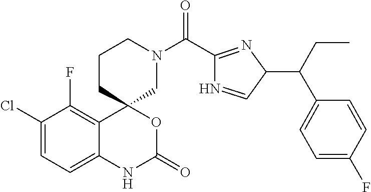 [SMILES: CCC(c1ccc(F)cc1)C1C=NC(C(=O)N2CCC[C@@]3(C2)OC(=O)Nc2ccc(Cl)c(F)c23)=N1] | (4R)-6-Chloro-5-fluoro-1′-(4-(1-(4- fluorophenyl)propyl)-1H-imidazole- 2- carbonyl)spiro[benzo[d][1,3]oxazine- 4,3′-piperidin]-2(1H)-one | Calcd.: 501.1 Found: 501.3 | |
| 71 | 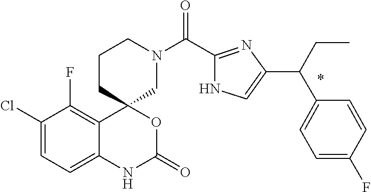 [SMILES: CCC(c1ccc(F)cc1)c1c[nH]c(C(=O)N2CCC[C@@]3(C2)OC(=O)Nc2ccc(Cl)c(F)c23)n1] | (R)-6-Chloro-5-fluoro-1′-(4-((R or S)-1-(4-fluorophenyl)propyl)-1H- imidazole-2- carbonyl)spiro[benzo[d][1,3]oxazine- 4,3′-piperidin]-2(1H)-one | Calcd.: 501.1 Found: 500.9 | OJ-H; peak A |
| 72 | 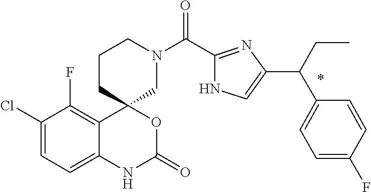 [SMILES: CCC(c1ccc(F)cc1)c1c[nH]c(C(=O)N2CCC[C@@]3(C2)OC(=O)Nc2ccc(Cl)c(F)c23)n1] | (R)-6-Chloro-5-fluoro-1′-(4-((S or R)-1-(4-fluorophenyl)propyl)-1H- imidazole-2- carbonyl)spiro[benzo[d][1,3]oxazine- 4,3′-piperidin]-2(1H)-one | Calcd.: 501.1 Found: 500.9 | OJ-H; peak B |
| 73 | 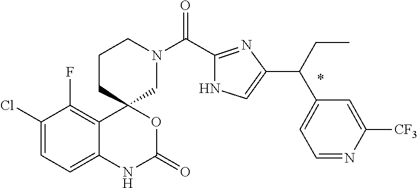 [SMILES: CCC(c1ccnc(C(F)(F)F)c1)c1c[nH]c(C(=O)N2CCC[C@@]3(C2)OC(=O)Nc2ccc(Cl)c(F)c23)n1] | (R)-6-Chloro-5-fluoro-1′-(4-((R or S)-1-(2-(trifluoromethyl)pyridin-4- yl)propyl)-1H-imidazole-2- carbonyl)spiro[benzo[d][1,3]oxazine- 4,3′-piperidin]-2(1H)-one | Calcd.: 552.1 Found: 552.2 | OJ-H; peak A |
| 74 | 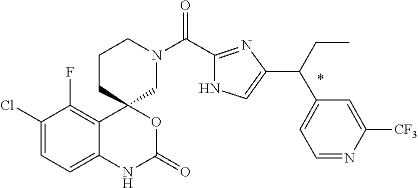 [SMILES: CCC(c1ccnc(C(F)(F)F)c1)c1c[nH]c(C(=O)N2CCC[C@@]3(C2)OC(=O)Nc2ccc(Cl)c(F)c23)n1] | (R)-6-Chloro-5-fluoro-1′-(4-((S or R)-1-(2-(trifluoromethyl)pyridin-4- yl)propyl)-1H-imidazole-2- carbonyl)spiro[benzo[d][1,3]oxazine- 4,3′-piperidin]-2(1H)-one | Calcd.: 552.1 Found: 552.2 | OJ-H; peak B |

### Examples 75

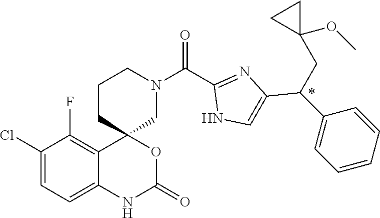 [SMILES: COC1(CC(c2ccccc2)c2c[nH]c(C(=O)N3CCC[C@@]4(C3)OC(=O)Nc3ccc(Cl)c(F)c34)n2)CC1]

### (R)-6-Chloro-5-fluoro-1′-(4-((R or S)-2-(1-methoxycyclopropyl)-1-phenylethyl)-1H-imidazole-2-carbonyl)spiro[benzo[d][1,3]oxazine-4,3′-piperidin]-2(1H)-one

Step 1: (3′R)-6-Chloro-5-fluoro-1′-(4-(hydroxy(phenyl)methyl)-1-((2-(trimethylsilyl)ethoxy)methyl)-1H-imidazole-2-carbonyl)spiro[benzo[d][1,3]oxazine-4,3′-piperidin]-2(1H)-one. To a stirred solution of (R)-2-(6-chloro-5-fluoro-2-oxo-1,2-dihydrospiro[benzo[d][1,3]oxazine-4,3′-piperidin]-1′-ylcarbonyl)-1-((2-(trimethylsilyl)ethoxy)methyl)-1H-imidazole-4-carbaldehyde Intermediate H (580 mg, 1.11 mmol) in THF (8.0 mL) was added phenylmagnesium bromide (0.74 mL of a 3.0 M THF solution. 2.218 mmol) at −15° C. The reaction was warmed to 0° C. and allowed to stir for 2 h. The reaction was quenched with satd. aq. NH₄Cl and extracted with EtOAc. The combined organic extracts were washed with brine, dried (Na₂SO₄), and concentrated to afford a crude residue that was purified by silica gel chromatography (EtOAc/petroleum ether) to give the title compound. LCMS [M+H]⁺=601.3 (calcd. 601.2).

Step 2: (R)-1′-(4-Benzoyl-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one. Manganese(IV) oxide (376 mg, 4.33 mmol) was added to a stirred solution of (3′R)-6-chloro-5-fluoro-1′-(4-(hydroxy(phenyl)methyl)-1-((2-(trimethylsilyl)ethoxy)methyl)-1H-imidazole-2-carbonyl)spiro[benzo[d][1,3]oxazine-4,3′-piperidin]-2(1H)-one (520 mg, 0.865 mmol) in DCM (3.0 mL), and the resulting mixture was allowed to stir at rt for 12 h. The reaction mixture was filtered and concentrated to afford a crude residue that was purified by silica gel chromatography (EtOAc/petroleum ether) to give the title compound. LCMS [M+H]⁺=599.1 (calcd. 599.2).

Step 3: (R,Z)-6-Chloro-5-fluoro-1′-(4-(2-(1-methoxycyclopropyl)-1-phenylvinyl)-1-((2-(trimethylsilyl)ethoxy)methyl)-1H-imidazole-2-carbonyl)spiro[benzo[d][1,3]oxazine-4,3′-piperidin]-2(1H)-one. LHMDS (1.07 mL, 1.07 mmol, 1.0 M THF solution) was added to a solution of (R)-1′-(4-benzoyl-1-((2-(trimethylsilyl)ethoxy)methyl)-1H-imidazole-2-carbonyl)-6-chloro-5-fluorospiro[benzo[d][1,3]oxazine-4,3′-piperidin]-2(1H)-one (160 mg, 0.267 mmol) and 2-(((1-methoxycyclopropyl)methyl)sulfonyl)benzo[d]thiazole (91 mg, 0.320 mmol) in THF (4 mL) in a glove box, and the resulting mixture was sealed and allowed to stir at rt for 16 h. The reaction was quenched by addition of sat. aq. NH₄Cl and water, and the resulting mixture was extracted with EtOAc. The combined organic extracts were concentrated to afford a crude residue that was purified by preparative TLC (EtOAc/petroleum ether) to give the title compound. LCMS [M+H]⁺=667.1 (calcd. 667.2).

Step 4: (4R)-6-Chloro-5-fluoro-1′-(4-(2-(1-methoxycyclopropyl)-1-phenylethyl)-1-((2-(trimethylsilyl)ethoxy)methyl)-1H-imidazole-2-carbonyl)spiro[benzo[d][1.3]oxazine-4,3′-piperidin]-2(1H)-one. Raney Ni (0.4 mg, 0.007 mmol) was added to a stirred solution of (R,Z)-6-chloro-5-fluoro-1′-(4-(2-(1-methoxycyclopropyl)-1-phenylvinyl)-1-((2-(trimethylsilyl)ethoxy)methyl)-1H-imidazole-2-carbonyl)spiro[benzo[d][1,3]oxazine-4,3′-piperidin]-2(1H)-one (10 mg, 0.015 mmol) in THF (4 mL). The resulting mixture was allowed to stir at rt under H₂ (balloon) for 16 h. The reaction mixture was filtered and concentrated to give the crude title compound that was carried on without purification. LCMS [M+H]⁺=635.2 (calcd. 635.3).

Step 5: (3′R)-5-Fluoro-1′-(4-(2-(1-methoxycyclopropyl)-1-phenylethyl)-1-((2-(trimethylsilyl)ethoxy)methyl)-1H-imidazole-2-carbonyl)spiro[benzo[d][1,3]oxazine-4,3′-piperidin]-2(1H)-one. Trifluoroacetic acid (0.3 mL) was added to a solution of (4R)-6-chloro-5-fluoro-1′-(4-(2-(1-methoxycyclopropyl)-1-phenylethyl)-1-((2-(trimethylsilyl)ethoxy)methyl)-1H-imidazole-2-carbonyl)spiro[benzo[d][1,3]oxazine-4,3′-piperidin]-2(1H)-one (10 mg, 0.015 mmol) in DCM (3 mL) at 0° C. The resulting mixture was warmed to rt and allowed to stir for 10 h. The reaction was concentrated and purified by and purified by reverse phase HPLC (ACN/water with 0.05% TFA modifier) to give the title compound as a stereochemical mixture. LCMS [M+H]⁺=539.2 (calcd. 539.2).

The title compounds were resolved by SFC (Method Column DAICEL CHIRALCEL OJ-H (250 mm*30 mm, 5 um); Mobile Phase A: water (0.1% NH₃H₂O), Mobile Phase B: EtOH; Gradient: 25% B to 25% B). The faster eluting isomer of the title compound was obtained (Example 75): ¹H NMR (400 MHz, CD₃OD) δ 7.40-7.51 (m, 1H), 7.14-7.40 (m, 3H), 7.05 (br s, 1H), 6.83-7.00 (m, 1H), 6.75 (br t, J=9.3 Hz, 1H), 6.20 (br d, J=13.7 Hz, 1H), 4.51-4.78 (m, 1H), 4.12-4.44 (m, 1H), 3.60-3.90 (m, 1H), 3.12-3.30 (m, 3H), 2.94 (br t, J=12.7 Hz, 1H), 1.89-2.80 (m, 6H), 1.55-1.79 (m, 1H), 0.90 (br t, J=6.6 Hz. 1H), 0.35-0.66 (m. 1H), −0.09-0.31 (m, 1H). LCMS [M+H]⁺=539.2 (calcd. 539.2).

### Example 76

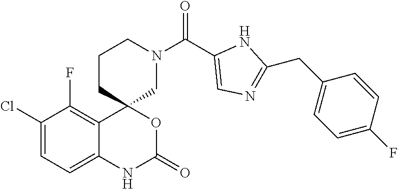 [SMILES: O=C1Nc2ccc(Cl)c(F)c2[C@@]2(CCCN(C(=O)c3cnc(Cc4ccc(F)cc4)[nH]3)C2)O1]

(R)-6-Chloro-5-fluoro-1′-(2-(4-fluorobenzyl)-1H-imidazole-5-carbonyl)spiro[benzo[d][1,3]oxazine-4,3′-piperidin]-2(1H)-one. The title compound was prepared following procedures similar to those described in Example 1. LCMS [M+H]⁺=473.0 (calcd. 473.1). ¹H NMR (600 MHz, CDCl₃) δ 1H NMR (600 MHz, CDCl₃) δ 9.85-9.42 (br m, 1H), 8.18 (s, 1H), 7.43 (s, 1H), 7.29 (m, 3H), 7.15 (m, 1H), 6.89 (m, 1H), 6.76-6.54 (m, 1H), 5.06 (br m, 1H), 4.93-4.69 (m, 1H), 4.01 (br m, 1H), 2.65-2.42 (m, 1H), 2.41-2.10 (m, 2H), 1.80-1.61 (m, 1H), 1.40-1.21 (m, 2H).

### Examples 77 and 78

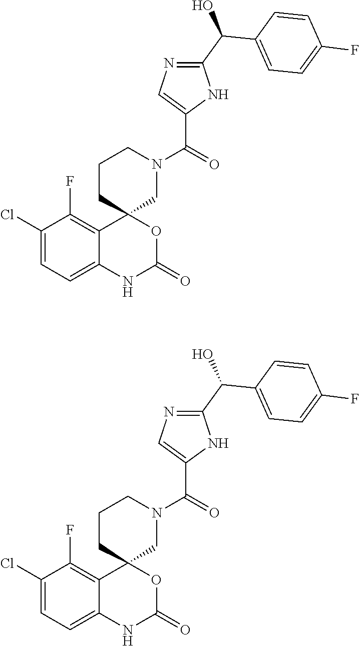 [SMILES: O=C1Nc2ccc(Cl)c(F)c2[C@@]2(CCCN(C(=O)c3cnc([C@@H](O)c4ccc(F)cc4)[nH]3)C2)O1 ; O=C1Nc2ccc(Cl)c(F)c2[C@@]2(CCCN(C(=O)c3cnc([C@H](O)c4ccc(F)cc4)[nH]3)C2)O1]

### (R)-6-Chloro-5-fluoro-1′-(2-((S)-(4-fluorophenyl)(hydroxy)methyl)-1H-imidazole-5-carbonyl)spiro[benzo[d][1,3]oxazine-4,3′-piperidin]-2(11M-one and (R)-6-Chloro-5-fluoro-1′-(2-((R)-(4-fluorophenyl)(hydroxy)methyl)-1H-imidazole-5-carbonyl)spiro[benzo[d][1.3]oxazine-4,3′-piperidin]-2(1H)-one

Step 1: (R)-6-Chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one. The title compound was prepared following procedures similar to those described in Example 1. LCMS [M+H]⁺=617.2 (calcd. 617.2).

Step 2: (3′R)-6-Chloro-5-fluoro-1′-(2-((4-fluorophenyl)(hydroxy)methyl)-1-((2-(trimethylsilyl)ethoxy)methyl)-1H-imidazole-5-carbonyl)spiro[benzo[d][1,3]oxazine-4,3′-piperidin]-2(1H)-one. Sodium borohydride (61 mg, 1.6 mmol) was added to a stirred mixture of (R)-6-chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one (1.00 g, 1.62 mmol) in THF (10 mL) at 0° C., and the resulting mixture continued stirring at 0° C. for 12 h. The reaction was concentrated, and the resulting crude residue was purified by silica gel chromatography (EtOAc/petroleum ether) to afford the title compound. LCMS [M+H]⁺=619.2 (calcd. 619.2).

Step 3: (3′R)-6-Chloro-5-fluoro-1′-(2-((4-fluorophenyl)(hydroxy)methyl)-1H-imidazole-5-carbonyl)spiro[benzo[d][1,3]oxazine-4,3′-piperidin]-2(1H)-one. The title compound was prepared as a stereochemical mixture following procedures similar to those described above in Examples 75, Step 5.

The title compounds were resolved by SFC (Column: DAICEL CHIRALPAK AD (250 mm*30 mm, 10 um); Mobile Phase A: CO₂, Mobile Phase B: EtOH (0.1% NH₃H₂O); Flow rate: 60 mL/min; Gradient 45% B to 45% B). The faster eluting isomer of the title compound was obtained (Example 77): ¹H NMR (400 MHz, CD₃OD) δ 8.30-8.60 (m, 2H), 8.18 (br s, 1H), 7.93 (s, 1H), 7.66 (br d, J=8.3 Hz, 1H), 7.60 (br s, 1H), 7.44-7.44 (m, 1H), 7.41 (br d, J=8.3 Hz, 1H), 5.26 (s, 2H), 4.47-4.65 (m, 2H), 4.30-4.45 (m, 1H), 4.02-4.22 (m, 1H), 3.62-3.98 (m, 3H), 3.34-3.62 (m, 4H), 3.04-3.28 (m, 1H), 2.91 (br d, J=7.6 Hz, 2H), 2.32-2.52 (m, 1H), 2.19-2.30 (m, 2H), 1.82-2.18 (m, 5H), 1.34 (br d, J=6.8 Hz, 2H), 1.19-1.31 (m, 1H). LCMS [M+H]⁺=489.1 (calcd. 489.1). The slower eluting isomer of the title compound was obtained (Example 78): ¹H NMR (400 MHz, CD₃OD) δ 8.30-8.60 (m, 2H), 8.18 (br s, 1H), 7.93 (s, 1H), 7.66 (br d, J=8.3 Hz, 1H), 7.60 (br s, 1H), 7.44-7.44 (m, 1H), 7.41 (br d, J=8.3 Hz, 1H), 5.26 (s, 2H), 4.47-4.65 (m, 2H), 4.30-4.45 (m, 1H), 4.02-4.22 (m, 1H), 3.62-3.98 (m, 3H), 3.34-3.62 (m, 4H), 3.04-3.28 (m, 1H), 2.91 (br d, J=7.6 Hz, 2H), 2.32-2.52 (m, 1H), 2.19-2.30 (m, 2H), 1.82-2.18 (m, 5H), 1.34 (br d, J=6.8 Hz, 2H), 1.19-1.31 (m, 1H). LCMS [M+H]⁺=489.1 (calcd. 489.1).

### Examples 79 and 80

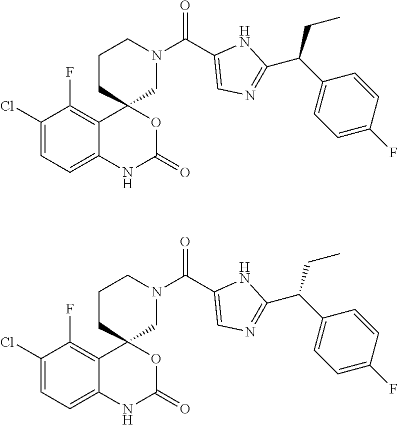 [SMILES: CC[C@@H](c1ccc(F)cc1)c1ncc(C(=O)N2CCC[C@@]3(C2)OC(=O)Nc2ccc(Cl)c(F)c23)[nH]1 ; CC[C@H](c1ccc(F)cc1)c1ncc(C(=O)N2CCC[C@@]3(C2)OC(=O)Nc2ccc(Cl)c(F)c23)[nH]1]

### (R)-6-Chloro-5-fluoro-1′-(2-((S)-1-(4-fluorophenyl)propyl)-1H-imidazole-5-carbonyl)spiro[benzo[d][1,3]oxazine-4,3′-piperidin]-2(1H)-one and (R)-6-Chloro-5-fluoro-1′-(2-((R)-1-(4-fluorophenyl)propyl)-1H-imidazole-5-carbonyl)spiro[benzo[d][1,3]oxazine-4,3′-piperidin]-2(1H)-one

Step 1: (R)-6-Chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one. The title compound was prepared following procedures similar to those described in Example 1. [M+H]⁺, 617.2, (calcd. 617.2).

Step 2: (3′R)-6-Chloro-5-fluoro-1′-(2-(1-(4-fluorophenyl)-1-hydroxypropyl)-1-((2-(trimethylsilyl)ethoxy)methyl)-1H-imidazole-5-carbonyl)spiro[benzo[d][1,3]oxazine-4,3′-piperidin]-2(1H)-one. To a solution of (R)-6-chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one (500 mg, 0.810 mmol) in THF (6 mL) was added ethylmagnesium bromide (0.324 mL, 0.972 mmol, 3 M THF solution), and the resulting mixture was allowed to stir at rt. After 1 h, the reaction was quenched with water and extracted with EtOAc. The combined organic layers were concentrated to afford a crude residue that was purified by silica gel chromatography (EtOAc/petroleum ether) to yield the title compound. LCMS [M+H]⁺=647.2, (calcd. 647.2).

Step 3: (R,E)-6-Chloro-5-fluoro-1′-(2-(1-(4-fluorophenyl)prop-1-en-1-yl)-1H-imidazole-5-carbonyl)spiro[benzo[d][1,3]oxazine-4,3′-piperidin]-2(1H)-one. BF₃·OEt₂ (0.14 mL, 1.1 mmol) was added to a stirred solution of (3′R)-6-chloro-5-fluoro-1′-(2-(1-(4-fluorophenyl)-1-hydroxypropyl)-1-((2-(trimethylsilyl)ethoxy)methyl)-1H-imidazole-5-carbonyl)spiro[benzo[d][1,3]oxazine-4,3′-piperidin]-2(1H)-one (120 mg, 0.185 mmol) and triethylsilane (0.15 mL, 0.93 mmol) in CHCl₃ (3 mL) at rt, and the resulting mixture was heated to 50° C. for 12 h. The reaction was cooled to rt, diluted with water and extracted with EtOAc. The combined organic layers were concentrated to afford the crude title compound that was carried on without further purification. LCMS [M+H]⁺=499.1, (calcd. 499.1).

Step 4: (3′R)-6-Chloro-5-fluoro-1′-(2-(1-(4-fluorophenyl)propyl)-1H-imidazole-5-carbonyl)spiro [benzo[d][1,3]oxazine-4,3′-piperidin]-2(1H)-one. Raney Ni (0.94 mg, 0.016 mmol) was added to a stirred solution of (R,E)-6-chloro-5-fluoro-1′-(2-(1-(4-fluorophenyl)prop-1-en-1-yl)-1H-imidazole-5-carbonyl)spiro[benzo[d][1,3]oxazine-4,3′-piperidin]-2(1H)-one (80 mg, 0.16 mmol) in THF (5 mL) at rt. The resulting mixture was allowed to stir at rt for 1 h, at which time, the reaction was filtered through a pad of Celite® and concentrated to afford a crude residue that was purified by and purified by reverse phase HPLC (ACN/water with 0.05% TFA modifier) to give the title compound as a stereochemical mixture. LCMS [M+H]^{m}=501.2 (calcd. 501.14).

The title compounds were resolved by SFC (Column: DAICEL CHIRALPAK AD (250 mm*30 mm, 10 um); Mobile Phase A: CO₂, Mobile Phase B: EtOH (0.1% NH₃H₂O); Flow rate: 60 mL/min; Gradient 60% B to 60% B). The faster eluting isomer of the title compound was obtained (Example 79): ¹H NMR (400 MHz, CD₃OD) δ 7.34-7.63 (m, 4H), 6.98 (br s, 2H), 6.71 (br s, 1H), 5.79 (br s, 1H), 5.50 (br s, 0.5H), 4.70 (br s. 0.5H), 3.75 (br s, 0.5H), 2.93 (br s, 0.5H), 2.41-2.59 (m, 1H), 2.27 (br d, J=13.2 Hz, 1H), 1.91-2.37 (m, 1H), 1.73 (br s, 1H), 1.20-1.41 (m, 1H). LCMS [M+H]⁺=501.2 (calcd. 501.1). The slower eluting isomer of the title compound was obtained (Example 80): ¹H NMR (400 MHz, CD₃OD) δ 7.66-8.04 (m, 1H), 7.22-7.57 (m, 3H), 7.10 (br s, 2H), 6.70 (br s, 1H), 4.63 (br s, 0.5H), 4.50 (br d, J=8.1 Hz, 0.5H), 4.26 (br s, 0.5H), 4.07 (br s, 1H), 3.34-3.56 (m, 1H), 3.00 (br d, J=18.1 Hz, 0.5H), 2.02-2.63 (m, 5H), 1.74 (br d, J=12.5 Hz, 1H), 0.93 (br s, 3H). LCMS [M+H]⁺=501.2 (calcd. 501.1).

### Example 81

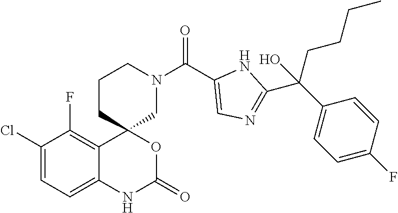 [SMILES: CCCCC(O)(c1ccc(F)cc1)c1ncc(C(=O)N2CCC[C@@]3(C2)OC(=O)Nc2ccc(Cl)c(F)c23)[nH]1]

### (3′R)-6-Chloro-5-fluoro-1′-(2-(1-(4-fluorophenyl)-1-hydroxypentyl)-1H-imidazole-5-carbonyl)spiro[benzo[d][1.3]oxazine-4,3′-piperidin]-2(1H)-one

Step 1: (3′R)-6-Chloro-5-fluoro-1′-(2-(1-(4-fluorophenyl)-1-hydroxypentyl)-1-((2-(trimethylsilyl)ethoxy)methyl)-1H-imidazole-5-carbonyl)spiro[benzo[d][1,3]oxazine-4,3′-piperidin]-2(1H)-one. n-Butyllithium (0.016 mL, 0.039 mmol, 2.5 M hexanes solution) was added to a stirred solution of (R)-6-chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one (11 μL, 0.032 mmol) in THF (2 mL) at −78° C. After 10 min, the reaction was warmed to rt and quenched with satd. aq. NH₄Cl. The mixture was extracted with EtOAc, and the combined organics were concentrated to afford the crude title compound that was carried on without further purification. LCMS [M+H]⁺=675.3 (calcd. 675.3).

Step 2: (3′R)-6-Chloro-5-fluoro-1′-(2-(1-(4-fluorophenyl)-1-hydroxypentyl)-1H-imidazole-5-carbonyl)spiro[benzo[d][1,3]oxazine-4,3′-piperidin]-2(1H)-one. To a solution of (3′R)-6-chloro-5-fluoro-1′-(2-(1-(4-fluorophenyl)-1-hydroxypentyl)-1-((2-(trimethylsilyl)ethoxy)methyl)-1H-imidazole-5-carbonyl)spiro[benzo[d][1,3]oxazine-4,3′-piperidin]-2(1H)-one (21 mg, 0.031 mmol) in DCM (2 mL) was added TFA (0.2 mL), and the resulting mixture was allowed to stir at rt. After 12 h, the reaction was concentrated to afford a crude residue that was purified by and purified by reverse phase HPLC (ACN/water with 0.05% TFA modifier) to give the title compound. ¹H NMR (400 MHz, CD₃OD) δ 7.67-8.04 (m, 1H), 7.35-8.00 (m, 3H), 7.12 (br dd, J=17.4, 8.6 Hz, 2H), 6.59-6.90 (m, 1H), 4.48-4.75 (m, 1H), 4.31 (br s, 05H), 4.10 (br s, 05H), 3.37-3.59 (m, 1H), 3.37-3.59 (m, 1H), 3.34-3.57 (m, 1H), 3.03 (br s, 05H), 2.59 (br s, 05H), 2.21-2.49 (m, 4H), 2.01-2.20 (m, 1H), 1.69-1.84 (m, 1H), 1.08-1.51 (m, 4H), 0.76-0.98 (m, 3H). LCMS [M+H]⁺=545.2 (calcd. 545.2).

### Example 82 and 83

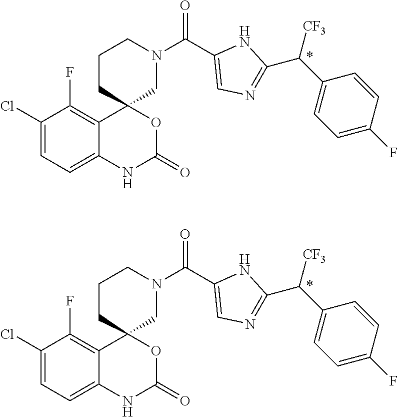 [SMILES: O=C1Nc2ccc(Cl)c(F)c2[C@@]2(CCCN(C(=O)c3cnc(C(c4ccc(F)cc4)C(F)(F)F)[nH]3)C2)O1 ; O=C1Nc2ccc(Cl)c(F)c2[C@@]2(CCCN(C(=O)c3cnc(C(c4ccc(F)cc4)C(F)(F)F)[nH]3)C2)O1]

### (R)-6-chloro-5-fluoro-1′-(2-((R)-2,2,2-trifluoro-1-(4-fluorophenyl)ethyl)-1H-imidazole-5-carbonyl)spiro[benzo[d][1,3]oxazine-4,3′-piperidin]-2(1H)-one and (R)-6-chloro-5-fluoro-1′-(2-((S)-2,2,2-trifluoro-1-(4-fluorophenyl)ethyl)-1H-imidazole-5-carbonyl)spiro[benzo[d][1,3]oxazine-4,3′-piperidin]-2(1H)-one

Step 1: (3′R)-6-Chloro-5-fluoro-1′-(2-(2,2,2-trifluoro-1-(4-fluorophenyl)-1-hydroxyethyl)-1-((2-(trimethylsilyl)ethoxy)methyl)-1H-imidazole-5-carbonyl)spiro[benzo[d][1,3]oxazine-4,3′-piperidin]-2(1H)-one. To a solution of (R)-6-chloro-5-fluoro-1′-(2-(4-fluorobenzoyl)-1-((2-(trimethylsilyl)ethoxy)methyl)-1H-imidazole-5-carbonyl)spiro[benzo[d][1,3]oxazine-4,3′-piperidin]-2(1H)-one (700 mg, 1.13 mmol) in THF (10 mL) was added trimethyl(trifluoromethyl)silane (194 mg, 1.36 mmol) and CsF (258 mg, 1.70 mmol) at rt, and the resulting mixture was stirred at rt for 1 h. The reaction was quenched with water and extracted with EtOAc. The organic layers were concentrated to afford a crude residue that was purified by silica gel chromatography (EtOAc/petroleum ether) to give the title compound. LCMS [M-58]⁺=629.2 (calcd. 687.2).

Step 2: (3′R)-6-Chloro-1′-(2-(1-chloro-2,2,2-trifluoro-1-(4-fluorophenyl)ethyl)-1H-imidazole-5-carbonyl)-5-fluorospiro[benzo[d][1,3]oxazine-4,3′-piperidin]-2(1H)-one. To a solution of (3′R)-6-chloro-5-fluoro-1′-(2-(2,2,2-trifluoro-1-(4-fluorophenyl)-1-hydroxyethyl)-1-((2-(trimethylsilyl)ethoxy)methyl)-1H-imidazole-5-carbonyl)spiro[benzo[d][1,3]oxazine-4,3′-piperidin]-2(1H-one (180 mg, 0.262 mmol) in DCM (5 mL) was added SOCl₂ (0.50 mL, 6.9 mmol) and the mixture was stirred at rt for 12 h. The reaction was concentrated to afford a crude product that was carried on without further purification. LCMS [M+H]⁺=575.1 (calcd. 575.1).

Step 3: (3′R)-6-Chloro-5-fluoro-1′-(2-(2,2,2-trifluoro-1-(4-fluorophenyl)ethyl)-1H-imidazole-5-carbonyl)spiro[benzo[d][1,3]oxazine-4,3′-piperidin]-2(1H)-one. To a solution of (3′R)-6-chloro-1′-(2-(1-chloro-2,2,2-trifluoro-1-(4-fluorophenyl)ethyl)-1H-imidazole-5-carbonyl)-5-fluorospiro[benzo[d][1,3]oxazine-4,3′-piperidin]-2(1H)-one (15 mg, 0.026 mmol) in THF (1 mL) was added LiAlH₄ (1.5 mg, 0.039 mmol) at 0° C. The resulting mixture was warmed to rt and allowed to stir for 12 h. The reaction was concentrated to afford a crude residue that was purified by reverse phase HPLC (ACN/water with 0.05% TFA modifier) to give the title compounds as a stereochemical mixture. LCMS [M+H]⁺=541.1, (calcd. 541.1). The title compounds were resolved by SFC (Column: DAICEL CHIRALPAK AD (250 mm*30 mm, 10 um); Mobile Phase A: CO₂, Mobile Phase B: EtOH (0.1% NH₃H₂O); Flow rate: 60 mL/min; Gradient 50% B to 50% B). The faster eluting isomer was obtained (Example 82): LCMS [M+H]⁺=541.1, (calcd. 541.1). ¹H NMR (400 MHz, CD₃OD) δ 7.34-7.78 (m, 4H), 6.86-7.21 (m, 2H), 6.51-6.83 (m, 1H), 6.11 (br d, J=13.2 Hz, 0.5H), 5.26-5.54 (m, 0.5H), 4.99 (br s, 1H), 4.76 (br d, J=10.3 Hz, 1H), 3.72 (br d, J=14.2 Hz, 0.5H), 2.93 (br s, 0.5H), 2.45-2.60 (m, 1H), 1.87-2.40 (m, 3H), 1.74 (br d, J=12.2 Hz, 1H). The slower eluting isomer of the title compound was obtained (Example 83): LCMS [M+H]⁺=541.1, (calcd. 541.1). ¹H NMR (400 MHz, CD₃OD) δ 7.36-7.76 (m, 4H), 6.94-7.25 (m, 2H), 6.74 (br s, 1H), 5.71 (br d, J=2.9 Hz, 0.5H), 5.12 (br s, 2H), 4.74 (br s, 0.5H), 3.76 (br s, 0.5H), 3.33-3.44 (m, 1H), 2.97 (br d, J=16.9 Hz, 0.5H), 2.54 (br s, 1H), 2.16-2.37 (m, 2H), 1.75 (br d, J=14.4 Hz, 1H).

### Example 84 and 85

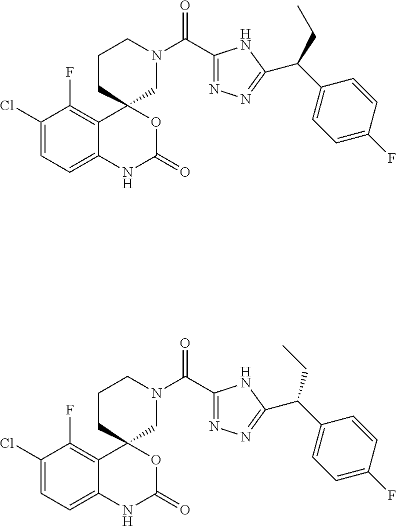 [SMILES: CC[C@@H](c1ccc(F)cc1)c1nnc(C(=O)N2CCC[C@@]3(C2)OC(=O)Nc2ccc(Cl)c(F)c23)[nH]1 ; CC[C@H](c1ccc(F)cc1)c1nnc(C(=O)N2CCC[C@@]3(C2)OC(=O)Nc2ccc(Cl)c(F)c23)[nH]1]

### (R)-6-Chloro-5-fluoro-1′-(5-((S)-1-(4-fluorophenyl)propyl)-4H-1,2,4-triazole-3-carbonyl)spiro[benzo[d][1,3]oxazine-4,3′-piperidin]-2(1H)-one and (R)-6-Chloro-5-fluoro-1′-(5-((R)-1-(4-fluorophenyl)propyl)-4H-1,2,4-triazole-3-carbonyl)spiro[benzo[d][1,3]oxazine-4,3′-piperidin]-2(1H)-one

Step 1: (3′R)-6-Chloro-5-fluoro-1′-(5-(1-(4-fluorophenyl)propyl)-4H-1,2,4-triazole-3-carbonyl)spiro[benzo[d][1,3]oxazine-4,3′-piperidin]-2(1H)-one. The title compounds were prepared as a mixture following procedures similar to those described in Example 1. LCMS [M+H]⁺=502.4 (calcd. 502.1). The title compounds were resolved by SFC (Column: DAICEL CHIRALCEL OJ-H (250 mm*30 mm, 5 um), (Condition Mobile Phase A: CO₂, Mobile Phase B: 0.1% NH₃H₂O EtOH, Begin B 20%, End B 20% Gradient Time min), 100% B Hold Time (min) FlowRate (L/min)). The faster eluting isomer of the title compound was obtained (Example 84): ¹H NMR (500 MHz, CD₃OD) δ 7.41-7.49 (m, 1H), 7.37 (dd, J=8.6, 5.4 Hz, 1H), 7.30 (br s, 1H), 7.05 (br s, 1H), 6.94 (br s, 1H), 6.64-6.79 (m, 1H), 4.90 (br s, 2H), 3.95-4.14 (m, 1H), 3.77 (br d, J=14.8 Hz, 1H), 3.33-3.46 (m, 1H), 2.99 (br t, J=12.7 Hz, 1H), 2.47-2.60 (m, 1H), 2.10-2.36 (m, 3H), 1.85-2.09 (m, 1H), 1.78 (br d, J=14.3 Hz, 1H), 1.19-1.44 (m, 2H), 0.78-0.97 (m, 3H). LCMS [M+H]⁺=502.2 (calcd. 502.1). The slower eluting isomer of the title compound was obtained (Example 85): ¹H NMR (500 MHz, CD₃OD) δ 7.45 (br t, J=7.8 Hz, 1H), 7.37 (dd, J=8.5, 5.5 Hz, 1H), 7.29 (br d, J=5.3 Hz, 1H), 7.06 (br d, J=7.8 Hz, 1H), 6.92 (br s, 1H), 6.75 (br dd, J=18.0, 8.5 Hz, 1H), 4.91-5.05 (m, 1H), 4.74-4.86 (m, 1H), 3.96-4.12 (m, 1H), 3.39-3.79 (m, 1H), 2.99 (br t, J=11.9 Hz, 1H), 2.53 (br d, J=13.7 Hz, 1H), 2.11-2.35 (m, 3H), 1.93-2.10 (m, 1H), 1.68-1.82 (m, 1H), 0.79-0.95 (m, 3H). LCMS [M+H]⁺=502.2 (calcd. 502.1).

Following procedures similar to those described above for Examples 84 and 85 and using appropriate starting materials, the following compounds were prepared.

| | | | Exact Mass | Chiral |
|---|---|---|---|---|
| Example | Structure | Name | [M + H]⁺ | Column |
| 86 | 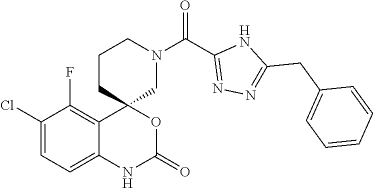 [SMILES: O=C1Nc2ccc(Cl)c(F)c2[C@@]2(CCCN(C(=O)c3nnc(Cc4ccccc4)[nH]3)C2)O1] | (R)-1′-(5-Benzyl-4H- 1,2,4-triazole-3- carbonyl)-6-chloro- 5- fluorospiro[benzo[d] [1,3]oxazine-4,3′- piperidin]-2(1H)-one | Clacd.: 455.9 Found: 456.1 | |
| 87 | 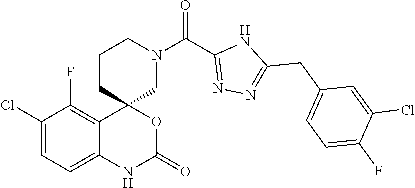 [SMILES: O=C1Nc2ccc(Cl)c(F)c2[C@@]2(CCCN(C(=O)c3nnc(Cc4ccc(F)c(Cl)c4)[nH]3)C2)O1] | (R)-6-Chloro-1′-(5- (3-chloro-4- fluorobenzyl)-4H- 1,2,4-triazole-3- carbonyl)-5- fluorospiro[benzo[d] [1,3]oxazine-4,3′- piperidin]-2(1H)-one | Calcd.: 508.1 Found: 508.1 | |
| 88 | 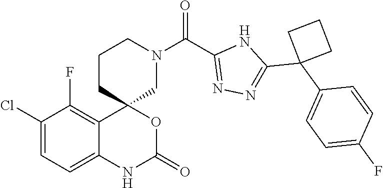 [SMILES: O=C1Nc2ccc(Cl)c(F)c2[C@@]2(CCCN(C(=O)c3nnc(C4(c5ccc(F)cc5)CCC4)[nH]3)C2)O1] | (R)-6-Chloro-5- fluoro-1′-(5-(1-(4- fluorophenyl)cyclo- butyl)-4H-1,2,4- triazole-3- carbonyl)spiro[benzo [d][1,3]oxazine-4,3′- piperidin]-2(1H)-one | Calcd.: 513.9 Found: 514.0 | |
| 89 | 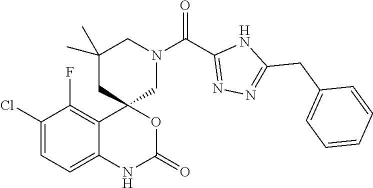 [SMILES: CC1(C)CN(C(=O)c2nnc(Cc3ccccc3)[nH]2)C[C@]2(C1)OC(=O)Nc1ccc(Cl)c(F)c12] | (R)-1′-(5-Benzyl-4H- 1,2,4-triazole-3- carbonyl)-6-chloro- 5-fluoro-5′,5′- dimethylspiro[benzo [d][1,3]oxazine-4,3′- piperidin]-2(1H)-one | Calcd.: 483.9 Found: 484.1 | |
| 90 | 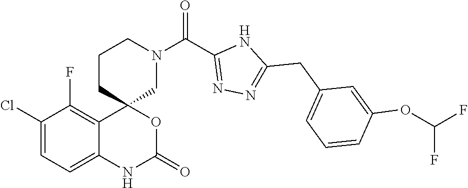 [SMILES: O=C1Nc2ccc(Cl)c(F)c2[C@@]2(CCCN(C(=O)c3nnc(Cc4cccc(OC(F)F)c4)[nH]3)C2)O1] | (R)-6-Chloro-1′-(5- (3- (difluoromethoxy) benzyl)-4H-1,2,4- triazole-3-carbonyl)- 5- fluorospiro[benzo[d] [1,3]oxazine-4,3′- piperidin]-2(1H)-one | Calcd.: 522.9 Found: 522.9 | |
| 91 | 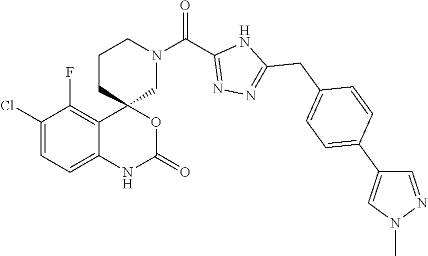 [SMILES: Cn1cc(-c2ccc(Cc3nnc(C(=O)N4CCC[C@@]5(C4)OC(=O)Nc4ccc(Cl)c(F)c45)[nH]3)cc2)cn1] | (R)-6-Chloro-5- fluoro-1′-(5-(4-(1- methyl-1H-pyrazol- 4-yl)benzyl)-4H- 1,2,4-triazole-3- carbonyl)spiro[benzo [d][1,3]oxazine-4,3′- piperidin]-2(1H)-one | Calcd.: 536.0 Found: 536.0 | |
| 92 | 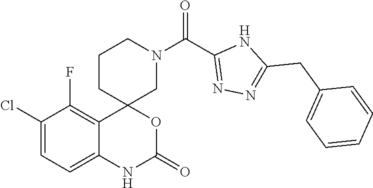 [SMILES: O=C1Nc2ccc(Cl)c(F)c2C2(CCCN(C(=O)c3nnc(Cc4ccccc4)[nH]3)C2)O1] | 1-(5-Benzyl-4H- 1,2,4-triazole-3- carbonyl)-6′-chloro- 5′-fluoro-1′H- spiro[piperidine-3,4′- quinolin]-2′(3′H)-one | Calcd.: 452.9 Found: 453.1 | |
| 93 | 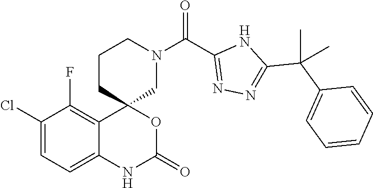 [SMILES: CC(C)(c1ccccc1)c1nnc(C(=O)N2CCC[C@@]3(C2)OC(=O)Nc2ccc(Cl)c(F)c23)[nH]1] | (R)-6-Chloro-5- fluoro-1′-(5-(2- phenylpropan-2-yl)- 4H-1,2,4-triazole-3- carbonyl)spiro[benzo [d][1,3]oxazine-4,3′- piperidin]-2(1H)-one | Calcd.: 483.9 Found: 484.1 | |
| 94 | 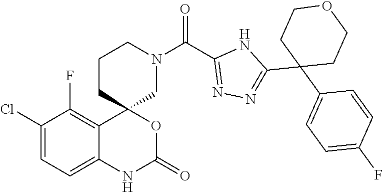 [SMILES: O=C1Nc2ccc(Cl)c(F)c2[C@@]2(CCCN(C(=O)c3nnc(C4(c5ccc(F)cc5)CCOCC4)[nH]3)C2)O1] | (R)-6-Chloro-5- fluoro-1′-(5-(4-(4- fluorophenyl)tetra- hydro-2H-pyran-4-yl)- 4H-1,2,4-triazole-3- carbonyl)spiro[benzo [d][1,3]oxazine-4,3′- piperidin]-2(1H)-one | Calcd.: 544.0 Found: 544.0 | |
| 95 | 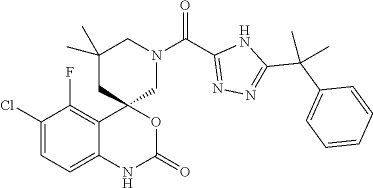 [SMILES: CC1(C)CN(C(=O)c2nnc(C(C)(C)c3ccccc3)[nH]2)C[C@]2(C1)OC(=O)Nc1ccc(Cl)c(F)c12] | (R)-6-Chloro-5- fluoro-5′,5′-dimethyl- 1′-(5-(2- phenylpropan-2-yl)- 4H-1,2,4-triazole-3- carbonyl)spiro[benzo [d][1,3]oxazine-4,3′- piperidin]-2(1H)-one | Calcd.: 512.2 Found: 512.4 | |
| 96 | 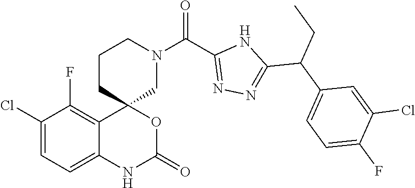 [SMILES: CCC(c1ccc(F)c(Cl)c1)c1nnc(C(=O)N2CCC[C@@]3(C2)OC(=O)Nc2ccc(Cl)c(F)c23)[nH]1] | (4R)-6-Chloro-1′-(5- (1-(3-chloro-4- fluorophenyl)propyl)- 4H-1,2,4-triazole-3- carbonyl)-5- fluorospiro[benzo[d] [1,3]oxazine-4,3′- piperidin]-2(1H)-one | Calcd.: 536.4 Found: 537.0 | OJ-H |
| 97 | 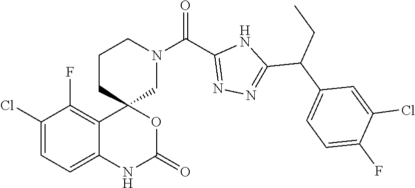 [SMILES: CCC(c1ccc(F)c(Cl)c1)c1nnc(C(=O)N2CCC[C@@]3(C2)OC(=O)Nc2ccc(Cl)c(F)c23)[nH]1] | (4R)-6-Chloro-1′-(5- (1-(3-chloro-4- fluorophenyl)propyl)- 4H-1,2,4-triazole-3- carbonyl)-5- fluorospiro[benzo[d] [1,3]oxazine-4,3′- piperidin]-2(1H)-one | Calcd.: 536.4 Found: 537.0 | OJ-H |
| 98 | 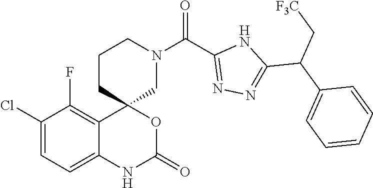 [SMILES: O=C1Nc2ccc(Cl)c(F)c2[C@@]2(CCCN(C(=O)c3nnc(C(CC(F)(F)F)c4ccccc4)[nH]3)C2)O1] | (4R)-6-Chloro-5- fluoro-1′-(5-(3,3,3- trifluoro-1- phenylpropyl)-4H- 1,2,4-triazole-3- carbonyl)spiro[benzo [d][1,3]oxazine-4,3′- piperidin]-2(1H)-one | Calcd.: 537.9 Found: 538.1 | OJ-H |
| 99 | 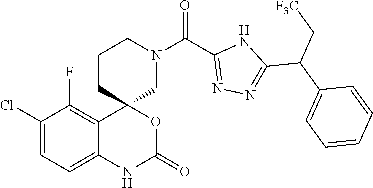 [SMILES: O=C1Nc2ccc(Cl)c(F)c2[C@@]2(CCCN(C(=O)c3nnc(C(CC(F)(F)F)c4ccccc4)[nH]3)C2)O1] | (4R)-6-Chloro-5- fluoro-1′-(5-(3,3,3- trifluoro-1- phenylpropyl)-4H- 1,2,4-triazole-3- carbonyl)spiro[benzo [d][1,3]oxazine-4,3′- piperidin]-2(1H)-one | Calcd.: 537.9 Found: 538.0 | OJ-H |
| 100 | 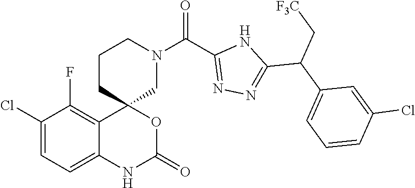 [SMILES: O=C1Nc2ccc(Cl)c(F)c2[C@@]2(CCCN(C(=O)c3nnc(C(CC(F)(F)F)c4cccc(Cl)c4)[nH]3)C2)O1] | (4R)-6-Chloro-1′-(5- (1-(3-chlorophenyl)- 3,3,3- trifluoropropyl)-4H- 1,2,4-triazole-3- carbonyl)-5- fluorospiro[benzo[d] [1,3]oxazine-4,3′- piperidin]-2(1H)-one | Calcd.: 572.4 Found: 572.1 | OJ-H |
| 101 | 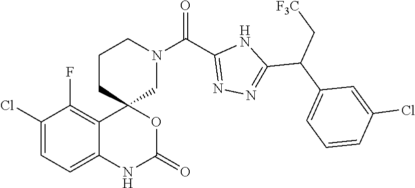 [SMILES: O=C1Nc2ccc(Cl)c(F)c2[C@@]2(CCCN(C(=O)c3nnc(C(CC(F)(F)F)c4cccc(Cl)c4)[nH]3)C2)O1] | (4R)-6-Chloro-1′-(5- (1-(3-chlorophenyl)- 3,3,3- trifluoropropyl)-4H- 1,2,4-triazole-3- carbonyl)-5- fluorospiro[benzo[d] [1,3]oxazine-4,3′- piperidin]-2(1H)-one | Calcd.: 572.4 Found: 572.0 | OJ-H |
| 102 | 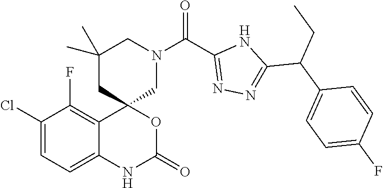 [SMILES: CCC(c1ccc(F)cc1)c1nnc(C(=O)N2CC(C)(C)C[C@@]3(C2)OC(=O)Nc2ccc(Cl)c(F)c23)[nH]1] | (4R)-6-Chloro-5- fluoro-1′-(5-(1-(4- fluorophenyl)propyl)- 4H-1,2,4-triazole-3- carbonyl)-5′,5′- dimethylspiro[benzo [d][1,3]oxazine-4,3′- piperidin]-2(1H)-one | Calcd.: 530.0 Found: 530.1 | OJ-H |
| 103 | 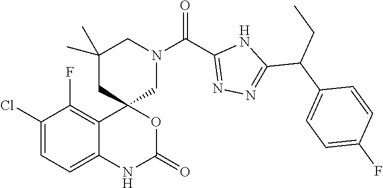 [SMILES: CCC(c1ccc(F)cc1)c1nnc(C(=O)N2CC(C)(C)C[C@@]3(C2)OC(=O)Nc2ccc(Cl)c(F)c23)[nH]1] | (4R)-6-Chloro-5- fluoro-1′-(5-(1-(4- fluorophenyl)propyl)- 4H-1,2,4-triazole-3- carbonyl)-5′,5′- dimethylspiro[benzo [d][1,3]oxazine-4,3′- piperidin]-2(1H)-one | Calcd.: 530.0 Found: 530.1 | OJ-H |
| 104 | 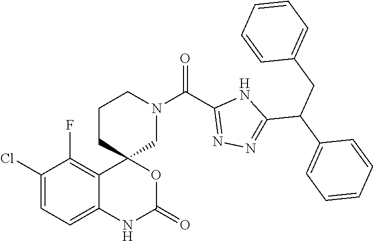 [SMILES: O=C1Nc2ccc(Cl)c(F)c2[C@@]2(CCCN(C(=O)c3nnc(C(Cc4ccccc4)c4ccccc4)[nH]3)C2)O1] | (4R)-6-Chloro-1′-(5- (1,2-diphenylethyl)- 4H-1,2,4-triazole-3- carbonyl)-5- fluorospiro[benzo[d] [1,3]oxazine-4,3′- piperidin]-2(1H)-one | Calcd.: 546.0 Found: 546.0 | LUX- CEL-4 |
| 105 | 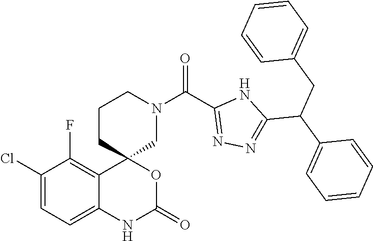 [SMILES: O=C1Nc2ccc(Cl)c(F)c2[C@@]2(CCCN(C(=O)c3nnc(C(Cc4ccccc4)c4ccccc4)[nH]3)C2)O1] | (4R)-6-Chloro-1′-(5- (1,2-diphenylethyl)- 4H-1,2,4-triazole-3- carbonyl)-5- fluorospiro[benzo[d] [1,3]oxazine-4,3′- piperidin]-2(1H)-one | Calcd.: 546.0 Found: 546.0 | LUX- CEL-4 |
| 106 | 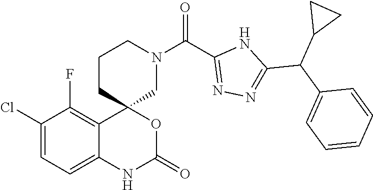 [SMILES: O=C1Nc2ccc(Cl)c(F)c2[C@@]2(CCCN(C(=O)c3nnc(C(c4ccccc4)C4CC4)[nH]3)C2)O1] | (4R)-6-Chloro-1′-(5- (cyclopropyl(phenyl) methyl)-4H-1,2,4- triazole-3-carbonyl)- 5- fluorospiro[benzo[d] [1,3]oxazine-4,3′- piperidin]-2(1H)-one | Calcd.: 495.9 Found: 496.0 | LUX- CEL-4 |
| 107 | 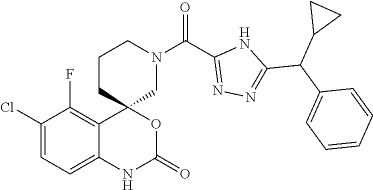 [SMILES: O=C1Nc2ccc(Cl)c(F)c2[C@@]2(CCCN(C(=O)c3nnc(C(c4ccccc4)C4CC4)[nH]3)C2)O1] | (4R)-6-Chloro-1′-(5- (cyclopropyl(phenyl) methyl)-4H-1,2,4- triazole-3-carbonyl)- 5- fluorospiro[benzo[d] [1,3]oxazine-4,3′- piperidin]-2(1H)-one | Calcd.: 495.9 Found: 496.0 | LUX- CEL-4 |
| 108 | 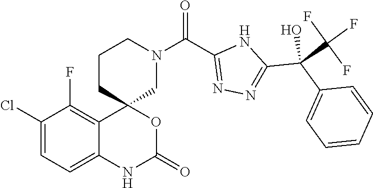 [SMILES: O=C1Nc2ccc(Cl)c(F)c2[C@@]2(CCCN(C(=O)c3nnc([C@](O)(c4ccccc4)C(F)(F)F)[nH]3)C2)O1] | (R)-6-Chloro-5- fluoro-1′-(5-((R)- 2,2,2-trifluoro-1- hydroxy-1- phenylethyl)-4H- 1,2,4-triazole-3- carbonyl)spiro[benzo [d][1,3]oxazine-4,3′- piperidin]-2(1H)-one | Calcd.: 540.1 Found: 540.1 | OJ-H |

### Example 109 and 110

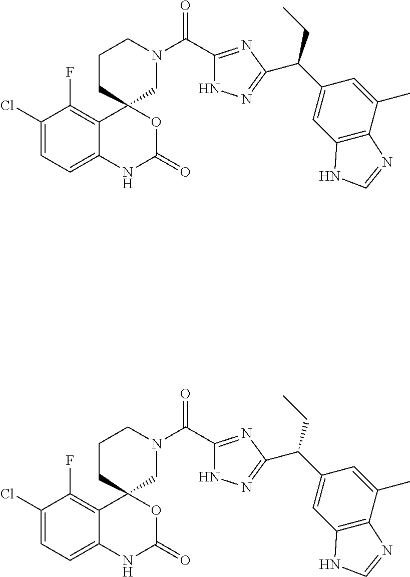 [SMILES: CC[C@@H](c1cc(C)c2nc[nH]c2c1)c1n[nH]c(C(=O)N2CCC[C@@]3(C2)OC(=O)Nc2ccc(Cl)c(F)c23)n1 ; CC[C@H](c1cc(C)c2nc[nH]c2c1)c1n[nH]c(C(=O)N2CCC[C@@]3(C2)OC(=O)Nc2ccc(Cl)c(F)c23)n1]

### (R)-6-Chloro-5-fluoro-1′-(3-((S)-1-(4-methyl-1H-benzo[d]imidazol-6-yl)propyl)-TH-1,2,4-triazole-5-carbonyl)spiro[benzo[d][1,3]oxazine-4,3′-piperidin]-2(1H)-one and (R)-6-Chloro-5-fluoro-1′-(3-((R)-1-(4-methyl-1H-benzo[d]imidazol-6-yl)propyl)-1H-1,2,4-triazole-5-carbonyl)spiro[benzo[d][1.3]oxazine-4,3′-piperidin]-2(1H)-one

Step 1: (R)-6-Chloro-1′-(3-((S)-1-chloropropyl)-1-((2-(trimethylsilyl)ethoxy)methyl)-1H-1,2,4-triazole-5-carbonyl)-5-fluorospiro[benzo[d][1,3]oxazine-4,3′-piperidin]-2(1H)-one. 1-Propanephosphonic anhydride (680 μl, 2.30 mmol) was added to a stirred solution of Intermediate M9-a (468 mg, 1.437 mmol), Intermediate A6-d (419 mg, 1.37 mmol) and TEA (600 μL, 4.31 mmol) in DCM (9 mL) at rt. After 1 h, the reaction mixture was diluted with DCM, quenched with water, and washed with satd. aq. NH₄Cl. The separated organics were dried (Na₂SO₄) and concentrated to afford a crude residue that was purified by silica gel chromatography (EtOAc/hexanes) to give the title compound. LCMS [M+Na]⁺=594.1 (calcd. 594.1).

Step 2: (4R)-6-Chloro-5-fluoro-1′-(3-(1-(4-methyl-1H-benzo[d]imidazol-6-yl)propyl)-1-((2-(trimethylsilyl)ethoxy)methyl)-1H-1,2,4-triazole-5-carbonyl)spiro[benzo[d][1,3]oxazine-4,3′-piperidin]-2(1H)-one. In glove box, 6-bromo-4-methyl-1H-benzo[d]imidazole hydrochloride (65 mg, 0.26 mmol) and 4,4′-di-tert-butyl-2,2′-bipyridine (12 mg, 0.044 mmol), nickel(II) bromide ethylene glycol dimethyl ether complex (14 mg. 0.044 mmol) were added to a stirred solution of (4R)-6-chloro-1′-(5-(1-chloropropyl)-4-((2-(trimethylsilyl)ethoxy)methyl)-4H-1,2,4-triazole-3-carbonyl)-5-fluorospiro[benzo[d][1,3]oxazine-4,3′-piperidin]-2(1H)-one (50 mg, 0.087 mmol) in DMI (0.9 mL) at rt. Zinc (17 mg, 0.26 mmol) was added, and the resulting mixture was heated to 50° C. for 1 h. A second portion of nickel(II) bromide ethylene glycol dimethyl ether complex (13.5 mg, 0.044 mmol) and zinc (17 mg, 0.26 mmol) were added, and the reaction continued stirring at 50° C. for 1 h. The reaction was cooled to rt, diluted with water, and extracted with EtOAc. The combined organic layers were washed with brine, dried (MgSO₄), and concentrated to afford a crude residue that was purified by silica gel chromatography (EtOAc/hexanes) to give the title compound. LCMS [M+H]⁺=668.1 (calcd. 668.3).

Step 3: (4R)-6-Chloro-5-fluoro-1′-(3-(1-(4-methyl-1H-benzo[d]imidazol-6-yl)propyl)-1H-1,2,4-triazole-5-carbonyl)spiro[benzo[d][1,3]oxazine-4,3′-piperidin]-2(1H)-one. HCl (60 μl, 0.24 mmol, 4 M dioxane solution) was added to a stirred solution of (4R)-6-chloro-5-fluoro-1′-(5-(1-(4-methyl-1H-benzo[d]imidazol-6-yl)propyl)-4-((2-(trimethylsilyl)ethoxy)methyl)-4H-1,2,4-triazole-3-carbonyl)spiro[benzo[d][1,3]oxazine-4,3′-piperidin]-2(1H)-one (20 mg, 0.030 mmol) in dioxane (0.5 ml) at rt. After 3 h, the reaction was concentrated, and the resulting crude residue was purified by preparative reverse phase HPLC (ACN/water+0.05% TFA) to give the title compound as a stereochemical mixture. LCMS [M+H]⁺=538.4 (calcd. 538.0).

The title compounds were resolved by SFC (Column DAICEL CHIRALCEL AS-H (250 mm*21 mm), (Condition Mobile Phase A: CO₂, Mobile Phase B: 0.2% DIPA EtOH, Begin B 35%, End 35%)). The faster eluting isomer of the title compound was obtained (Example 109): LCMS [M+H]⁺=537.9 (calcd. 538.0). ¹H NMR (400 MHz, CD₃OD) δ 8.09 (d, J=9.0 Hz, 1H). 7.50-7.29 (m, 1H), 7.04 (d, J=35.9 Hz, 1H), 6.70 (dd, J=41.0, 8.6 Hz, 1H), 5.56 (d, J=12.4 Hz, 1H), 4.17-4.01 (m, 1H), 3.79 (d, J=14 Hz, 0.5H, rotomer 1), 3.40 (d, J=14 Hz, 0.5H, rotomer 2), 3.08-2.90 (m, 1H), 2.49 (m, 3H), 2.39-1.97 (m, 2H), 1.81-1.64 (m, 1H), 1.63-1.46 (m, 1H), 1.28-1.23 (m, 3H), 1.16 (d, J=6.2 Hz, 1H), 0.89 (m, 3H).

The slower eluting isomer of the title compound was obtained (Example 110): LCMS [M+H]⁺=537.9 (calcd. 538.0). ¹H NMR (400 MHz, CD₃OD) δ 8.09 (d, J=9.0 Hz, 1H), 7.50-7.29 (m, 1H), 7.04 (d, J=35.9 Hz, 1H), 6.70 (dd, J=41.0, 8.6 Hz, 1H), 5.56 (d, J=12.4 Hz, 1H), 4.17-4.01 (m, 1H), 3.79 (d, J=14 Hz, 0.5H, rotomer 1), 3.40 (d, J=14 Hz, 0.5H, rotomer 2), 3.08-2.90 (m, 1H), 2.49 (m, 3H), 2.39-1.97 (m, 2H), 1.81-1.64 (m, 1H), 1.63-1.46 (m, 1H), 1.28-1.23 (m, 3H), 1.16 (d, J=6.2 Hz, 1H), 0.89 (m, 3H).

Following procedures similar to those described above for Examples 109 and 110 and using appropriate starting materials, the following compounds were prepared.

| | | | Exact | |
|---|---|---|---|---|
| | | | Mass | Chiral |
| Example | Structure | Name | [M + H]⁺ | Column |
| 111 | 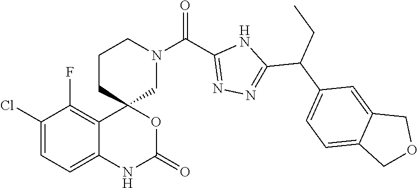 [SMILES: CCC(c1ccc2c(c1)COC2)c1nnc(C(=O)N2CCC[C@@]3(C2)OC(=O)Nc2ccc(Cl)c(F)c23)[nH]1] | (4R)-6-chloro-1′-(5-(1-(1,3- dihydroisobenzofuran-5- yl)propyl)-4H-1,2,4-triazole-3- carbonyl)-5- fluorospiro[benzo[d][1,3]oxazine- 4,3′-piperidin]-2(1H)-one | Calcd.: 526.0 Found: 526.0 | AS-H |
| 112 | 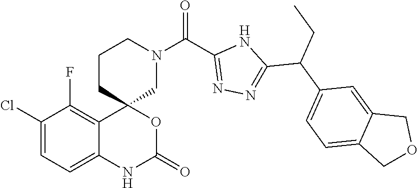 [SMILES: CCC(c1ccc2c(c1)COC2)c1nnc(C(=O)N2CCC[C@@]3(C2)OC(=O)Nc2ccc(Cl)c(F)c23)[nH]1] | (4R)-6-chloro-1′-(5-(1-(1,3- dihydroisobenzofuran-5- yl)propyl)-4H-1,2,4-triazole-3- carbonyl)-5- fluorospiro[benzo[d][1,3]oxazine- 4,3′-piperidin]-2(1H)-one | Calcd.: 526.0 Found: 526.0 | AS-H |

### Example 113

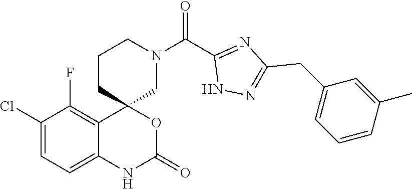 [SMILES: Cc1cccc(Cc2n[nH]c(C(=O)N3CCC[C@@]4(C3)OC(=O)Nc3ccc(Cl)c(F)c34)n2)c1]

### (R)-6-Chloro-1′-(3-(3-methylbenzyl)-1H-1,2,4-triazole-5-carbonyl)-5-fluorospiro[benzo[d][1,3]oxazine-4,3′-piperidin]-2(1H)-one

Step 1: (R)-6-Chloro-5-fluoro-1′-(5-(hydroxymethyl)-4-((2-(trimethylsilyl)ethoxy)methyl)-4H-1,2,4-triazole-3-carbonyl)spiro[benzo[d][1,3]oxazine-4,3′-piperidin]-2(1H)-one. The title compound was prepared, in two steps, from ethyl 5-(hydroxymethyl)-4-((2-(trimethylsilyl)ethoxy)methyl)-4H-1,2,4-triazole-3-carboxylate (prepared as described above in Intermediate M9-a, Steps 1-3), which was treated under conditions similar to those described above for Intermediate C5-b, Step 4, followed by treatment of the crude product therefrom with Intermediate A6-d under conditions similar to those described in Examples 1 and 2. LCMS [M+H]⁺=526.1 (calcd. 526.0).

Step 2: (R,E)-N-((5-(6-chloro-5-fluoro-2-oxo-1,2-dihydrospiro[benzo[d][1,3]oxazine-4,3′-piperidine]-1′-carbonyl)-4-((2-(trimethylsilyl)ethoxy)methyl)-4H-1,2,4-triazol-3-yl)methylene)-4-methoxybenzene sulfonohydrazide. DMP (6.15 g, 14.5 mmol) was added to a stirred mixture of (R)-6-chloro-5-fluoro-1′-(5-(hydroxymethyl)-4-((2-(trimethylsilyl)ethoxy)methyl)-4H-1,2,4-triazole-3-carbonyl)spiro[benzo[d][1,3]oxazine-4,3′-piperidin]-2(1H)-one (6.93 g, 13.2 mmol), and NaHCO₃ (1.11 g, 13.2 mmol) in DCM (66 ml) at rt. After 12 h, the reaction was partitioned between sat. aq. NaHCO₃ and EtOAc, and the resulting mixture was filtered through Celite®.

The layers were separated, and the organic layer was dried (Na₂SO₄), filtered and concentrated to afford a crude residue that was dissolved in MeOH (30 mL). To this solution, was added 4-methoxybenzenesulfonohydrazide (1.33 g, 6.57 mmol), and the resulting mixture was allowed to stir at rt. After 12 h, the reaction was concentrated to dryness, and the resulting crude residue was dissolved in EtOAc. The organics were washed with satd. aq. NH₄Cl, water, and brine, followed by drying (Na₂SO₄), filtration and concentration to afford a crude residue that was purified by silica gel chromatography (EtOAc/hexanes) to give the title compound. LCMS [M+Na]⁺=730.1 (calcd. 730.2).

Step 3: (R)-6-Chloro-1′-(3-(3-methylbenzyl)-1H-1,2,4-triazole-5-carbonyl)-5-fluorospiro[benzo[d][1,3]oxazine-4,3′-piperidin]-2(1H)-one. A mixture of (R,E)-N-((5-(6-chloro-5-fluoro-2-oxo-1,2-dihydrospiro[benzo[d][1,3]oxazine-4,3′-piperidine]-1′-carbonyl)-4-((2-(trimethylsilyl)ethoxy)methyl)-4H-1,2,4-triazol-3-yl)methylene)-4-methoxybenzene sulfonohydrazide (71 mg, 0.10 mmol), (3-methyl)boronic acid (20 mg, 0.15 mmol) and potassium carbonate (35 mg, 0.25 mmol) was suspended in dioxane (0.5 mL), and the resulting mixture was heated to 110° C. After 12 h, the reaction was cooled to rt, and an excess of HCl (4 M dioxane solution) was added. The mixture was heated to 60° C. for 1 h, cooled to rt and concentrated to afford a crude residue that was purified by and purified by reverse phase HPLC (ACN/water with 0.05% TFA modifier) to give the title compound. ¹H NMR (500 MHz, DMSO-d₆) δ 10.67 (s, 0.5H), 10.57 (s, 0.5H). 7.55 (m, 1H), 7.21-6.98, (m, 3H), 6.78 (m, 1H), 4.79 (d, J=15 Hz, 1H), 4.57 (d, J=15 Hz, 1H), 4.07 (m, 1H), 3.97 (M, 1H), 3.77 (d, J=15 Hz, 1H), 2.93 (m, 2H), 2.28 (s, 3H). 1.97-1.85 (m, 2H) 1.69 (m, 1H). LCMS [M+H]⁺=470.0 (calcd. 469.9).

Following procedures similar to those described above for Example 113 and using appropriate starting materials, the following compounds were prepared.

| | | | Exact |
|---|---|---|---|
| | | | Mass |
| Example | Structure | Name | [M + H]⁺ |
| 114 | 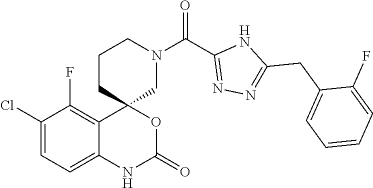 [SMILES: O=C1Nc2ccc(Cl)c(F)c2[C@@]2(CCCN(C(=O)c3nnc(Cc4ccccc4F)[nH]3)C2)O1] | (R)-6-Chloro-5-fluoro-1′-(5-(2- fluorobenzyl)-4H-1,2,4-triazole-3- carbonyl)spiro[benzo[d][1,3]oxazine- 4,3′-piperidin]-2(1H)-one | Calcd.: 473.9 Found: 474.0 |
| 115 | 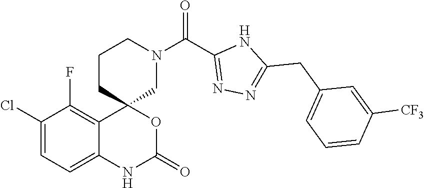 [SMILES: O=C1Nc2ccc(Cl)c(F)c2[C@@]2(CCCN(C(=O)c3nnc(Cc4cccc(C(F)(F)F)c4)[nH]3)C2)O1] | (R)-6-Chloro-5-fluoro-1′-(5-(3- (trifluoromethyl)benzyl)-4H-1,2,4- triazole-3- carbonyl)spiro[benzo[d][1,3]oxazine- 4,3′-piperidin]-2(1H)-one | Calcd.: 523.9 Found: 524.0 |
| 116 | 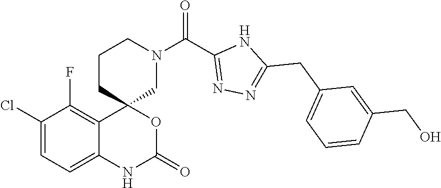 [SMILES: O=C1Nc2ccc(Cl)c(F)c2[C@@]2(CCCN(C(=O)c3nnc(Cc4cccc(CO)c4)[nH]3)C2)O1] | (R)-6-Chloro-5-fluoro-1′-(5-(3- (hydroxymethyl)benzyl)-4H-1,2,4- triazole-3- carbonyl)spiro[benzo[d][1,3]oxazine- 4,3′-piperidin]-2(1H)-one | Calcd.: 485.9 Found: 486.0 |
| 117 | 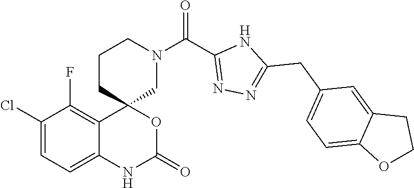 [SMILES: O=C1Nc2ccc(Cl)c(F)c2[C@@]2(CCCN(C(=O)c3nnc(Cc4ccc5c(c4)CCO5)[nH]3)C2)O1] | (R)-6-Chloro-1′-(5-((2,3- dihydrobenzofuran-5-yl)methyl)-4H- 1,2,4-triazole-3-carbonyl)-5- fluorospiro[benzo[d][1,3]oxazine- 4,3′-piperidin]-2(1H)-one | Calcd.: 497.9 Found: 498.0 |
| 118 | 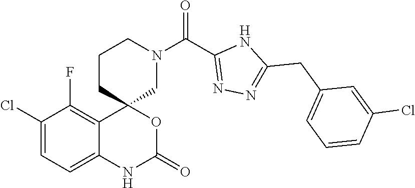 [SMILES: O=C1Nc2ccc(Cl)c(F)c2[C@@]2(CCCN(C(=O)c3nnc(Cc4cccc(Cl)c4)[nH]3)C2)O1] | (R)-6-Chloro-1′-(5-(3-chlorobenzyl)- 4H-1,2,4-triazole-3-carbonyl)-5- fluorospiro[benzo[d][1,3]oxazine- 4,3′-piperidin]-2(1H)-one | Calcd.: 490.3 Found: 490.0 |
| 119 | 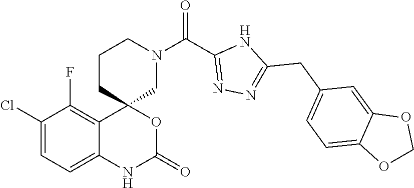 [SMILES: O=C1Nc2ccc(Cl)c(F)c2[C@@]2(CCCN(C(=O)c3nnc(Cc4ccc5c(c4)OCO5)[nH]3)C2)O1] | (R)-1′-(5-(Benzo[d][1,3]dioxol-5- ylmethyl)-4H-1,2,4-triazole-3- carbonyl)-6-chloro-5- fluorospiro[benzo[d][1,3]oxazine- 4,3′-piperidin]-2(1H)-one | Calcd.: 500.0 Found: 500.1 |
| 120 | 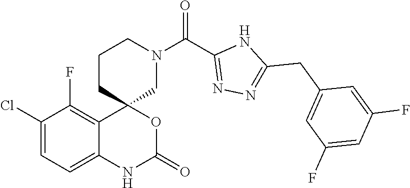 [SMILES: O=C1Nc2ccc(Cl)c(F)c2[C@@]2(CCCN(C(=O)c3nnc(Cc4cc(F)cc(F)c4)[nH]3)C2)O1] | (R)-6-Chloro-1′-(5-(3,5- difluorobenzyl)-4H-1,2,4-triazole-3- carbonyl)-5- fluorospiro[benzo[d][1,3]oxazine- 4,3′-piperidin]-2(1H)-one | Calcd.: 491.9 Found: 492.0 |
| 121 | 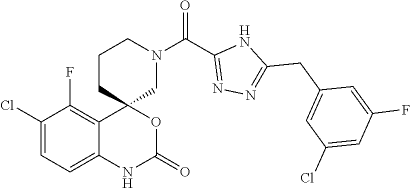 [SMILES: O=C1Nc2ccc(Cl)c(F)c2[C@@]2(CCCN(C(=O)c3nnc(Cc4cc(F)cc(Cl)c4)[nH]3)C2)O1] | (R)-6-Chloro-1′-(5-(3-chloro-5- fluorobenzyl)-4H-1,2,4-triazole-3- carbonyl)-5- fluorospiro[benzo[d][1,3]oxazine- 4,3′-piperidin]-2(1H)-one | Calcd.: 508.3 Found: 508.1 |
| 122 | 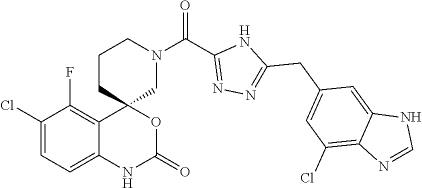 [SMILES: O=C1Nc2ccc(Cl)c(F)c2[C@@]2(CCCN(C(=O)c3nnc(Cc4cc(Cl)c5nc[nH]c5c4)[nH]3)C2)O1] | (R)-6-Chloro-1′-(5-((4-chloro-1H- benzo[d]imidazol-6-yl)methyl)-4H- 1,2,4-triazole-3-carbonyl)-5- fluorospiro[benzo[d][1,3]oxazine- 4,3′-piperidin]-2(1H)-one | Calcd.: 530.3 Found: 530.0 |
| 123 | 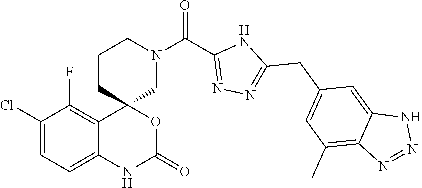 [SMILES: Cc1cc(Cc2nnc(C(=O)N3CCC[C@@]4(C3)OC(=O)Nc3ccc(Cl)c(F)c34)[nH]2)cc2[nH]nnc12] | (R)-6-Chloro-5-fluoro-1′-(5-((4- methyl-1H-benzo[d][1,2,3]triazol-6- yl)methyl)-4H-1,2,4-triazole-3- carbonyl)spiro[benzo[d][1,3]oxazine- 4,3′-piperidin]-2(1H)-one | Calcd.: 510.9 Found: 511.1 |
| 124 | 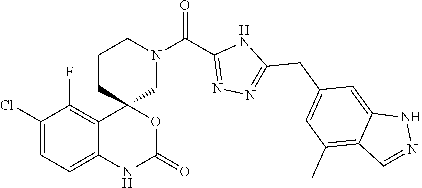 [SMILES: Cc1cc(Cc2nnc(C(=O)N3CCC[C@@]4(C3)OC(=O)Nc3ccc(Cl)c(F)c34)[nH]2)cc2[nH]ncc12] | (R)-6-Chloro-5-fluoro-1′-(5-((4- methyl-1H-indazol-6-yl)methyl)-4H- 1,2,4-triazole-3- carbonyl)spiro[benzo[d][1,3]oxazine- 4,3′-piperidin]-2(1H)-one | Calcd.: 509.9 Found: 510.0 |
| 125 | 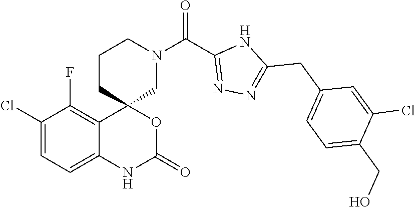 [SMILES: O=C1Nc2ccc(Cl)c(F)c2[C@@]2(CCCN(C(=O)c3nnc(Cc4ccc(CO)c(Cl)c4)[nH]3)C2)O1] | (R)-6-Chloro-1′-(5-(3-chloro-4- (hydroxymethyl)benzyl)-4H-1,2,4- triazole-3-carbonyl)-5- fluorospiro[benzo[d][1,3]oxazine- 4,3′-piperidin]-2(1H)-one | Calcd.: 520.3 Found: 520.0 |
| 126 | 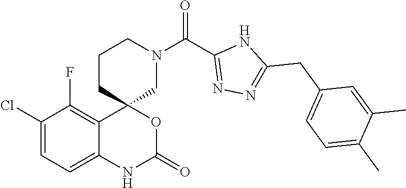 [SMILES: Cc1ccc(Cc2nnc(C(=O)N3CCC[C@@]4(C3)OC(=O)Nc3ccc(Cl)c(F)c34)[nH]2)cc1C] | (R)-6-Chloro-1′-(5-(3,4- dimethylbenzyl)-4H-1,2,4-triazole-3- carbonyl)-5- fluorospiro[benzo[d][1,3]oxazine- 4,3′-piperidin]-2(1H)-one | Calcd.: 483.9 Found: 484.0 |

### Example 127

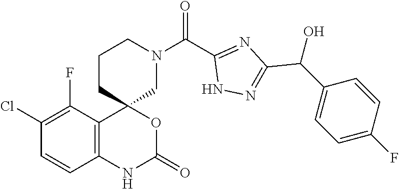 [SMILES: O=C1Nc2ccc(Cl)c(F)c2[C@@]2(CCCN(C(=O)c3nc(C(O)c4ccc(F)cc4)n[nH]3)C2)O1]

### (4R)-6-Chloro-5-fluoro-1′-(3-((4-fluorophenyl)(hydroxy)methyl)-1H-1,2,4-triazole-5-carbonyl)spiro[benzo[d][1,3]oxazine-4,3′-piperidin]-2(1H)-one

Step 1: (R)-1′-(3-Bromo-1-((2-(trimethylsilyl)ethoxy)methyl)-1H-1,2,4-triazole-5-carbonyl)-6-chloro-5-fluorospiro[benzo[d][1,3]oxazine-4,3′-piperidin]-2(1H)-one. EDC (1.64 g, 8.57 mmol) was added to a stirred solution of Intermediate M-a (2.34 g, 7.14 mmol) and Intermediate A6-d (2.63 g, 7.14 mmol) in pyridine (25 mL), and the resulting mixture was allowed to stir at rt. After 12 h, the reaction was concentrated, and the resulting crude residue was purified by silica gel chromatography (silica, EtOAc:petroleum ether) to give the title compound. LCMS [M-57]*=516.0 (calcd. M+H=574.1).

Step 2: (R)-5-(6-chloro-5-fluoro-2-oxo-1,2-dihydrospiro[benzo[d][1,3]oxazine-4,3′-piperidin]-1′-ylcarbonyl)-1-((2-(trimethylsilyl)ethoxy)methyl)-1H-1,2,4-triazole-3-carbaldehyde. The title compound was prepared in two steps following procedures similar to those described for Intermediate H, Steps 4 and 5. LCMS [M+H]⁺=524.2 (calcd. 524.2).

Step 3: (3′R)-6-Chloro-5-fluoro-1′-(3-((4-fluorophenyl)(hydroxy)methyl)-1-((2-(trimethylsilyl)ethoxy)methyl)-1H-1,2,4-triazole-5-carbonyl)spiro[benzo[d][1,3]oxazine-4,3′-piperidin]-2(1H)-one. n-Butyllithium (820 μL, 2.06 mmol, 2.5 M hexanes solution) was added dropwise to a stirred solution of 1-bromo-4-fluorobenzene (300 mg, 1.71 mmol) in THF (10 mL) at −78° C. After 30 min, (R)-5-(6-chloro-5-fluoro-2-oxo-1,2-dihydrospiro[benzo[d][1,3]oxazine-4,3′-piperidin]-1′-ylcarbonyl)-1-((2-(trimethylsilyl)ethoxy)methyl)-1H-1,2,4-triazole-3-carbaldehyde (800 mg, 1.527 mmol) was added, and the resulting mixture was allowed to stir at −78° C. for 1 h. The reaction was quenched by addition of satd. aq. NH₄Cl and extracted with EtOAc. The combined organic layers were concentrated to afford a crude residue that was purified by preparative TLC (EtOAc/petroleum ether) to give the title compound. LCMS [M+H]⁺=620.2 (calcd. 620.2).

Step 4: (3′R)-6-Chloro-5-fluoro-1′-(3-((4-fluorophenyl)(hydroxy)methyl)-1H-1,2,4-triazole-5-carbonyl)spiro[benzo[d][1,3]oxazine-4,3′-piperidin]-2(1H)-one. The title compound was prepared following procedures similar to those described above for Examples 75, Step 5. ¹H NMR (400 MHz, CD₃OD) δ 7.50 (dd, J=8.1, 5.6 Hz, 1H), 7.39-7.45 (m, 2H), 7.08 (br t, J=8.6 Hz, 1H), 6.97 (td, J=8.8, 2.4 Hz, 1H), 6.66-6.79 (m, 1H), 5.96 (s, 0.5H), 5.90 (s, 0.5H), 5.41-5.58 (m, 0.5H), 4.97 (br d, J=13.9 Hz, 1H), 4.75 (br d, J=12.7 Hz, 0.5H), 3.79 (dd, J=14.2, 4.9 Hz, 0.5H), 3.40 (br d, J=14.2 Hz, 0.5H), 2.91-3.03 (m, 1H), 2.38-2.57 (m, 1H), 2.13-2.35 (m, 2H), 1.63-1.84 (m, 1H), 1.62-1.82 (m, 1H). LCMS [M+H]⁺=490.2 (calcd. 490.1).

### Examples 128

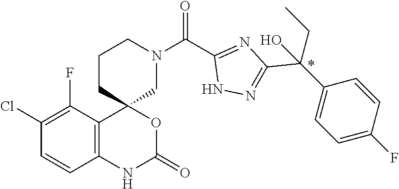 [SMILES: CCC(O)(c1ccc(F)cc1)c1n[nH]c(C(=O)N2CCC[C@@]3(C2)OC(=O)Nc2ccc(Cl)c(F)c23)n1]

### (R)-6-Chloro-5-fluoro-1′-(3-(UR or S)-1-(4-fluorophenyl)-1-hydroxypropyl)-1H-1,2,4-triazole-5-carbonyl)spiro[benzo[d][1,3]oxazine-4,3′-piperidin]-2(1H)-one

Step 1: (R)-6-Chloro-5-fluoro-1′-(3-(4-fluorobenzoyl)-1H-1,2,4-triazole-5-carbonyl)spiro[benzo[d][1,3]oxazine-4,3′-piperidin]-2(1H)-one. The title compound was prepared following procedures similar to those described above in Examples 1 and 2. LCMS [M+H]⁺=488.1 (calcd. 488.1).

Step 2: (3′R)-6-Chloro-5-fluoro-1′-(3-(1-(4-fluorophenyl)-1-hydroxypropyl)-1H-1,2,4-triazole-5-carbonyl)spiro[benzo[d][1,3]oxazine-4,3′-piperidin]-2(1H)-one. Ethylmagnesium bromide (0.31 mL, 0.92 mmol, 3 M THF solution) was added to a stirred solution of (R)-6-Chloro-5-fluoro-1′-(3-(4-fluorobenzoyl)-1H-1,2,4-triazole-5-carbonyl)spiro[benzo[d][1,3]oxazine-4,3′-piperidin]-2(1H)-one (233 mg, 0.450 mmol) in THF (6 mL) at rt. After 3 h, the reaction was diluted with water and extracted with a 10% MeOH:DCM mixture. The combined organics were concentrated, and the resulting crude residue was purified by and purified by reverse phase HPLC (ACN/water with 0.05% TFA modifier) to give the title compound as a stereochemical mixture. LCMS [M+H]⁺=518.0 (calcd. 518.1).

The title compounds were resolved by SFC (Column: DAICEL CHIRALPAK AD (250 mm*30 mm, 10 um); Mobile Phase A: CO₂, Mobile Phase B: EtOH (0.1% NH₃H₂O); Flow rate: 60 mL/min; Gradient 50% B to 50% B). The faster eluting isomer of the title compound was obtained (Example 128): ¹H NMR (400 MHz, CD₃OD) δ 7.50-7.67 (m, 2H), 7.44 (q, J=8.2 Hz, 1H), 7.04 (t, J=8.6 Hz, 1H), 6.92 (t, J=8.8 Hz, 1H), 6.66-6.79 (m, 1H), 5.45 (s, 0.5H), 5.01 (br d, J=14.1 Hz, 0.5H), 4.80 (br d, J=14.5 Hz, 1H), 3.78 (d, J=14.5 Hz, 1H), 3.41 (d, J=13.7 Hz, 0.5H), 2.93-3.05 (m, 0.5H), 2.09-2.45 (m, 4H), 2.02-2.70 (m, 1H), 1.67-1.82 (m, 1H), 0.75-0.89 (m, 3H). LCMS [M+H]⁺=518.0 (calcd. 518.1).

### Example 129

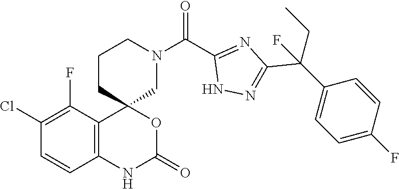 [SMILES: CCC(F)(c1ccc(F)cc1)c1n[nH]c(C(=O)N2CCC[C@@]3(C2)OC(=O)Nc2ccc(Cl)c(F)c23)n1]

### (3′R)-6-Chloro-5-fluoro-1′-(3-(1-fluoro-1-(4-fluorophenyl)propyl)-1H-1,2,4-triazole-5-carbonyl)spiro[benzo[d][1.3]oxazine-4,3′-piperidin]-2(1H)-one

Step 1: (3′R)-6-Chloro-5-fluoro-1′-(3-(1-fluoro-1-(4-fluorophenyl)propyl)-1H-1,2,4-triazole-5-carbonyl)spiro[benzo[d][1,3]oxazine-4,3′-piperidin]-2(1H)-one. DAST (23 μL, 0.17 mmol) was added to a stirred mixture of (3′R)-6-chloro-5-fluoro-1′-(3-(1-(4-fluorophenyl)-1-hydroxypropyl)-1H-1,2,4-triazole-5-carbonyl)spiro[benzo[d][1,3]oxazine-4,3′-piperidin]-2(1H)-one (30 mg, 0.058 mmol) in DCM (0.8 mL) at 0° C. The resulting mixture was warmed to rt and allowed to stir for 12 h, at which time, the reaction was diluted with water and extracted with DCM. The combined organics were concentrated, and the resulting crude residue was purified by and purified by reverse phase HPLC (ACN/water with 0.05% TFA modifier) to give the title compound.

The title compounds were resolved by SFC (Column: DAICEL CHIRALCEL AD-H (250 mm*30 mm, 5 um), Mobile Phase A: CO₂, Mobile Phase B: MeOH (0.1% NH₃H₂O); Flow rate: 60 mL/min; Gradient 20% B to 20% B). The faster eluting isomer of the title compound was obtained (Example 129): ¹H NMR (400 MHz, CD₃OD) δ 7.49-7.56 (m, 1H), 7.48-7.55 (m, 1H), 7.34-7.48 (m, 2H), 7.12 (t, J=9.0 Hz, 1H), 6.92-7.04 (m, 1H), 6.64-6.81 (m, 1H), 5.48-5.67 (m, 0.5H), 5.00 (br d, J=13.7 Hz, 1H), 4.77 (br d, J=12.9 Hz, 0.5H), 3.75 (dd, J=14.1, 4.7 Hz, 1H), 3.42 (d, J=13.7 Hz, 0.5H), 2.93-3.05 (m, 0.5H), 2.43-2.65 (m, 2H), 2.07-2.38 (m, 3H), 1.69-1.83 (m, 1H), 0.92 (t, J=7.4 Hz, 1H), 0.85 (q, J=7.0 Hz, 2H). LCMS [M+H]⁺=520.2 (calcd. 520.1).

### Example 130

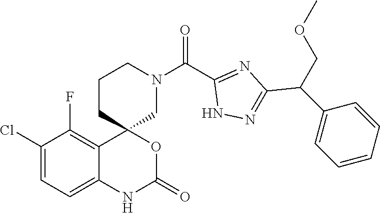 [SMILES: COCC(c1ccccc1)c1n[nH]c(C(=O)N2CCC[C@@]3(C2)OC(=O)Nc2ccc(Cl)c(F)c23)n1]

### (4R)-6-Chloro-5-fluoro-1′-(3-(2-methoxy-1-phenylethyl)-1H-1,2,4-triazole-5-carbonyl)spiro[benzo[d][1,3]oxazine-4,3′-piperidin]-2(1H)-one

Step 1: (R,Z)-6-Chloro-5-fluoro-1′-(3-(2-methoxy-1-phenylvinyl)-1H-1,2,4-triazole-5-carbonyl)spiro[benzo[d][1,3]oxazine-4,3′-piperidin]-2(1H)-one. LHMDS (0.57 mL, 0.75 mmol, 1.4 M THE solution) was added to a stirred solution of (methoxymethyl)triphenylphosphonium chloride (219 mg, 0.638 mmol) in THF (5 mL) at −78° C. After 30 min (R)-1′-(3-benzoyl-TH-1,2,4-triazole-5-carbonyl)-6-chloro-5-fluorospiro[benzo[d][1,3]oxazine-4,3′-piperidin]-2(1H)-one (250 mg, 0.532 mmol) was added, and the resulting mixture was warmed to 0° C. and allowed to stir for 2 h. The reaction was quenched with satd. aq. NH₄Cl and extracted with EtOAc. The combined organic layers were concentrated to afford a crude residue that was purified by silica gel chromatography (MeOH/DCM) to give the title compound. LCMS [M+H]⁺=498.1 (calcd. 498.1).

Step 2: 6-Chloro-5-fluoro-1′-(3-(2-methoxy-1-phenylethyl)-1H-1,2,4-triazole-5-carbonyl)spiro[benzo[d][1,3]oxazine-4,3′-piperidin]-2(1H)-one. Raney Ni (106 mg, 0.181 mmol) was added to a stirred solution of (Z)-6-chloro-5-fluoro-1′-(3-(2-methoxy-1-phenylvinyl)-1H-1,2,4-triazole-5-carbonyl)spiro[benzo[d][1,3]oxazine-4,3′-piperidin]-2(1H)-one (30 mg, 0.036 mmol) in THF (2 mL), and the resulting mixture was allowed to stir at rt for 12 h. The reaction mixture was filtered through a pad of Celite® and the filtrate was concentrated to afford a crude residue that was purified by and purified by reverse phase HPLC (ACN/water with 0.05% TFA modifier) to give the title compound. ¹H NMR (400 MHz, CD₃OD) δ 7.40-7.49 (m, 1H), 7.26-7.39 (m, 3H), 7.15-7.26 (m, 2H), 6.64-6.84 (m, 1H), 5.50-5.69 (m, 0.5H), 5.00 (br d, J=14.2 Hz, 0.5H), 4.79 (br d, J=13.0 Hz, 1H), 4.38-4.57 (m, 1H), 3.99-4.16 (m, 1H), 3.70-3.96 (m, 0.5H), 3.41-3.50 (m, 1H), 3.36 (s, 1H), 3.26 (d, J=11.7 Hz, 2H), 2.94-3.08 (m, 0.5H), 2.45-2.60 (m, 1H), 2.10-2.38 (m, 2H), 1.68-1.83 (m, 1H). LCMS [M+H]⁺=500.2 (calcd. 500.1).

### Example 131

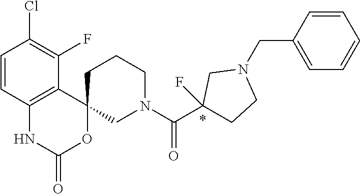 [SMILES: O=C1Nc2ccc(Cl)c(F)c2[C@@]2(CCCN(C(=O)C3(F)CCN(Cc4ccccc4)C3)C2)O1]

### (R)-1′-((S or R)-1-Benzyl-3-fluoropyrrolidine-3-carbonyl)-6-chloro-5-fluorospiro[benzo[d][1,3]oxazine-4,3′-piperidin]-2(1H)-one

Step 1: (4R)-1′-(1-Benzyl-3-fluoropyrrolidine-3-carbonyl)-6-chloro-5-fluorospiro[benzo[d][1,3]oxazine-4,3′-piperidin]-2(1H)-one. DIEA (0.171 ml, 0.977 mmol) was added to a stirred solution of Intermediate A6-d (100 mg, 0.326 mmol), potassium 1-benzyl-3-fluoropyrrolidine-3-carboxylate (100 mg, 0.383 mmol) and HATU (161 mg, 0.423 mmol) in DMF (2.5 ml), and the resulting mixture was allowed to stir at rt. After 12 h, the reaction was diluted with EtOAc, washed with water and brine. The organic layer was dried (MgSO₄), filtered and then concentrated to afford a crude residue that purified by silica gel chromatography ((25% EtOH:EtOAc)/hexanes) to give the title compound as a mixture of isomers.

The title compound was resolved by SFC (Column OJ-H 50×250 mm, Conditions: Mobile Phase A: CO₂, Mobile Phase B: 0.10% DIPA MeOH). The slower eluting isomer as the title compound was obtained (Example 131): ¹H NMR (500 MHz, DMSO-d₆) δ 10.62 (s, 1H), 7.55 (t, J=8.2 Hz, 1H), 7.37-7.22 (m, 5H), 6.79 (t, J=9.2 Hz, 1H), 4.14-4.66 (dd, 2H), 3.57-3.76 (m, 4H), 2.96-3.27 (m, 2H), 2.69-2.91 (m, 2H), 2.33-2.58 (m, 2H), 2.05-2.26 (m, 2H), 1.64-1.90 (m, 2H). LCMS [M+H]⁺=476.1 (calcd. 476.2).

### Example 132

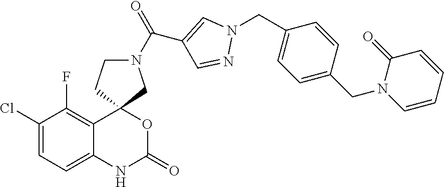 [SMILES: O=C1Nc2ccc(Cl)c(F)c2[C@]2(CCN(C(=O)c3cnn(Cc4ccc(Cn5ccccc5=O)cc4)c3)C2)O1]

### (S)-6-Chloro-5-fluoro-1′-(1-(4-(2-oxopyridin-1(2H)-yl)methyl)benzyl)-1H-pyrazole-4-carbonyl)spiro[benzo[d][1,3]oxazine-4,3′-pyrrolidin]-2(1H)-one

Step 1: (S)-6-Chloro-5-fluoro-1′-(1-(4-((2-oxopyridin-1(2H)-yl)methyl)benzyl)-1H-pyrazole-4-carbonyl)spiro[benzo[d][1,3]oxazine-4,3′-pyrrolidin]-2(1H)-one. 1-(4-((2-Oxopyridin-1(2H)-yl)methyl)benzyl)-1H-pyrazole-4-carboxylic acid (48 mg, 0.15 mmol) was added to a stirred solution of Intermediate A6-c (38 mg, 0.10 mmol) and DIEA (90 μL, 0.51 mmol) in DMF (2 mL). HATU (59 mg, 0.15 mmol) was added, and the resulting mixture was allowed to stir at rt. After 2 h, the reaction was filtered and purified by and purified by reverse phase HPLC (ACN/water with 0.05% TFA modifier) to give the title compound. LCMS [M+H]⁺=548.1 (calcd. 548.2). ¹H NMR (600 MHz, DMSO-d₆) δ 10.73 (d, J=19.7 Hz, 1H), 8.41 (d, J=35.2 Hz, 1H), 7.88 (s, 1H), 7.83-7.65 (m, 1H), 7.56 (t, J=8.2 Hz, 1H), 7.41 (s, 1H), 7.23 (dd, J=21.4, 10.2 Hz, 3H), 6.80 (t, J=8.1 Hz, 1H), 6.39 (d, J=8.2 Hz, 1H), 6.22 (d, J=6.4 Hz, 1H), 5.32 (d, J=31.3 Hz, 2H), 5.07 (d, J=9.4 Hz, 2H), 4.21 (d, J=15.0 Hz, 1H), 4.03 (t, J=12.0 Hz, 1H), 4.00-3.88 (m, 1H), 3.80 (t, J=10.1 Hz, 1H), 3.67 (d, J=7.1 Hz, 1H), 2.72-2.57 (m, 1H).

Following procedures similar to those described above for Example 132 and using appropriate starting materials, the following compounds were prepared.

| | | | Exact Mass | Chiral |
|---|---|---|---|---|
| Example | Structure | Name | [M + H]⁺ | Column |
| 133 | 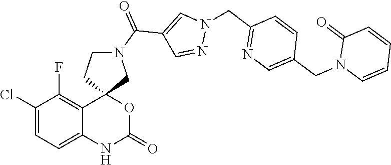 [SMILES: O=C1Nc2ccc(Cl)c(F)c2[C@]2(CCN(C(=O)c3cnn(Cc4ccc(Cn5ccccc5=O)cn4)c3)C2)O1] | (S)-6-Chloro-5-fluoro-1′-(1-((5-((2- oxopyridin-1(2H)-yl)methyl)pyridin- 2-yl)methyl)-1H-pyrazole-4- carbonyl)spiro[benzo[d][1,3]oxazine- 4,3′-pyrrolidin]-2(1H)-one | Calcd.: 549.0 Found: 549.0 | |
| 134 | 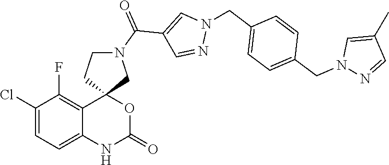 [SMILES: Cc1cnn(Cc2ccc(Cn3cc(C(=O)N4CC[C@]5(C4)OC(=O)Nc4ccc(Cl)c(F)c45)cn3)cc2)c1] | (S)-6-Chloro-5-fluoro-1′-(1-(4-((4- methyl-1H-pyrazol-1- yl)methyl)benzyl)-1H-pyrazole-4- carbonyl)spiro[benzo[d][1,3]oxazine- 4,3′-pyrrolidin]-2(1H)-one | Calcd.: 535.0 Found: 535.1 | |
| 135 | 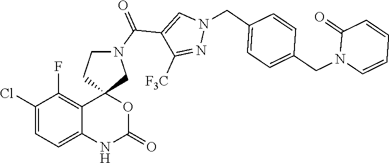 [SMILES: O=C1Nc2ccc(Cl)c(F)c2[C@]2(CCN(C(=O)c3cn(Cc4ccc(Cn5ccccc5=O)cc4)nc3C(F)(F)F)C2)O1] | (S)-6-Chloro-5-fluoro-1′-(1-(4-((2- oxopyridin-1(2H)-yl)methyl)benzyl)- 3-(trifluoromethyl)-1H-pyrazole-4- carbonyl)spiro[benzo[d][1,3]oxazine- 4,3′-pyrrolidin]-2(1H)-one | Calcd.: 616.0 Found: 616.0 | |
| 136 | 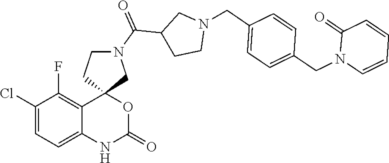 [SMILES: O=C1Nc2ccc(Cl)c(F)c2[C@]2(CCN(C(=O)C3CCN(Cc4ccc(Cn5ccccc5=O)cc4)C3)C2)O1] | (4S)-6-Chloro-5-fluoro-1′-(1-(4-((2- oxopyridin-1(2H)- yl)methyl)benzyl)pyrrolidine-3- carbonyl)spiro[benzo[d][1,3]oxazine- 4,3′-pyrrolidin]-2(1H)-one | Calcd.: 551.3 Found: 551.2 | CHIRAL ART Cellulose- SB |
| 137 | 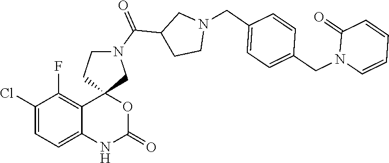 [SMILES: O=C1Nc2ccc(Cl)c(F)c2[C@]2(CCN(C(=O)C3CCN(Cc4ccc(Cn5ccccc5=O)cc4)C3)C2)O1] | (4S)-6-Chloro-5-fluoro-1′-(1-(4-((2- oxopyridin-1(2H)- yl)methyl)benzyl)pyrrolidine-3- carbonyl)spiro[benzo[d][1,3]oxazine- 4,3′-pyrrolidin]-2(1H)-one | Calcd.: 551.3 Found: 551.2 | CHIRAL ART Cellulose- SB |
| 138 | 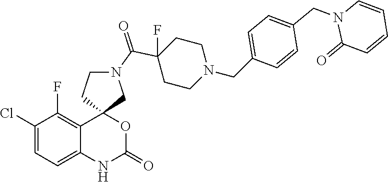 [SMILES: O=C1Nc2ccc(Cl)c(F)c2[C@]2(CCN(C(=O)C3(F)CCN(Cc4ccc(Cn5ccccc5=O)cc4)CC3)C2)O1] | (4S)-6-Chloro-5-fluoro-1′-(4-fluoro- 1-(4-((2-oxopyridin-1(2H)- yl)methyl)benzyl)piperidine-4- carbonyl)spiro[benzo[d][1,3]oxazine- 4,3′-pyrrolidin]-2(1H)-one | Calcd.: 583.0 Found: 583.1 | |

### Factor XIa Assay

The effectiveness of a compound of the present invention as an inhibitor of Coagulation factor XIa can be determined using a relevant purified serine protease, and an appropriate synthetic substrate. The rate of hydrolysis of the chromogenic or fluorogenic substrate by the relevant serine protease was measured both in the absence and presence of compounds of the present invention. Assays were conducted at rt or at 37° C. Hydrolysis of the substrate resulted in release of amino trifluoromethylcoumarin (AFC), which was monitored spectrofluorometrically by measuring the increase in emission at 510 nm with excitation at 405 nm. A decrease in the rate of fluorescence change in the presence of inhibitor is indicative of enzyme inhibition. Such methods are known to one skilled in the art. The results of this assay are expressed as the half-maximal inhibitory concentrations (IC₅₀), or the inhibitory constant, Kᵢ.

Compounds were pre-incubated for 30 min at 25° C. with human (0.04 nM) factor XIa in 50 mM HEPES buffer with 150 mM sodium chloride, 5 mM calcium chloride, 0.1% PEG 8000, pH 7.4. factor XIa enzymatic activity was determined by addition of the substrate glycine-proline-arginine-7-amido-4-trifluoromethylcoumarin (GPR-AFC) and measurement of the fluorescence at 400/505 nm after a 60 min incubation at 25° C. The % inhibition for each data point was calculated from the data and analyzed using the log (inhibitor) vs. response four parameters equation to determine the half-maximal inhibitory concentrations (IC₅₀). The IC₅₀ were converted to equilibrium inhibitory constants (Ki) using the Cheng-Prusoff equation.

The activities shown by this assay indicate that the compounds of the invention may be therapeutically useful for treating or preventing various cardiovascular and/or cerebrovascular thromboembolic conditions in patients suffering from unstable angina, acute coronary syndrome, refractory angina, myocardial infarction, transient ischemic attacks, atrial fibrillation, stroke such as thrombotic stroke or embolic stroke, venous thrombosis, coronary and cerebral arterial thrombosis, cerebral and pulmonary embolism, atherosclerosis, deep vein thrombosis, disseminated intravascular coagulation, and reocclusion or restenosis of recanalized vessels.

### Plasma Kallikrein Assay

The effectiveness of a compound of the present invention as an inhibitor of plasma kallikrein can be determined using a relevant purified serine protease, and an appropriate synthetic substrate. The rate of hydrolysis of the chromogenic or fluorogenic substrate by the relevant serine protease was measured both in the absence and presence of compounds of the present invention. Assays were conducted at rt or at 37° C. Hydrolysis of the substrate resulted in release of amino trifluoromethylcoumarin (AFC), which was monitored spectrofluorometrically by measuring the increase in emission at 510 nm with excitation at 405 nm. A decrease in the rate of fluorescence change in the presence of inhibitor is indicative of enzyme inhibition. Such methods are known to one skilled in the art. The results of this assay are expressed as the half-maximal inhibitory concentrations (IC₅₀), or the inhibitory constant, Kᵢ.

Plasma kallikrein determinations were made in 50 mM HEPES buffer at pH 7.4 containing 150 mM NaCl, 5 mM CaCl₂), and 0.1% PEG 8000 (polyethylene glycol; Fisher Scientific). Determinations were made using purified Human plasma kallikrein at a final concentration of 0.5 nM (Enzyme Research Laboratories) and the synthetic substrate, Acetyl-K-P-R-AFC (Sigma #C6608) at a concentration of 100 mM.

Activity assays were performed by diluting a stock solution of substrate at least tenfold to a final concentration ≤0.2 Km into a solution containing enzyme or enzyme equilibrated with inhibitor. Times required to achieve equilibration between enzyme and inhibitor were determined in control experiments. The reactions were performed under linear progress curve conditions and fluorescence increase measured at 405 Ex/510 Em nm. Values were converted to percent inhibition of the control reaction (after subtracting 100% Inhibition value). IC₅₀ was determined by inflection point from a four parameter logistic curve fit. Ki was calculated using the Cheng Prusoff equation, Ki=IC₅₀/(1+([S]/Km)).

The activities shown by this assay indicate that the compounds of the invention may be therapeutically useful for treating or preventing various ophthalmic, cardiovascular and/or cerebrovascular thromboembolic conditions in patients suffering from unstable angina, acute coronary syndrome, refractory angina, myocardial infarction, transient ischemic attacks, atrial fibrillation, stroke such as thrombotic stroke or embolic stroke, venous thrombosis, coronary and cerebral arterial thrombosis, cerebral and pulmonary embolism, atherosclerosis, deep vein thrombosis, disseminated intravascular coagulation, reocclusion or restenosis of recanalized vessels, hereditary angioedema, uveitis, posterior uveitis, wet age-related macular degeneration, diabetic macular edema, diabetic retinopathy and retinal vein occlusion.

Plasma Kallikrein (PKal) IC₅₀ (nM) and Factor XIa IC₅₀ (nM) for selected compounds are as follows:

| Example | PKal IC₅₀ (nM) | Factor XIa IC₅₀ (nM) |
|---|---|---|
| 1 | 6.9 | 1567 |
| 2 | 3.2 | 206 |
| 3 | 37.0 | 1375 |
| 4 | 20.0 | 10000 |
| 5 | 23.1 | 10000 |
| 6 | 8.0 | 10000 |
| 7 | 37.5 | 881 |
| 8 | 34.1 | 10000 |
| 9 | 29.7 | 5590 |
| 10 | 26.8 | 10000 |
| 11 | 20.0 | 4334 |
| 12 | 26.4 | 10000 |
| 13 | 37.6 | 10000 |
| 14 | 16.2 | 1533 |
| 15 | 38.1 | 2707 |
| 16 | 40.3 | 1453 |
| 17 | 37.8 | 10000 |
| 18 | 50.4 | 10000 |
| 19 | 27.0 | 10000 |
| 20 | 28.1 | 10000 |
| 21 | 12.1 | 10000 |
| 22 | 7.9 | 1374 |
| 23 | 46.0 | 10000 |
| 24 | 38.3 | 10000 |
| 25 | 10.4 | 210 |
| 26 | 6.9 | 7640 |
| 27 | 10.9 | 10000 |
| 28 | 10.4 | 10000 |
| 29 | 6.9 | 1567 |
| 30 | 5.7 | 4350 |
| 31 | 9.0 | 2455 |
| 32 | 4.6 | 1793 |
| 33 | 4.2 | 4763 |
| 34 | 13.1 | 270 |
| 35 | 2.6 | 1015 |
| 36 | 2.8 | 2487 |
| 37 | 2.6 | 1454 |
| 38 | 1.8 | 2056 |
| 39 | 4.0 | 1348 |
| 40 | 7.1 | 1002 |
| 41 | 33.8 | 10000 |
| 42 | 14.1 | 5235 |
| 43 | 27.1 | 4673 |
| 44 | 5.9 | 2422 |
| 45 | 0.8 | 1417 |
| 46 | 1.2 | 846 |
| 47 | 27.1 | 10000 |
| 48 | 32.6 | 10000 |
| 49 | 47.0 | 10000 |
| 50 | 13.7 | 10000 |
| 51 | 35.1 | 10000 |
| 52 | 24.1 | 10000 |
| 53 | 18.5 | 10000 |
| 54 | 29.8 | 10000 |
| 55 | 17.1 | 10000 |
| 56 | 10.9 | 10000 |
| 57 | 29.7 | 10000 |
| 58 | 10.7 | 10000 |
| 59 | 22.9 | 10000 |
| 60 | 14.7 | 10000 |
| 61 | 36.5 | 10000 |
| 62 | 49.5 | 10000 |
| 63 | 42.6 | 10000 |
| 64 | 24.2 | 10000 |
| 65 | 19.3 | 1770 |
| 66 | 32.5 | 1359 |
| 67 | 38.2 | 6269 |
| 68 | 12.4 | 6584 |
| 69 | 13.2 | 6435 |
| 70 | 5.7 | 946 |
| 71 | 5.5 | 995 |
| 72 | 9.3 | 1433 |
| 73 | 8.2 | 2048 |
| 74 | 10.8 | 2755 |
| 75 | 5.7 | 322 |
| 76 | 16.1 | 4583 |
| 77 | 56.6 | 8126 |
| 78 | 13.7 | 3851 |
| 79 | 1.7 | 194 |
| 80 | 1.9 | 179 |
| 81 | 8.7 | 2299 |
| 82 | 42.5 | 7395 |
| 83 | 21.3 | 2701 |
| 84 | 4.5 | 10000 |
| 85 | 16.7 | 3106 |
| 86 | 24.8 | 6890 |
| 87 | 49.7 | 10000 |
| 88 | 13.7 | 4440 |
| 89 | 14.9 | 7201 |
| 90 | 33.4 | 10000 |
| 91 | 25.2 | 10000 |
| 92 | 43.1 | 8778 |
| 93 | 29.9 | 3159 |
| 94 | 5.6 | 2489 |
| 95 | 21.4 | 3015 |
| 96 | 3.5 | 5084 |
| 97 | 13.8 | 8745 |
| 98 | 1.3 | 362 |
| 99 | 9.7 | 1369 |
| 100 | 2.2 | 1453 |
| 101 | 10.4 | 4008 |
| 102 | 3.6 | 1131 |
| 103 | 7.0 | 1953 |
| 104 | 16.6 | 2991 |
| 105 | 17.3 | 3599 |
| 106 | 1.6 | 795 |
| 107 | 11.2 | 1908 |
| 108 | 20.1 | 10000 |
| 109 | 1.3 | 646 |
| 110 | 1.0 | 1219 |
| 111 | 3.8 | 2822 |
| 112 | 1.0 | 1562 |
| 113 | 18.3 | 10000 |
| 114 | 33.1 | 10000 |
| 115 | 23.9 | 10000 |
| 116 | 39.9 | 10000 |
| 117 | 34.8 | 10000 |
| 118 | 29.2 | 10000 |
| 119 | 47.1 | 10000 |
| 120 | 21.1 | 10000 |
| 121 | 35.4 | 10000 |
| 122 | 17.5 | 10000 |
| 123 | 19.1 | 10000 |
| 124 | 13.5 | 7758 |
| 125 | 20.3 | 10000 |
| 126 | 33.1 | 10000 |
| 127 | 39.9 | 10000 |
| 128 | 4.0 | 382 |
| 129 | 8.9 | 4303 |
| 130 | 5.3 | 1364 |
| 131 | 9.4 | 10000 |
| 132 | 6.1 | 1000 |
| 133 | 15.1 | 10000 |
| 134 | 20.8 | 1000 |
| 135 | 7.5 | 1000 |
| 136 | 43.2 | 10000 |
| 137 | 31.0 | 1000 |
| 138 | 5.6 | 10000 |

1. A compound of the Formula I: 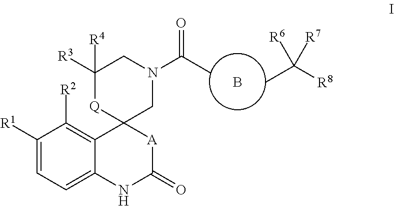
wherein A is O or —CH₂—; [symbol]
is 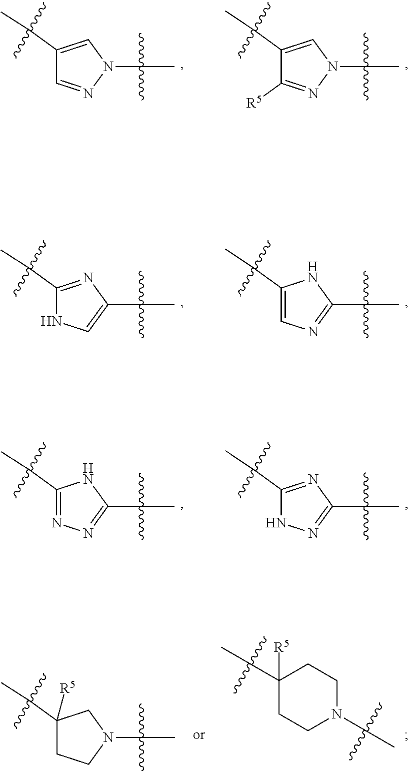
Q is —CH₂— or absent;
R¹ is selected from the group consisting of hydrogen, halo, hydroxy and C₁₋₆ alkyl;
R² is selected from the group consisting of hydrogen, halo, hydroxy and C₁₋₆ alkyl;
R³ is selected from the group consisting of hydrogen, halo, hydroxy, C₁₋₆ alkyl and C₃₋₆ cycloalkyl;
R⁴ is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3 to 6 membered cycloalkyl group, or a 5 to 6 membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

2. The compound of claim 1 wherein Q is —CH₂—, or a pharmaceutically acceptable salt thereof.

3. The compound of claim 1 of the Formula Ia: 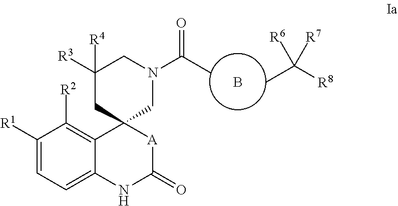
wherein A is O or —CH₂—; [symbol]
is 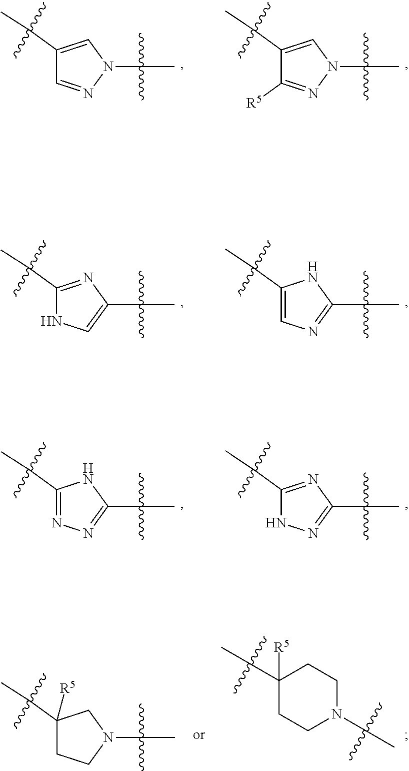
R¹ is selected from the group consisting of hydrogen, halo, hydroxy and C₁₋₆ alkyl;
R² is selected from the group consisting of hydrogen, halo, hydroxy and C₁₋₆ alkyl;
R³ is selected from the group consisting of hydrogen, halo, hydroxy, C₁₋₆ alkyl and C₃₋₆ cycloalkyl;
R⁴ is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3 to 6 membered cycloalkyl group, or a 5 to 6 membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

4. The compound of claim 1 wherein A is O, or a pharmaceutically acceptable salt thereof.

5. The compound of claim 1 of the Formula 1b: 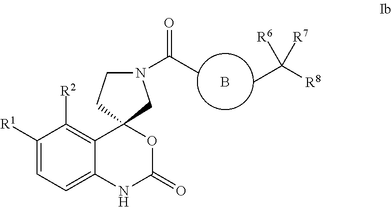
wherein [symbol] is 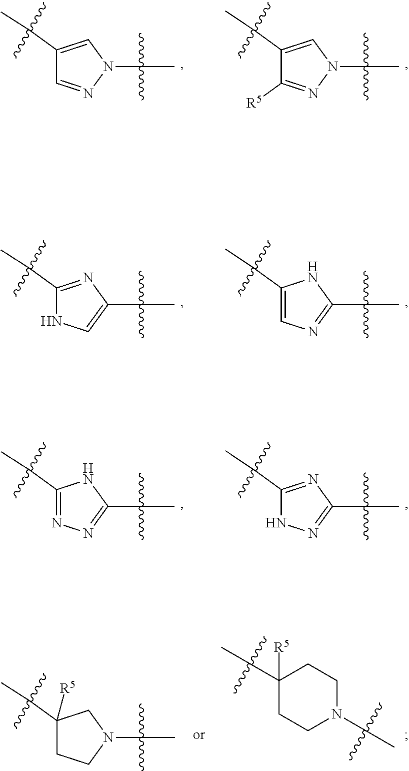
R¹ is selected from the group consisting of hydrogen, halo, hydroxy and C₁₋₆ alkyl;
R² is selected from the group consisting of hydrogen, halo, hydroxy and C₁₋₆ alkyl;
R⁵ is hydrogen, halo or C₁₋₆ alkyl, wherein said alkyl group is optionally substituted with one to three halo;
R⁶ is independently selected from the group consisting of hydrogen, halo, hydroxy, cyclopropyl, C₁₋₆ alkyl and (C₁₋₆ alkyl)cyclopropyl, wherein said alkyl group is optionally substituted with one to three substituents independently selected from the group consisting of halo, phenyl and OR^{x}, and said cyclopropyl groups are optionally substituted with OR^{x};
R⁷ is selected from the group consisting of hydrogen, halo, hydroxy and C₁₋₆ alkyl, wherein said alkyl group is optionally substituted with one to three halo or hydroxy;
or R⁶ and R⁷ can be taken together with the carbon atom to which they are attached to form a 3- to 6-membered cycloalkyl group, or a 5- to 6-membered heterocyclyl group;
R⁸ is selected from the group consisting of phenyl or heteroaryl, which can be monocyclic or bicyclic; wherein said phenyl and heteroaryl groups are optionally substituted with one to three substituents independently selected from the group consisting of oxo, halo, cyano, R^{x}, OR^{x}, NR⁹R¹⁰, (C═O)OR^{x}, OCH₂(C═O)OR^{x}, SO₂R^{x}, SO₂NR⁹R¹⁰, R^{y} and CH₂R^{y};
R⁹ is hydrogen or C₁₋₃ alkyl;
R¹⁰ is hydrogen or C₁₋₃ alkyl;
R^{x} is hydrogen or C₁₋₆ alkyl, which is optionally substituted with one to three substituents selected from the group consisting of halo and hydroxy,
R^{y} is heteroaryl, heterocyclyl or C₃₋₆ cycloalkyl, wherein said heteroaryl group is optionally substituted with oxo or C₁₋₆ alkyl, said heterocyclyl group is optionally substituted with one or two oxo and said cycloalkyl group is optionally substituted with C₁₋₆ alkyl;
or a pharmaceutically acceptable salt thereof.

6. The compound of claim 1 wherein R¹ is halo and R² is halo, or a pharmaceutically acceptable salt thereof.

7. The compound of claim 1 wherein R³ is hydrogen or methyl, R⁴ is hydrogen or methyl, or a pharmaceutically acceptable salt thereof.

8. The compound of claim 1 wherein R⁵ is hydrogen or halo, or a pharmaceutically acceptable salt thereof.

9. The compound of claim 1 wherein R⁸ is phenyl, which is optionally substituted with oxo, halo, cyano, —OCH₂(C═O)OR^{x}, —SO₂R^{x}, and R^{y}, or a pharmaceutically acceptable salt thereof.

10. The compound of claim 1 selected from any one of compounds 1-138, or a pharmaceutically acceptable salt thereof.

11. A pharmaceutical composition comprising a compound of claim 1 or a pharmaceutically acceptable salt thereof and a pharmaceutically acceptable carrier.

12. A method for treating impaired visual activity, diabetic retinopathy, diabetic macular edema, retinal vein occlusion, hereditary angioedema, diabetes, pancreatitis, cerebral hemorrhage, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery, or bleeding from postoperative surgery in a mammal, comprising administering a composition of claim 11 to a mammal in need of thereof.

13. A method for treating uveitis, posterior uveitis, wet age-related macular degeneration, diabetic macular edema, diabetic retinopathy or retinal vein occlusion in a mammal comprising administering a composition of claim 11 to a mammal in need thereof.

14. A method of treating diabetic retinopathy or diabetic macular edema in a mammal comprising administering a composition of claim 11 to a mammal in need thereof.

15. A method of treating retinal vein occlusion in a mammal comprising administering a composition of claim 11 to a mammal in need thereof.

16. (canceled)

17. (canceled)

18. The composition of claim 11 further comprising another agent selected from the group consisting of anti-inflammatory agents, anti-VEGF agents, immunosuppressive agents, anticoagulants, antiplatelet agents, and thrombolytic agents.

19. The method of claim 12 further comprising another agent selected from the group consisting of anti-inflammatory agents, anti-VEGF agents, immunosuppressive agents, anticoagulants, antiplatelet agents, and thrombolytic agents.
